(19) United States
(12) Patent Application Publication
(10) Pub. No.: US 2022/0040349 A1
(43) Pub. Date: Feb. 10, 2022

(54) ANTIVIRAL METHODS AND COMPOSITIONS

(71) Applicant: STC.UNM, Albuquerque, NM (US)

(72) Inventors: David G. Whitten, Albuquerque, NM (US); Kirk S. Schanze, Gainesville, FL (US); Linnea K. Ista, Albuquerque, NM (US); Patrick L. Donabedian, Gainesville, FL (US); Eva Yung Hua Chi, Albuquerque, NM (US); Florencia A. Monge, Albuquerque, NM (US); Alison Meredith Kell, Albuquerque, NM (US)

(21) Appl. No.: 17/452,296

(22) Filed: Oct. 26, 2021

Related U.S. Application Data
(63) Continuation-in-part of application No. PCT/US2021/043462, filed on Jul. 28, 2021.
(60) Provisional application No. 63/057,705, filed on Jul. 28, 2020.
(60) Provisional application No. 63/086,209, filed on Oct. 1, 2020.

(51) Int. Cl. A61L 2/18; A61L 2/08; A61L 2/10; A61L 9/14; A61L 9/20

(52) U.S. Cl. CPC A61L 2/18; A61L 2/084; A61L 2/10; A61L 9/14; A61L 2101/44; A61L 2202/15; A61L 2202/24; A61L 2202/25; A61L 9/20

(57) ABSTRACT

A method of inactivating an enveloped ssRNA virus, such as SARS-CoV-2. The method involves contacting the enveloped ssRNA virus with an antiviral polyelectrolyte compound and/or a conjugated aromatic compound effective to inactivate the virus. The disclosure also provides a method of reducing the period of viability of enveloped ssRNA virus, such as SARS-CoV-2, on personal protective equipment, in air, in water, and/or on surfaces that have come into contact with the virus.

### CROSS-REFERENCE TO RELATED APPLICATIONS

This application is a continuation-in-part of PCT/US2021/043462 filed Jul. 28, 2021, which claims the benefit of priority to U.S. Provisional Patent Application Ser. No. 63/057,705 filed Jul. 28, 2020, and to U.S. Provisional Patent Application Ser. No. 63/086,209 filed Oct. 1, 2020, the disclosures of each of which are incorporated herein in their entirety by reference.

### STATEMENT OF GOVERNMENT SUPPORT

This invention was made with Government support under Grant No. 1207362 awarded by the National Science Foundation. The U.S. Government has certain rights in this invention.

### BACKGROUND

The World Health Organization (WHO) has declared the novel coronavirus outbreak of 2019 to be a global pandemic. To date, severe acute respiratory syndrome coronavirus 2 (SARS-CoV-2) has infected more than 15 million people globally, and claimed more than a half million lives. Although several vaccines have been developed that are very effective against SARS-CoV-2, a strong division has arisen between pro-vaccination activists who are enthusiastic about the several vaccines that are >90% effective, and a significant component of the world population that are strongly anti-vaccination and resistant to appeals to take the vaccine. Although a high percentage of the world population has taken the vaccine, it appears that getting a sufficient fraction to be vaccinated and thus create “herd” immunity is likely impossible for the current SARS-CoV-2 and future viral pathogens.

The virus is thought to spread via respiratory droplets and by viral contamination of surfaces. As such, government and health authorities have urged the use of personal protective equipment, such as masks, the avoidance of indoor congregation, and disinfection of surfaces.

There is a need to for agents for use in materials and methods that destroy, inactivate, and/or reduce spread of coronavirus (e.g., SARS-CoV-2), such as minimizing its spread via surface contact and air. Currently there are few treatment options and very few long-lasting disinfectants available for inactivating the virus before it can spread and infect humans. While masks and protective clothing and “social distancing” may offer some protection, their use has not always halted or slowed the spread.

### SUMMARY OF THE INVENTION

The present disclosure provides method of destroying, inactivating, reducing the virality of, or otherwise inhibiting the activity of an enveloped ssRNA virus. The enveloped ssRNA virus can be SARS-CoV-2. The method includes contacting the virus with a polyelectrolyte compound, optionally in the presence of light. For example, the method can involve contacting the virus with a compound having the structure of Formula I-Formula V. The method can further involve incubating the virus and the compound together.

The present invention provides a method of inactivating SARS-CoV-2 virus. The method includes contacting SARS-CoV-2 virus with a conjugated aromatic compound effective to inactivate the virus.

The present disclosure also provides a method of reducing SARS-CoV-2 viability on a substrate, including treating the substrate with a polyelectrolyte compound, such as a compound having the structure of Formulas I-V, optionally in the presence of light. The substrate can be a fabric, a work surface, a medical device, packaging materials, personal protective equipment, a fibrous support, a wipe, a water filter, an air filter, a mask, or a combination thereof.

The present disclosure further provides a method of reducing the ability of SARS-CoV-2 to spread via a surface, including treating a substrate surface with a polyelectrolyte compound, such as a compound having the structure of Formulas I-V, optionally in the presence of light. The substrate can be a fabric, a work surface, a medical device, packaging materials, personal protective equipment, a fibrous support, a wipe, a water filter, an air filter, a mask, or a combination thereof.

The present disclosure yet further provides a method of destroying, inactivating, reducing the virality of, or otherwise inhibiting the activity of SARS-CoV-2 in circulated air. The method can involve contacting a filter with a polyelectrolyte compound, such as a compound having the structure of Formulas I-V; and exposing the filter to SARS-CoV-2 from circulated air, optionally in the presence of light. The method can further involve incubating the virus and the compound together, for example, by heating the filter at a temperature of about 35° C. for a period of at least 60 minutes.

The compound can have the structure of Formula I or Formula II:

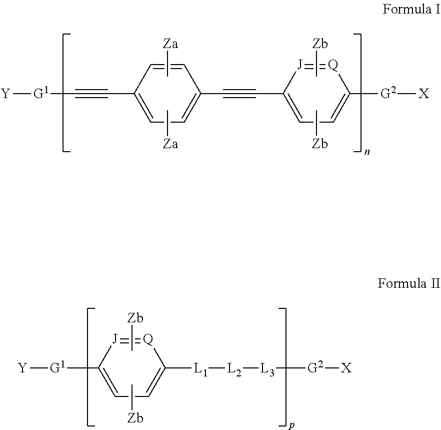

wherein

each of X and Y is independently H, COOR, O—(CH₂)ₘ-T, NH₂, or COR;

each of Za and Zb is independently H, O—(CH₂)ₘ-T, O—C₂H₄(OCH₂)ₘ—R;

each of G¹ and G² is independently a bond, C₂C₆H₄, C₄H₄, C₂C₄S, or C₄S;

J and Q are each C or CH so as to provide a benzene ring, or J and Q are together S so as to provide a thiophene ring;

n is 1 to 200;

p is 1 to 10,000;

m is 0 to 10;

each of R is independently methyl, ethyl, n-propyl, isopropyl, phenyl, t-butyl, isobutyl, n-butyl, pentyl, hexyl, heptyl, octyl, cyclopropyl, cyclobutyl, cyclopentyl, cyclyhexyl, methoxymethyl, or trifluoromethyl;

each of T is independently H, SO₃⁻, COO⁻, COOR, DABCO, N-alkyl DABCO, imidazolyl, N-alkyl imidazolyl, NR₂, NHR₂⁺, or NR₃⁺;

L1 is independently a bond or —C≡C—;

L2 is independently a bond, a substituted or unsubstituted phenylene, thiophenylene, azulenylene, heptalenylene, biphenylene, indacenylene, fluorenylene, phenanthrenylene, triphenylenylene, pyrenylene, naphthacenylene, chrysenylene, biphenylenylene, anthracenylene, and naphthylene;

L3 is independently a bond or —C≡C—; and

at least one occurrence of Y, X, Za, and Zb is independently O—(CH₂)ₘ-T.

Advantages, some of which are unexpected, are achieved by various aspects of the present disclosure. Surprisingly, in various aspects, the presently described methods and compositions can inactivate an enveloped ssRNA virus, namely, SARS-CoV-2. Moreover, various aspects of the present disclosure can provide antiviral activity visible light and/or UV light, via a mechanism that is not vulnerable to resistance arises due to viral strain mutation, or mutation in envelope surface proteins. Without intending to limit to theory, it is surprising that the agents described herein appear to demonstrate surface interactions with the SARS-CoV-2 virus, and that contemporaneous light treatment destroys, inactivates, or reduces the virality of the SARS-CoV-2 virus. For example, SARS-CoV-2 virus treated according to the presently described methods and compositions, can exhibit reduced plaque forming units, thus indicating destruction of the virus, inactivation of the virus, or reduced ability of the virus to spread. Moreover, the presently described compositions can be versatilely used to provide disinfection of SARS-CoV-2, or can be used to provide surfaces that are hostile to SARS-CoV-2 and reduce the period on which the virus can retain activity.

### BRIEF DESCRIPTION OF THE FIGURES

The drawings illustrate generally, by way of example, but not by way of limitation, various aspects of the present invention.

FIG. 1 shows a photograph of a cell monolayer infected with SARS-CoV-2 and having a number of viral plaques (light areas) amongst healthy cells (dark area), as indicated using a crystal violet solution, in accordance with various aspects.

FIGS. 2A-2D shows graph showing concentration of plaque forming units after treatment with Compound A, Compound B, Compound C, or Compound D, with FIG. 2A illustrating Trial 1, FIG. 2B illustrating Trial 2, FIG. 2C illustrating Trial 3, and with FIG. 2D illustrating a summary of the three trials, in accordance with various aspects.

FIG. 3 shows absorbance measurements for Compound A, Compound B, Compound C, or Compound D, recorded on a Lambda 35 U/Vis spectrometer from PerkinElmer (Waltham, Mass.) in quartz cuvettes, in accordance with various aspects.

FIG. 4 illustrates structures of oligomers and polymers used in the Examples, in accordance with various aspects.

FIG. 5 illustrates absorption spectra of conjugated aromatic compounds used in the Examples, in accordance with various aspects.

FIG. 6 illustrates a photograph of a crystal violet stain of a Vero E6 cell monolayer 3 days post SARS-CoV-2 infection, in accordance with various aspects.

FIGS. 7A-7D illustrate antiviral activity of conjugated aromatic compounds against SARS-CoV-2 in log pfu/mL with near UV light irradiation for various time periods, in accordance with various aspects.

FIGS. 8A-8D illustrate antiviral activity of conjugated aromatic compounds in log pfu/mL with near UV light or visible light irradiation for various times, in accordance with various aspects.

FIG. 9 illustrates an emission spectrum of an LZC-Vis lamp used for irradiation, in accordance with various aspects.

FIG. 10 illustrates antiviral activity of 10 μg/mL of Compound E in solution against SARS-CoV-2, in accordance with various aspects.

FIG. 11 illustrates antiviral activity of glass fiber filter coated with Compound E against SARS-CoV-2, in accordance with various aspects.

### DETAILED DESCRIPTION OF THE INVENTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

In this document, the terms “a,” “an,” or “the” are used to include one or more than one unless the context clearly dictates otherwise. The term “or” is used to refer to a nonexclusive “or” unless otherwise indicated. The statement “at least one of A and B” or “at least one of A or B” has the same meaning as “A, B, or A and B.” In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section.

In the methods described herein, the acts can be carried out in any order without departing from the principles of the invention, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, within 10%, within 5%, or within 1% of a stated value or of a stated limit of a range, and includes the exact stated value or range. The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The term “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

The term “organic group” as used herein refers to any carbon-containing functional group. Examples can include an oxygen-containing group such as an alkoxy group, aryloxy group, aralkyloxy group, oxo(carbonyl) group; a carboxyl group including a carboxylic acid, carboxylate, and a carboxylate ester; a sulfur-containing group such as an alkyl and aryl sulfide group; and other heteroatom-containing groups. Non-limiting examples of organic groups include OR, OOR, OC(O)N(R)₂, CN, CF₃, OCF₃, R, C(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, C(═NOR)R, and substituted or unsubstituted (C₁-C₁₀₀)hydrocarbyl, wherein R can be hydrogen (in examples that include other carbon atoms) or a carbon-based moiety, and wherein the carbon-based moiety can be substituted or unsubstituted.

The term “substituted” as used herein in conjunction with a molecule or an organic group as defined herein refers to the state in which one or more hydrogen atoms contained therein are replaced by one or more non-hydrogen atoms. The term “functional group” or “substituent” as used herein refers to a group that can be or is substituted onto a molecule or onto an organic group. Examples of substituents or functional groups include, but are not limited to, a halogen (e.g., F, Cl, Br, and I); an oxygen atom in groups such as hydroxy groups, alkoxy groups, aryloxy groups, aralkyloxy groups, oxo(carbonyl) groups, carboxyl groups including carboxylic acids, carboxylates, and carboxylate esters; a sulfur atom in groups such as thiol groups, alkyl and aryl sulfide groups, sulfoxide groups, sulfone groups, sulfonyl groups, and sulfonamide groups; a nitrogen atom in groups such as amines, hydroxyamines, nitriles, nitro groups, N-oxides, hydrazides, azides, and enamines; and other heteroatoms in various other groups. Non-limiting examples of substituents that can be bonded to a substituted carbon (or other) atom include F, Cl, Br, I, OR, OC(O)N(R)₂, CN, NO, NO₂, ONO₂, azido, CF₃, OCF₃, R, O (oxo), S (thiono), C(O), S(O), methylenedioxy, ethylenedioxy, N(R)₂, SR, SOR, SO₂R, SO₂N(R)₂, SO₃R, C(O)R, C(O)C(O)R, C(O)CH₂C(O)R, C(S)R, C(O)OR, OC(O)R, C(O)N(R)₂, OC(O)N(R)₂, C(S)N(R)₂, (CH₂)₀₋₂N(R)C(O)R, (CH₂)₀₋₂N(R)N(R)₂, N(R)N(R)C(O)R, N(R)N(R)C(O)OR, N(R)N(R)CON(R)₂, N(R)SO₂R, N(R)SO₂N(R)₂, N(R)C(O)OR, N(R)C(O)R, N(R)C(S)R, N(R)C(O)N(R)₂, N(R)C(S)N(R)₂, N(COR)COR, N(OR)R, C(═NH)N(R)₂, C(O)N(OR)R, and C(═NOR)R, wherein R can be hydrogen or a carbon-based moiety; for example, R can be hydrogen, (C₁-C₁₀₀)hydrocarbyl, alkyl, acyl, cycloalkyl, aryl, aralkyl, heterocyclyl, heteroaryl, or heteroarylalkyl; or wherein two R groups bonded to a nitrogen atom or to adjacent nitrogen atoms can together with the nitrogen atom or atoms form a heterocyclyl.

The term “alkyl” as used herein refers to straight chain and branched alkyl groups and cycloalkyl groups having from 1 to 40 carbon atoms, 1 to about 20 carbon atoms, 1 to 12 carbons, or from 1 to 8 carbon atoms. Examples of straight chain alkyl groups include those with from 1 to 8 carbon atoms such as methyl, ethyl, n-propyl, n-butyl, n-pentyl, n-hexyl, n-heptyl, and n-octyl groups. Examples of branched alkyl groups include, but are not limited to, isopropyl, iso-butyl, sec-butyl, t-butyl, neopentyl, isopentyl, and 2,2-dimethylpropyl groups. As used herein, the term “alkyl” encompasses n-alkyl, isoalkyl, and anteisoalkyl groups as well as other branched chain forms of alkyl. Representative substituted alkyl groups can be substituted one or more times with any of the groups listed herein, for example, amino, hydroxy, cyano, carboxy, nitro, thio, alkoxy, and halogen groups.

The term “alkenyl” as used herein refers to straight and branched chain and cyclic alkyl groups as defined herein, except that at least one double bond exists between two carbon atoms. Thus, alkenyl groups have from 2 to 40 carbon atoms, or 2 to about 20 carbon atoms, or 2 to 12 carbon atoms, or from 2 to 8 carbon atoms. Examples include, but are not limited to vinyl, —CH═CH(CH₃), —CH═C(CH₃)₂, —C(CH₃)═CH₂, —C(CH₃)═CH(CH₃), —C(CH₂CH₃)═CH₂, cyclohexenyl, cyclopentenyl, cyclohexadienyl, butadienyl, pentadienyl, and hexadienyl among others.

The term “alkynyl” as used herein refers to straight and branched chain alkyl groups, except that at least one triple bond exists between two carbon atoms. Thus, alkynyl groups have from 2 to 40 carbon atoms, 2 to about 20 carbon atoms, or from 2 to 12 carbons, or from 2 to 8 carbon atoms. Examples include, but are not limited to —C≡CH, —C≡C(CH₃), —C≡C(CH₂CH₃), —CH₂C≡CH, —CH₂C≡C(CH₃), and —CH₂C≡C(CH₂CH₃) among others.

The term “acyl” as used herein refers to a group containing a carbonyl moiety wherein the group is bonded via the carbonyl carbon atom. The carbonyl carbon atom is bonded to a hydrogen forming a “formyl” group or is bonded to another carbon atom, which can be part of an alkyl, aryl, aralkyl cycloalkyl, cycloalkylalkyl, heterocyclyl, heterocyclylalkyl, heteroaryl, heteroarylalkyl group or the like. An acyl group can include 0 to about 12, 0 to about 20, or 0 to about 40 additional carbon atoms bonded to the carbonyl group. An acyl group can include double or triple bonds within the meaning herein. An acryloyl group is an example of an acyl group. An acyl group can also include heteroatoms within the meaning herein. A nicotinoyl group (pyridyl-3-carbonyl) is an example of an acyl group within the meaning herein. Other examples include acetyl, benzoyl, phenylacetyl, pyridylacetyl, cinnamoyl, and acryloyl groups and the like. When the group containing the carbon atom that is bonded to the carbonyl carbon atom contains a halogen, the group is termed a “haloacyl” group. An example is a trifluoroacetyl group.

The term “aryl” as used herein refers to cyclic aromatic hydrocarbon groups that do not contain heteroatoms in the ring. Thus aryl groups include, but are not limited to, phenyl, azulenyl, heptalenyl, biphenyl, indacenyl, fluorenyl, phenanthrenyl, triphenylenyl, pyrenyl, naphthacenyl, chrysenyl, biphenylenyl, anthracenyl, and naphthyl groups. In some aspects, aryl groups contain about 6 to about 14 carbons in the ring portions of the groups. Aryl groups can be unsubstituted or substituted, as defined herein. Representative substituted aryl groups can be mono-substituted or substituted more than once, such as, but not limited to, a phenyl group substituted at any one or more of 2-, 3-, 4-, 5-, or 6-positions of the phenyl ring, or a naphthyl group substituted at any one or more of 2- to 8-positions thereof.

The term “heterocyclyl” as used herein refers to aromatic and non-aromatic ring compounds containing three or more ring members, of which one or more is a heteroatom such as, but not limited to, N, O, and S.

The term “heteroaryl” as used herein refers to aromatic ring compounds containing 5 or more ring members, of which, one or more is a heteroatom such as, but not limited to, N, O, and S; for instance, heteroaryl rings can have 5 to about 8-12 ring members. A heteroaryl group is a variety of a heterocyclyl group that possesses an aromatic electronic structure.

The term “alkoxy” as used herein refers to an oxygen atom connected to an alkyl group, including a cycloalkyl group, as are defined herein. Examples of linear alkoxy groups include but are not limited to methoxy, ethoxy, propoxy, butoxy, pentyloxy, hexyloxy, and the like. Examples of branched alkoxy include but are not limited to isopropoxy, sec-butoxy, tert-butoxy, isopentyloxy, isohexyloxy, and the like. Examples of cyclic alkoxy include but are not limited to cyclopropyloxy, cyclobutyloxy, cyclopentyloxy, cyclohexyloxy, and the like. An alkoxy group can include about 1 to about 12, about 1 to about 20, or about 1 to about 40 carbon atoms bonded to the oxygen atom, and can further include double or triple bonds, and can also include heteroatoms. For example, an allyloxy group or a methoxyethoxy group is also an alkoxy group within the meaning herein, as is a methylenedioxy group in a context where two adjacent atoms of a structure are substituted therewith.

The terms “halo,” “halogen,” or “halide” group, as used herein, by themselves or as part of another substituent, mean, unless otherwise stated, a fluorine, chlorine, bromine, or iodine atom.

The term “hydrocarbon” or “hydrocarbyl” as used herein refers to a molecule or functional group that includes carbon and hydrogen atoms. The term can also refer to a molecule or functional group that normally includes both carbon and hydrogen atoms but wherein all the hydrogen atoms are substituted with other functional groups. The term “hydrocarbyl” refers to a functional group derived from a straight chain, branched, or cyclic hydrocarbon, and can be alkyl, alkenyl, alkynyl, aryl, cycloalkyl, acyl, or any combination thereof. Hydrocarbyl groups can be shown as (Cₐ-C_{b})hydrocarbyl, wherein a and b are integers and mean having any of a to b number of carbon atoms. For example, (C₁-C₄)hydrocarbyl means the hydrocarbyl group can be methyl (C₁), ethyl (C₂), propyl (C₃), or butyl (C₄), and (C₀-C_{b})hydrocarbyl means in certain aspects there is no hydrocarbyl group. A hydrocarbylene group is a diradical hydrocarbon, e.g., a hydrocarbon that is bonded at two locations.

The term “number-average molecular weight” (Mₙ) as used herein refers to the ordinary arithmetic mean of the molecular weight of individual molecules in a sample. It is defined as the total weight of all molecules in a sample divided by the total number of molecules in the sample. Experimentally, Mₙ is determined by analyzing a sample divided into molecular weight fractions of species i having nᵢ molecules of molecular weight Mᵢ through the formula Mₙ=ΣMᵢnᵢ/Σnᵢ. The Mₙ can be measured by a variety of well-known methods including gel permeation chromatography, spectroscopic end group analysis, and osmometry. If unspecified, molecular weights of polymers given herein are number-average molecular weights.

The term “weight-average molecular weight” as used herein refers to M_{w}, which is equal to ΣMᵢ²nᵢ/ΣMᵢnᵢ, where nᵢ is the number of molecules of molecular weight Mᵢ. In various examples, the weight-average molecular weight can be determined using light scattering, small angle neutron scattering, X-ray scattering, and sedimentation velocity.

The term “oligomer” as used herein refers to a molecule having an intermediate relative molecular mass, the structure of which essentially includes a small plurality of units derived, actually or conceptually, from molecules of lower relative molecular mass. A molecule having an intermediate relative mass can be a molecule that has properties that vary with the removal of one or a few of the units. The variation in the properties that results from the removal of the one of more units can be a significant variation.

The term “solvent” as used herein refers to a liquid that can dissolve a solid, liquid, or gas. Non-limiting examples of solvents are silicones, organic compounds, water, alcohols, ionic liquids, and supercritical fluids.

As used herein, the term “compound” can refer to specific molecule, or a mixture of molecules, having the defined elements. For example, the compound can be a small molecule, or oligomer, which can have a defined molecular weight and/or chain length. As another example, the compound can be a polymer, which contains a mixture of molecules having a distribution of molecular weights and/or chain lengths. As a further example, a composition containing a compound can, in various aspects, contain at least one molecular structure having the defined elements, or can contain a plurality of molecular structures having the defined elements. For example, in various structures of compound having a chain length, e.g., defined by n, can be defined according to a specific chain length or a can be defined according to a range of chain length.

The term “light” as used herein refers to electromagnetic radiation in and near wavelengths visible by the human eye, and includes ultra-violet (UV) light and infrared light, from about 10 nm to about 300,000 nm wavelength.

The term “independently selected from” as used herein refers to referenced groups being the same, different, or a mixture thereof, unless the context clearly indicates otherwise. Thus, under this definition, the phrase “X¹, X², and X³ are independently selected from noble gases” would include the scenario where, for example, X¹, X², and X³ are all the same, where X¹, X², and X³ are all different, where X¹ and X² are the same but X³ is different, and other analogous permutations.

Herein, when it is designated that a variable in the structure can be “a bond,” the variable can represent a direct bond between the two groups shown as linked to that variable, such as a single bond.

As used herein, the term “polymer” refers to a molecule having at least one repeating unit and can include copolymers.

Salts having a positively charged counterion can include any suitable positively charged counterion. For example, the counterion can be ammonium (NH₄⁺), or an alkali metal such as sodium (Na⁺), potassium (K⁺), or lithium (Li⁺). In some aspects, the counterion can have a positive charge greater than +1, which can in some aspects complex to multiple ionized groups, such as Zn²⁺, Al³⁺, or alkaline earth metals such as Ca²⁺ or Mg²⁺.

Salts having a negatively charged counterion can include any suitable negatively charged counterion. For example, the counterion can be a halide, such as fluoride, chloride, iodide, or bromide. In other examples, the counterion can be nitrate, hydrogen sulfate, dihydrogen phosphate, bicarbonate, nitrite, perchlorate, iodate, chlorate, bromate, chlorite, hypochlorite, hypobromite, cyanide, amide, cyanate, hydroxide, permanganate. The counterion can be a conjugate base of any carboxylic acid, such as acetate or formate. In some aspects, a counterion can have a negative charge greater than −1, which can in some aspects complex to multiple ionized groups, such as oxide, sulfide, nitride, arsenate, phosphate, arsenite, hydrogen phosphate, sulfate, thiosulfate, sulfite, carbonate, chromate, dichromate, peroxide, or oxalate.

The polymers described herein can terminate in any suitable way. In some aspects, the polymers can terminate with an end group that is independently chosen from a suitable polymerization initiator, —H, —OH, a substituted or unsubstituted (C₁-C₂₀)hydrocarbyl (e.g., (C₁-C₁₀)alkyl or (C₆-C₂₀)aryl) interrupted with 0, 1, 2, or 3 groups independently selected from —O—, substituted or unsubstituted —NH—, and —S—, a poly(substituted or unsubstituted (C₁-C₂₀)hydrocarbyloxy), and a poly(substituted or unsubstituted (C₁-C₂₀)hydrocarbylamino).

Methods of Inactivating Enveloped ssRNA Virus.

The present disclosure provides a method of destroying, inactivating, reducing the virality of, or otherwise inhibiting the activity of an enveloped ssRNA virus. The enveloped ssRNA virus can be SARS-CoV-2. The method includes contacting the virus with a polyelectrolyte compound, optionally in the presence of light. For example, the method can involve contacting the virus with a compound having the structure of any of the compounds described herein, such as the structure of Formulas I-V. The method can further involve incubating the virus and the compound together.

For example, a surface or area known or suspected to be contaminated with SARS-CoV-2 virus, or other enveloped ssRNA virus, can be treated with a composition including one or more of the polyelectrolyte compounds described herein so as to contact the virus with the compounds. Treatment can be performed in various ways, such as spraying the compound onto the surface or area, applying a coating containing the compound to the surface, filtering the air in the area through a filter containing the compound, filtering water including the virus through a filter treated with the compound, or wiping the surface down with a substrate containing the compound.

Treatment of the virus serves to inactivate it thus reduce its ability to infect, lyse, or otherwise reduce spread infection.

The polyelectrolyte compound can be provided at a concentration of about 0.01 μg/mL to about 10,000 mg/mL, about 0.1 μg/mL to about 100 mg/mL, about 1 μg/mL to about 100 mg/mL, about 1 μg/mL to about 50 mg/mL, or 10 μg/mL. The concentration can correspond to the concentration of the polyelectrolyte in a solution, composition, or substrate contacting the virus. About 0.01 μg/mL to about 10,000 mg/mL, about 0.1 μg/mL to about 100 mg/mL, about 1 μg/mL to about 100 mg/mL, about 1 μg/mL to about 50 mg/mL, or 10 μg/mL can be provided per 2.5×10⁵ plaque forming units of SARS-CoV-2 virus, or other enveloped ssRNA virus.

Contacting can occur in the solid phase or in liquid media. For example, contacting can occur in solution containing dissolved or suspended compound, or on a solid substrate containing deposited, embedded, or surface-bound compound. The solid substrate can be a hard surface like a non-porous countertop, or it can be a soft, flexible, or porous surface like a cloth mask. Contacting can be accomplished by use of one or more of foamed applicators, cotton swabs, saturated swab sticks, saturated wipes, saturated sponges, aerosols, sprays, brushes, and dips.

The method can further include an incubation period during which the compound and the virus are in contact. During this period, the compound in the presence of UV or visible light can act upon the virus to destroy, inactivate, reduce the virality of, or otherwise inhibiting the virus. This incubation period can be about or at least 5 minutes, 10 minutes, 15 minutes, 20 minutes, 30 minutes, 40 minutes, 50 minutes, 1 hour, 2 hours, 3 hours, 5 hours, 10 hours, or at least or about 1 day. Incubation of the virus and compounds together in the presence of light can serve to inactivate the virus to a greater extent than light alone, compound alone, or virus alone, for the same period of time.

The incubation period can further involve treatment with UV or visible light. The light can be UV light, e.g., UVA, UVB and/or UVC. For example, light in the range of 200-280 nm, 280-315 nm, and/or 315-380 nm. The light can be visible light, e.g., light in the range of 380 nm to 740 nm. In various aspects, the light is daylight, conventional indoor lighting, or a black light.

The incubation period can further involve contacting the virus and compound together in liquid media. The liquid media can be an aqueous solution or suspension. In other aspects, the liquid media can be moisture on a substrate, e.g., moisture provided by a cleaning solution deposited on the substrate surface. The liquid media can be provided by wetting or spraying the surface or area to be treated. The liquid media can be aerosolized droplets.

The incubation period can further involve heating the virus and compound together at a temperature above room temperature. For example, the virus and compound can be heated together at temperature at or above 30° C., 31° C., 32° C., 33° C., 34° C., 35° C., 36° C., 37° C., 38° C., 39° C., or at or above 40° C.

Ideally, treatment of the virus and incubation period will occur prior to human contact with the virus, for example, about or at least 5 minutes, 10 minutes, 15 minutes, 20 minutes, 30 minutes, 40 minutes, 50 minutes, 1 hour, 2 hours, 3 hours, 5 hours, 10 hours, or at least or about 1 day prior. Treatment of the virus, including the incubation period, can inhibit cytopathic effects of SARS-CoV-2 by at least or about 50%, 80%, 90%, 95%, 99%, 99.9%, or at least or about 99.99%. The treatment of the virus, including the incubation period, can destroy or inactivate 50%, 80%, 90%, 95%, 99%, 99.9% or at least or about 99.99% of SARS-CoV-2. Such effects can be achieved in about or at least 5 minutes, 10 minutes, 15 minutes, 20 minutes, 30 minutes, 40 minutes, 50 minutes, 1 hour, 2 hours, 3 hours, 5 hours, 10 hours, or at least or about 1 day of incubation of the virus and compound together, such as in the presence of light.

In various aspects, the present disclosure also provides a method of reducing SARS-CoV-2, or other enveloped ssRNA virus, viability on a substrate. The method can involve treating a substrate with a polyelectrolyte compound, such as a compound having the structure of Formula I-Formula V, optionally in the presence of light. For example, the surface of the substrate can be treated with the polyelectrolyte compound, or the polyelectrolyte can be embedded within the substrate. The substrate can be a fabric, a work surface, a medical device, packaging materials, personal protective equipment, a fibrous support, a wipe, a water filter, an air filter, a mask, or a combination thereof. The wipe can be any suitable wipe, such as a Kimwipe®, a Diamond Wipe® substrate, and the like. The fibrous support can be any suitable fibrous support, such as a filter (e.g., for air and/or water filtration), a HYDAC glass fiber support, and the like. In various aspects, the polyelectrolyte compound can retain active against bacteria and viruses after more than one use of the substrate; for example, a wipe can be used more than once, or an air or water filter can be used many times before replacement is needed.

The substrate can be incubated, and the compounds together in the presence of light can serve to inactivate the virus to a greater extent than light alone, compound alone, or virus alone, for the same period of time. In various aspects, the substrate is in contact with SARS-CoV-2, or expected to come in contact with SARS-CoV-2.

The substrate can be personal protective equipment, such as a gown, scrub, glove, hair cover, face mask, sleeve, cuff, respirator, face shield, glasses, goggles, or apron. The substrate can also be substrate is a wipe, a tissue, a bandage, a medical device, a surgical instrument, a sponge, a textile, a diaper, a counter-top, a food preparation surface, a wound dressing, a dressing for surgical incisions, a keyboard surface, a packing for wounds, a packing for surgical incisions, a nasal packing, and a feminine care product. The substrate can be a device used for aerosol-generating procedures, for example endotracheal intubation.

In various aspects, the substrate containing the polyelectrolyte compound is incubated with light for a period after confirmed or suspected contamination with SARS-CoV-2.

The present disclosure yet also provides a method of reducing the ability of SARS-CoV-2 to spread via a surface, including treating a surface of an object with a polyelectrolyte compound, such as a compound having the structure of Formula I-Formula V, optionally in the presence of light. The object can be fabric, a work surface, a medical device, packaging materials, personal protective equipment, a fibrous support, a wipe, a water filter, an air filter, a mask, office supplies, office equipment, medical supplies, medical equipment, utensils, eating surfaces, drinking glasses, handles, knobs, railings, or other objects for which their use may be shared between multiple people. The surface can be treated prior to providing the object to a human being for using, donning, or handling it.

The present disclosure further provides a method of destroying, inactivating, reducing the virality of, or otherwise inhibiting the activity of SARS-CoV-2, or other enveloped ssRNA virus, in circulated air. As such, the method can be used to inactivate SARS-CoV-2 in recirculated indoor air, such as in schools, offices, classrooms, meeting rooms, hospitals, houses of worship, bars, restaurants, retail stores, malls, and commercial establishments. The present disclosure further provides a method of destroying, inactivating, reducing the virality of, or otherwise inhibiting the activity of SARS-CoV-2, or other enveloped ssRNA virus, in water. As such, the method can be used to inactive SARS-CoV in water.

The method can include contacting a filter with a polyelectrolyte compound, such as a compound having the structure of Formula I-Formula V; and exposing the filter to SARS-CoV-2 from circulated air (e.g., in the presence of light or in the dark). The method can include contacting a filter with a polyelectrolyte compound, such as a compound having the structure of Formula I-Formula V; and exposing the filter to SARS-CoV-2 from water (e.g., in the presence of light or in the dark).

Air or water filters of the present invention can be made of any suitable material. In some examples, the filter can be a glass fiber filter.

In various aspects, the filter can have a pore size of less than or about 0.05 micron, 0.06 micron, 0.07 micron, 0.08 micron, 0.09 micron, 0.1 micron, 0.11 micron, 0.12 micron, 0.13 micron, 0.14 micron, 0.15 micron, 0.16 micron, 0.17 micron, 0.18 micron, 0.19 micron, 0.2 micron, 0.3 micron, 0.4 micron, or 0.5 micron.

The method can further involve incubating the virus and the compound together, for example, by heating the filter at a temperature of 35° C. for a period of at least 60 minutes. For example, the virus and compound can be heated together at temperature at or above 30° C., 31° C., 32° C., 33° C., 34° C., 35° C., 36° C., 37° C., 38° C., 39° C., or at or above 40° C. This incubation period can be about or at least 5 minutes, 10 minutes, 15 minutes, 20 minutes, 30 minutes, 40 minutes, 50 minutes, 1 hour, 2 hours, 3 hours, 5 hours, 10 hours, or at least or about 1 day. The incubation period for the filter can correspond to a period of recirculated air flow, can correspond to a time period during which virus is captured in the filter, or both.

The method can further include irradiating the virus and a coated filter together (e.g., water filter or air filter) with light, such as with visible light, ambient light, near-UV light, or a combination thereof. Such a method can allow the anti-viral effects to be turned on or off on demand. In other aspects, the filter can be effective to destroy, inactivate, reduce the virality of, or otherwise inhibit the activity of the virus in the dark.

In various aspects of the method, an area is treated with the disinfectant composition via a spray, a lotion, a dip, a bath, or by contact with a substrate saturated with the disinfectant composition.

Without intending to be limited by theory, it appears that the surface structure of SARS-CoV-2 has anionic and cationic surface patches that are amenable to attractive interactions with correspondingly charged polyelectrolytes. Surprisingly, the surface association between SARS-CoV-2 and the presently described polyelectrolytes appears to be close and prolonged enough that radical degradation of the virus can be imposed in the presence of photoactivation.

### Polyelectrolyte Compounds.

Various polyelectrolyte compounds can be used in the presently described methods. For example, polyelectrolyte compounds can include oligomeric polyelectrolytes, polymeric polyelectrolytes, polyelectrolytes containing negatively-charged pendant groups, and polyelectrolytes containing positively-charged pendant groups. The polyelectrolyte compounds can be negatively-charged polyelectrolytes, positively-charged polyelectrolytes, or polyampholyte, which contain both negatively-charged groups and positively-charged charged groups. For example, in some aspects, the polyelectrolyte may contain only neutral or positively-charged functional groups, while in other aspects the polyelectrolyte may contain only neutral or negatively-charged functional groups. In various aspects the polyelectrolyte is negatively-charged or positively-charged functional groups are attached to an internal repeating unit, or they are attached at a terminal group.

For example, the present disclosure provides a composition including a compound having the structure of Formula I or Formula II:

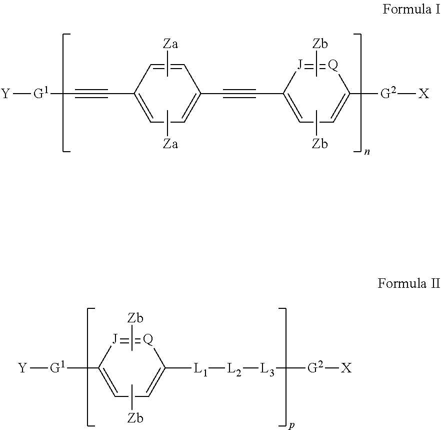

wherein

each of X and Y is independently H, COOR, O—(CH₂)ₘ-T, NH₂, or COR;

each of Za and Zb is independently H, O—(CH₂)ₘ-T, O—C₂H₄—(OCH₂)ₘ—R;

each of G¹ and G² is independently a bond, C₂C₆H₄, C₆H₄, C₂C₄S, or C₄S;

J and Q are each C or CH so as to provide a benzene ring, or J and Q are together S so as to provide a thiophene ring;

n is 1 to 200;

p is 1 to 10,000;

m is 0 to 10;

each of R is independently methyl, ethyl, n-propyl, isopropyl, phenyl, t-butyl, isobutyl, n-butyl, pentyl, hexyl, heptyl, octyl, cyclopropyl, cyclobutyl, cyclopentyl, cyclyhexyl, methoxymethyl, or trifluoromethyl;

each of T is independently H, SO₃⁻, COO⁻, COOR, DABCO, N-alkyl DABCO, imidazolyl, N-alkyl imidazolyl, NR₂, NHR₂⁺, or NR₃⁺;

L1 is independently a bond or —C≡C—;

L2 is independently a bond, a substituted or unsubstituted phenylene, thiophenylene, azulenylene, heptalenylene, biphenylene, indacenylene, fluorenylene, phenanthrenylene, triphenylenylene, pyrenylene, naphthacenylene, chrysenylene, biphenylenylene, anthracenylene, and naphthylene;

L3 is independently a bond or —C≡C—; and

at least one occurrence of Y, X, Za, and Zb is independently O—(CH₂)ₘ-T.

In various aspects, the compound has the structure of formula I and n=1-3, or n=1. For example, the compound can be an oligomer. The compound can have a molecular weight of less than or about 500 Da, 1000 Da, 2000 Da, 3000 Da, 4000 Da, or less than or about 5000 Da.

In various examples, the compound can have each of X and Y is independently H or COOR. Each of Za can be O—(CH₂)ₘ-T, G¹ can be C₆H₄, G² can be a bond, J and Q can be each CH so as to provide a benzene ring, n can be 1, p can be 1 to 10,000, m can be 2 to 3, each of R can be methyl or ethyl, each of T can be SO₃⁻, N-hexyl DABCO, or N-methyl imidazolyl, L1 can be —C≡C—, L2 can be unsubstituted phenylene, and L3 can be —C≡C—. As a further example, p can be about, at least, or at most, 5, 10, 50, 100, 200, 300, 400, 500, 600, 700, 800, 900, 1000, 2000, 3000, 4000, 5000, 6000, 7000, 8000, 9000, or at least or about 10000.

In various examples, the compound can have the structure of Formula I and each of X and Y can be O—(CH₂)ₘ-T, each of Za is H, G can be C₆H₄, G² can be a bond, J and Q are each CH so as to provide a benzene ring, n can be 1, m can be 3, each of R can be independently methyl or ethyl, and each of T can be independently N-alkyl imidazolyl. In various aspects, each of Za can be a negatively charged functional group, a positively charged functional group, a neutral functional group, or a mixture thereof. Each of Zb can be a negatively charged functional group, a positively charged functional group, a neutral functional group, or a mixture thereof. Each of X can be a negatively charged functional group, a positively charged functional group, a neutral functional group, or a mixture thereof. Each of Y can be a negatively charged functional group, a positively charged functional group, a neutral functional group, or a mixture thereof.

In various further examples, the compound can have the structure of Formula I and each of X and Y can be independently H or COOR; each of Za can be O—(CH₂)ₘ-T; G¹ can be C₆H₄; G² can be a bond; J and Q are each CH so as to provide a benzene ring; n can be 1; m can be 3; each of R can be ethyl; and each of T can be SO₃⁻.

In various further examples, the compound can have two, or more, O—(CH₂)ₘ-T groups. T can be any of various charged functional groups, including SO₃⁻, n-alkylimidazolium, n-alkyl DABCO. The n-alkyl groups can be methyl, ethyl, propyl, butyl, pentyl, hexyl, heptyl, octyl, and the like. In various aspects, m can be 1, 2, 3, 4, 5, 6, 7, 8, 9, or 10, for example, 2 to 6 or 3 to 7. In some examples, the compound can contain a total of three benzene rings, two ethynylene linkages, or both.

In various aspects, the compound has a maximum absorbance between 250 nm and 700 nm at less than about 380 nm. For example, the maximum absorbance between 250 nm and 700 nm can be less than or about 280, 290, 300, 310, 320, 330, 340, 350, 360, 370, 380, 390, or less than or about 400.

In further aspects, the compound, or composition, has a maximum absorbance between 250 nm and 700 nm of less than about 1.2 A. For example, the compound can have a maximum absorbance between 250 nm and 700 nm of less than about 0.5 A, 0.6 A, 0.7 A, 0.8 A, 0.9 A, 1.0 A, 1.1 A, or 1.2 A. Absorbance can be provided by a Lambda 35 U/Vis spectrometer from PerkinElmer (Waltham, Mass.), the user manual for which is incorporated by reference herewith in its entirety.

As another example, the compound can have the structure of Formula III:

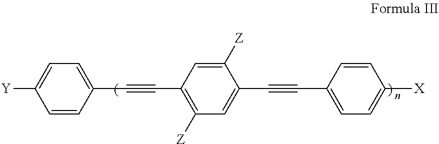

wherein

X can be COOEt, Y can be H, Z can be OCH₂CH₂CH₂N(CH₃)₃⁺, and n can be selected from the group consisting of numbers between 1 and 10; or

X can be Y and can be selected from the group consisting of H, CO₂Et, COO⁻, NH₂ and COCH₃, Z can be OCH₂CH₂CH₂N(CH₃)₃⁺, and n can be selected from the group consisting of numbers between 1 and 10; or

X can be Y═OCH₂CH₂CH₂N(CH₃)₃⁺, Z can be H, and n can be 1.

The compound can also have the structure of Formula IV:

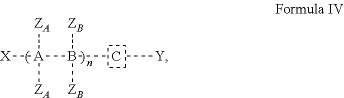

In various aspects, in the compound of Formula IV has:

n is selected from the group consisting of whole numbers between 5 and 200;

A is C₂C₆H₂;

B is selected from the group consisting of C₂C₆H₂ and C₂C₄S;

C is either C₆H₄ or not present;

X is selected from the group consisting of H, [C₂C₆H₄]₂COOCH₂CH₃, and [C₂C₄H₂S][C₂C₆H₄]COOCH₂CH₃;

Y is selected from the group consisting of H and COOCH₂CH₃;

Z_{A} is selected from the group consisting of O(CH₂)ₖ(C₆H₁₂N₂)C₆H₁₃²⁺, O(CH₂)ₖSO₃⁻, O(CH₂)ₖN(CH₂CH₃)₃⁺, and O(CH₂)ₖN(CH₃)₃⁺, wherein k is selected from the group consisting of the whole numbers between 1 and 10;

Z_{B} is selected from the group consisting of H and (OCH₂CH₂)₃OCH₃;

wherein, it ZA is O(CH₂)ₖ(C₆H₁₂N₂)C₆H₁₃²⁺, then Z_{B} is H, A=C₂C₆H₂, C, if present, is C₆H₄, and X is selected from the group consisting of H and [C₂C₆H₄]₂COOCH₂CH₃;

if Y is H, then X is not H;

if X is H, then Y is not H, and C is not present;

if X is [C₂C₆H₄]₂COOCH₂CH₃, then Y is COOCH₂CH₃ and C is C₆H₄;

if Z_{A} is O(CH₂)ₖ—SO₃⁻, then Z_{B} is H, A=C₂C₆H₂, C is not present, and one but not both of X or Y is H;

if Z_{A} is O(CH₂)ₖN(CH₂CH₃)₃⁺, then Z_{B} is (OCH₂CH₂)₃OCH₃, A=C₂C₆H₂, C is not present, and one but not both of X or Y is H;

if Z_{A} is O(CH₂)ₖN(CH₃)₃⁺, and Z_{B} is (OCH₂CH₂)₃OCH₃, then C is not present, A=C₂C₆H₂ and one but not both of X or Y is H;

if Z_{A} is O(CH₂)ₖN(CH₃)₃⁺, Z_{B} is H, and C is not present, then A=C₂C₄H₂S and one but not both of X or Y is H;

if Z_{A} is O(CH₂)ₖN(CH₃)₃+, Za is H, and C is present, then Y═COOCH₂CH₃ and X is selected from the group consisting of [C₂C₆H₄]₂COOCH₂CH₃ and [C₂C₄H₂S][C₂C₆H₄]COOCH₂CH₃; and

if X is [C₂C₆H_{4]2}COOCH₂CH₃, then B═C₂C₆H₂;

if X is [C₂C₄H₂S][C₂C₆H₄]COOCH₂CH₃, then B═C₂C₄S.

In various aspects, in the compound of Formula IV has:

n is selected from the group consisting of whole numbers between 5 and 200;

A is C₂C₆H₂;

B is selected from the group consisting of C₂C₆H₂ and C₂C₄S;

C is either C₆H₄ or not present;

X is selected from the group consisting of [C₂C₆H₄]₂COOCH₂CH₃ and [C₂C₄H₂S][C₂C₆H₄]COOCH₂CH₃;

Y is COOCH₂CH₃;

Z_{A} is selected from the group consisting of O(CH₂)ₖ(C₆H₁₂N₂)C₆H₁₃²⁺, O(CH₂)ₖSO₃⁻, and O(CH₂)ₖN(CH₃)₃⁺; where k is selected from the group consisting of the whole number between 1 and 10;

Z_{B} is H;

if Z_{A} is O(CH₂) (C₆H₁₂N₂)C₆H₁₃²⁺, then A=C₂C₆H₂, C is C₆H₄, Y is COOCH₂CH₃, and X is [C₂C₆H₄]₂COOCH₂CH₃;

if Z_{A} is O(CH₂)ₖN(CH₃)₃⁺ and C is present, then Y═COOCH₂CH₃ and X is selected from the group consisting of [C₂C₆H₄]₂COOCH₂CH₃ and [C₂C₄H₂S][C₂C₆H₄]COOCH₂CH₃;

if B═C₂C₆H₂, then X is [C₂C₆H_{4]2}COOCH₂CH₃; and

if B═C₂C₄S, then X is [C₂C₄H₂S][C₂C₆H₄]COOCH₂CH₃.

In various aspects, in the compound of Formula IV has:

n is selected from the group consisting of 1, 2, 3 and 4;

A is selected from the group consisting of C₂C₆H₂ and C₂C₄S;

B═C₂C₆H₂;

C is either C₆H₄ or not present;

X is selected from the group consisting of COOCH₂CH₃, O(CH₂)ₖN(CH₃)₃⁺, O(CH₂)ₖSO₃⁻, and O(CH₂)ₖ(C₆H₁₂N₂)C₆H₁₃;

Y is selected from the group consisting of COOCH₂CH₃, O(CH₂)ₖN(CH₃)₃⁺, O(CH₂)ₖSO₃⁻, C₆H₂(OCH₃)₃, and O(CH₂)ₖ(C₆H₁₂N₂)C₆H₁₃²⁺;

k is selected from the group of whole numbers from 1 to 10;

Z_{A} is selected from the group consisting of H and O(CH₂)ⱼ(C₆H₁₂N₂)C₆H₁₃²⁺, wherein j is selected from the group of whole numbers from 1 to 10; and

Z_{B}═H;

wherein:

if X═COOCH₂CH₃ and Y═COOCH₂CH₃, then A=B═C₂C₆H₂, C is C₆H₄, and Z_{A} is O(CH₂)ₖ(C₅H₁₂N₂)C₆H₁₃²⁺;

if Z_{A} is H, A is C₂C₆H₂, X═O(CH₂)ₖN(CH₃)₃⁺, and C═C₆H₄, then Y is selected from the group consisting of COOCH₂CH₃, O(CH₂)ₖSO₃⁻, C₆H₂(OCH₃)₃, and O(CH₂)ₖ(C₆H₁₂N₂)C₆H₁₃²⁺;

if Z_{A} is H, A is C₂C₆H₂, X═O(CH₂)ₖN(CH₃)₃⁺, C═C₆H₄, and B═C₂C₆H₂, then k is selected from the group consisting of 1, 2, 4, 5, 6, 7, 8, 9, and 10;

if Z_{A} is H, A is C₂C₆H₂, X═O(CH₂)ₖN(CH₃)₃⁺, and C is not present, then Y═C₆H₂(OCH₃)₃;

if Z_{A} is H, A is C₂C₆H₂ and X═O(CH₂)ₖSO₃⁻, then C═C₆H₄ and Y═O(CH₂)ₖSO₃⁻;

if Z_{A} is H, A is C₂C₆H₂, and X═O(CH₂)ₖ(C₅H₁₂N₂)C₆H₁₃²⁺, then C═C₆H₄ and Y═O(CH₂)ₖ(C₆H₁₂N₂)C₆H₁₃²⁺;

if A is C₂C₄S, then C═C₆H₄ and X is selected from the group consisting of O(CH₂)ₖN(CH₃)₃⁺ and O(CH₂)ₖ(C₆H₁₂N₂)C₆H₁₃²⁺;

if X is O(CH₂)ₖ(C₆H₁₂N₂)C₆H₁₃²⁺, then Y═O(CH₂)ₖ(C₆H₁₂N₂)C₆H₁₃²⁺.

In various aspects, in the compound of Formula IV the poly(phenylene ethynylene) is grafted to a substrate by chemisorption or physisorption.

In further aspects, the compound can have the structure of Formula V:

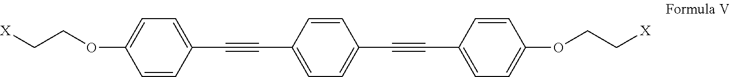

wherein X is n-hexyl DABCO, n-methyl imidazolium, or trimethylammonium. For example, the compound can be:

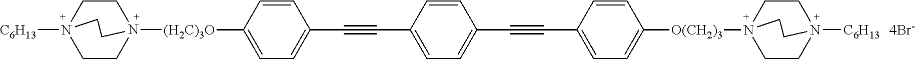

In various further aspects, the composition can include a compound according to Compound A, Compound B, Compound C, Compound D, or optionally substituted derivatives thereof. In various aspects, the composition can include a compound according to Compound E. In various aspects, the composition can include a compound according to Formula VII-X.

Compound A has the structure:

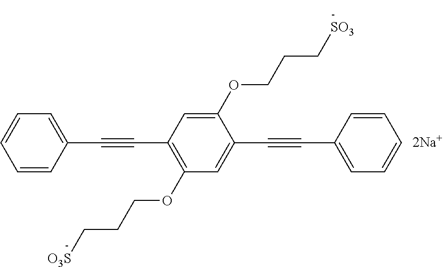

Compound B has the structure:

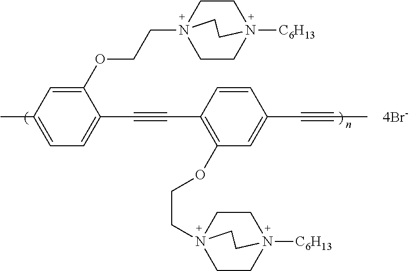

Compound C has the structure:

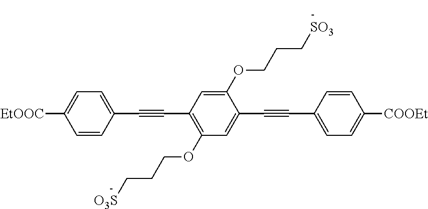

Compound D has the structure:

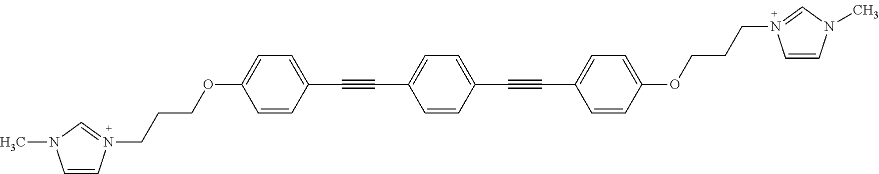

Compound E has the structure:

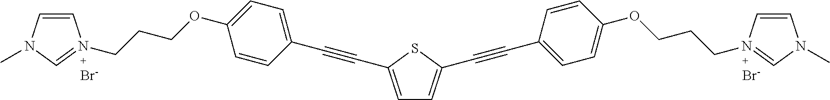

Formula VII has the structure:

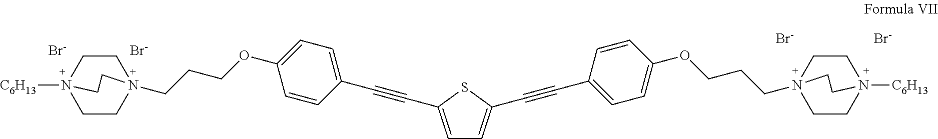

Formula VIII has the structure:

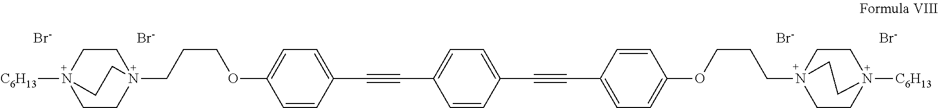

Formula IX has the structure:

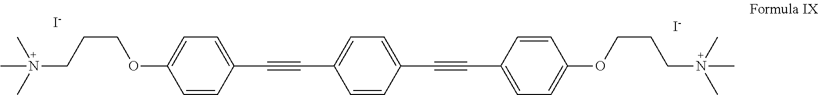

Formula X has the structure:

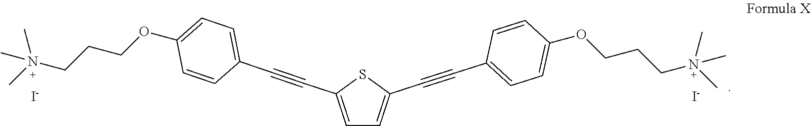

In various aspects, the compound contains three phenyl groups and two ethynylene groups. For example, the compound contains a phenylene-ethynylene-phenylene-ethynylene-phenylene core.

In further examples, the compound is a polythiophene. For example, the polythiophene can have the structure:

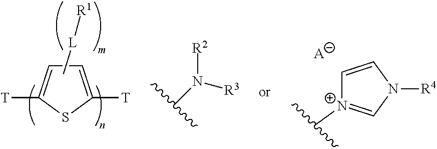

Each of R2 and R3 can be independently C1-C6 alkyl or R2 and R3 taken together are linked C2-C5 alkyl which forms a 3 to 6-membered saturated heterocyclic ring together with the nitrogen at which they attach.

R4 can be C1-C6 alkyl; A can be a counterion; L can be a divalent C1-C20 alkyl linker, optionally interrupted by 1, 2 or 3 oxygen, sulfur or nitrogen atoms; T can be a terminal group; m can be 1-2; n can be 30 to 120; the polythiophene has a number average molecular weight (Mn) from 10,000 to 40,000; and the polythiophene has a polydispersity index (PDI) is from 1 to 1.3.

For example, the compound can be:

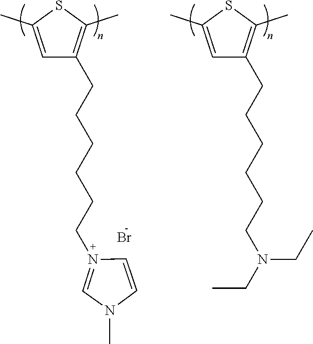

In various further aspects, the compound can be:

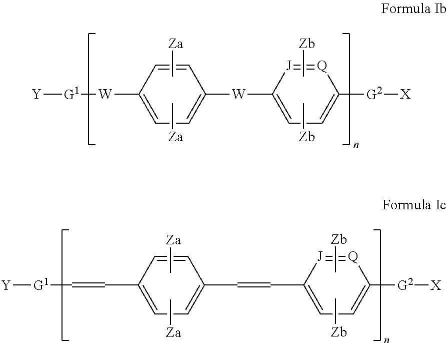

wherein

each of X and Y is independently H, COOR, O—(CH₂)ₘ-T, NH₂, or COR;

each of Za and Zb is independently H, O—(CH₂)ₘ-T, O—C₂H₄—(OCH₂)ₘ—R;

each of G¹ and G² is independently a bond, C₂C₆H₄, C₆H₄, C₂C₄S, or C₄S;

J and Q are each C or CH so as to provide a benzene ring, or J and Q are together S so as to provide a thiophene ring;

n is 1 to 200;

p is 1 to 10,000;

m is 0 to 10;

each of R is independently methyl, ethyl, n-propyl, isopropyl, phenyl, t-butyl, isobutyl, n-butyl, pentyl, hexyl, heptyl, octyl, cyclopropyl, cyclobutyl, cyclopentyl, cyclyhexyl, methoxymethyl, or trifluoromethyl;

each of T is independently H, SO₃⁻, COO⁻, COOR, DABCO, N-alkyl DABCO, imidazolyl, N-alkyl imidazolyl, NR₂, NHR₂⁺, or NR₃⁺;

each of W, if present, can independently be a bond, —C≡C—, or —C≡C—; and

at least one occurrence of Y, X, Za, and Zb is independently O—(CH₂)ₘ-T.

Compounds of Formula Ib and Ic include arylene vinylene compounds, which can have cis double bonds, trans double bonds, or both. For example, the compound can have the structure:

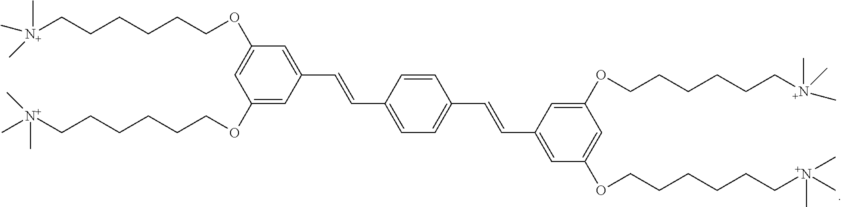

As another example, the compound can have the structure:

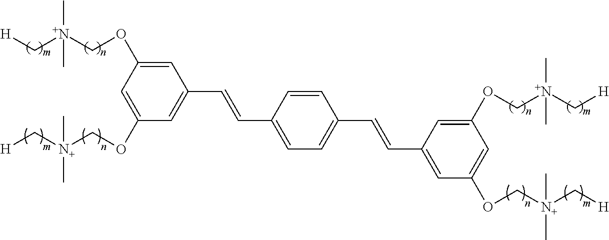

wherein m and n are each independently 1 to 10.

In various further aspects, the compound is a polyelectrolyte selected from the group consisting of:

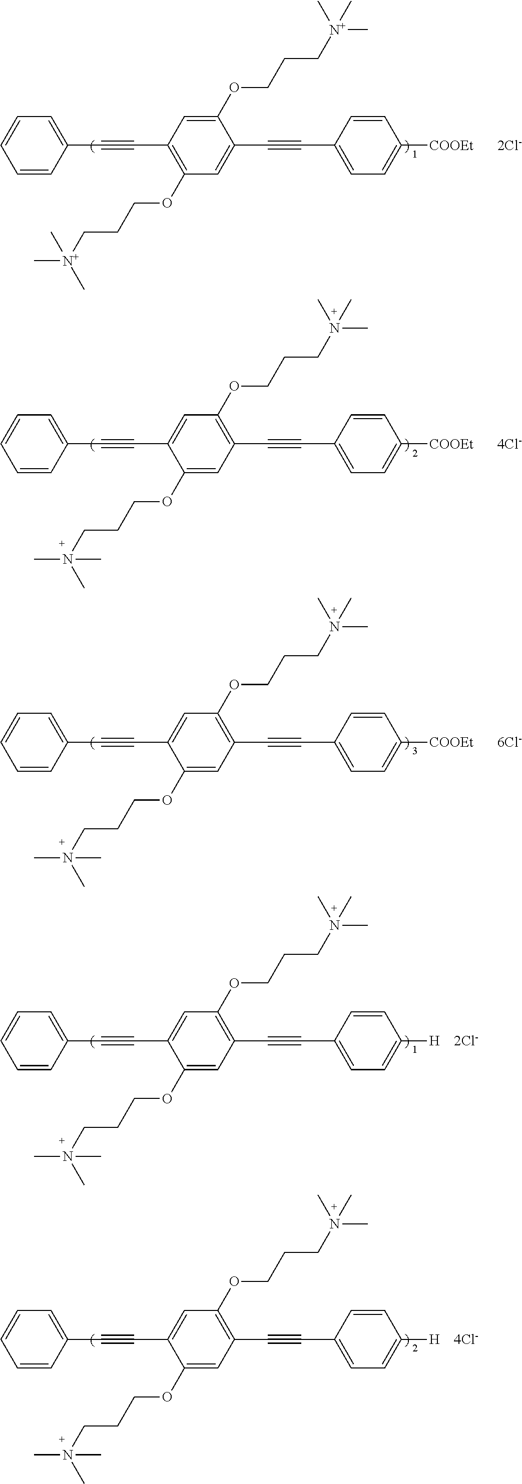

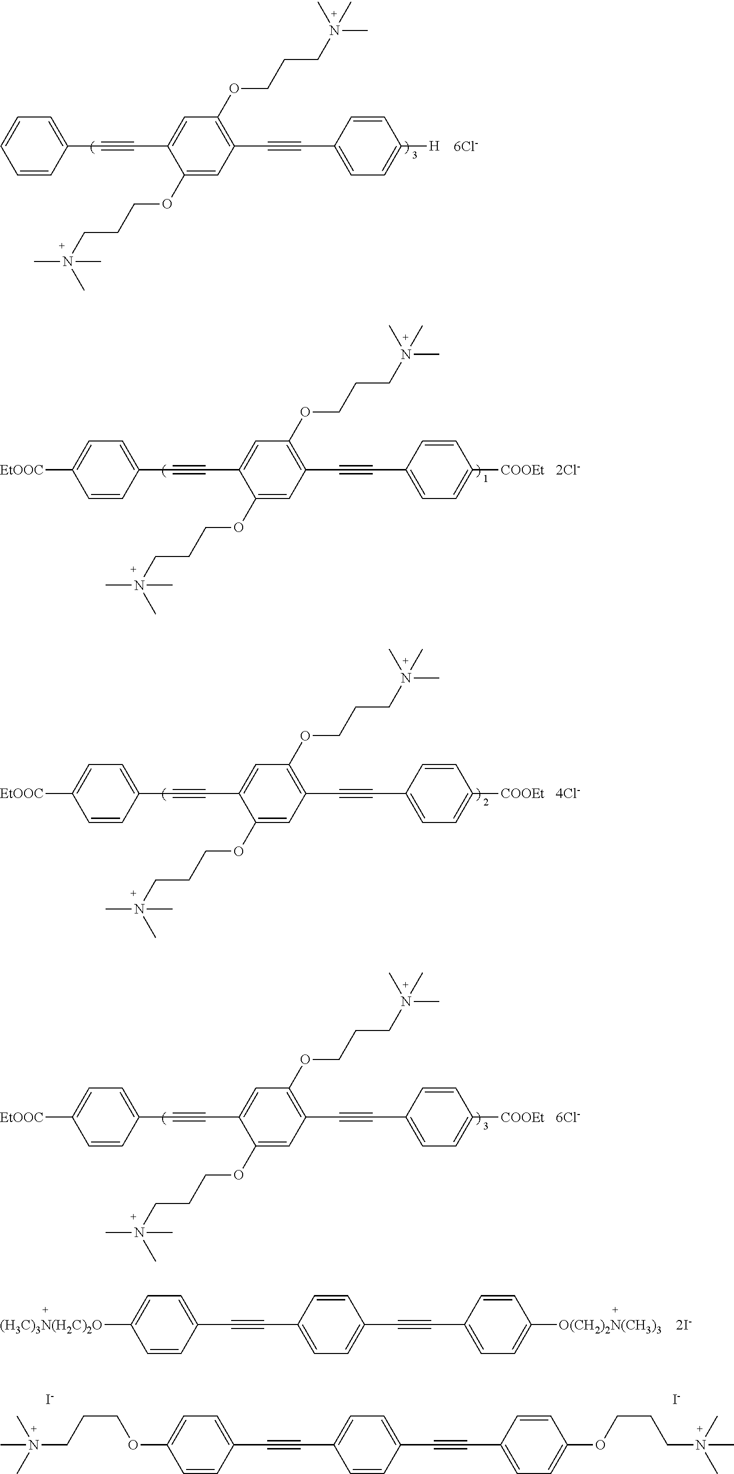

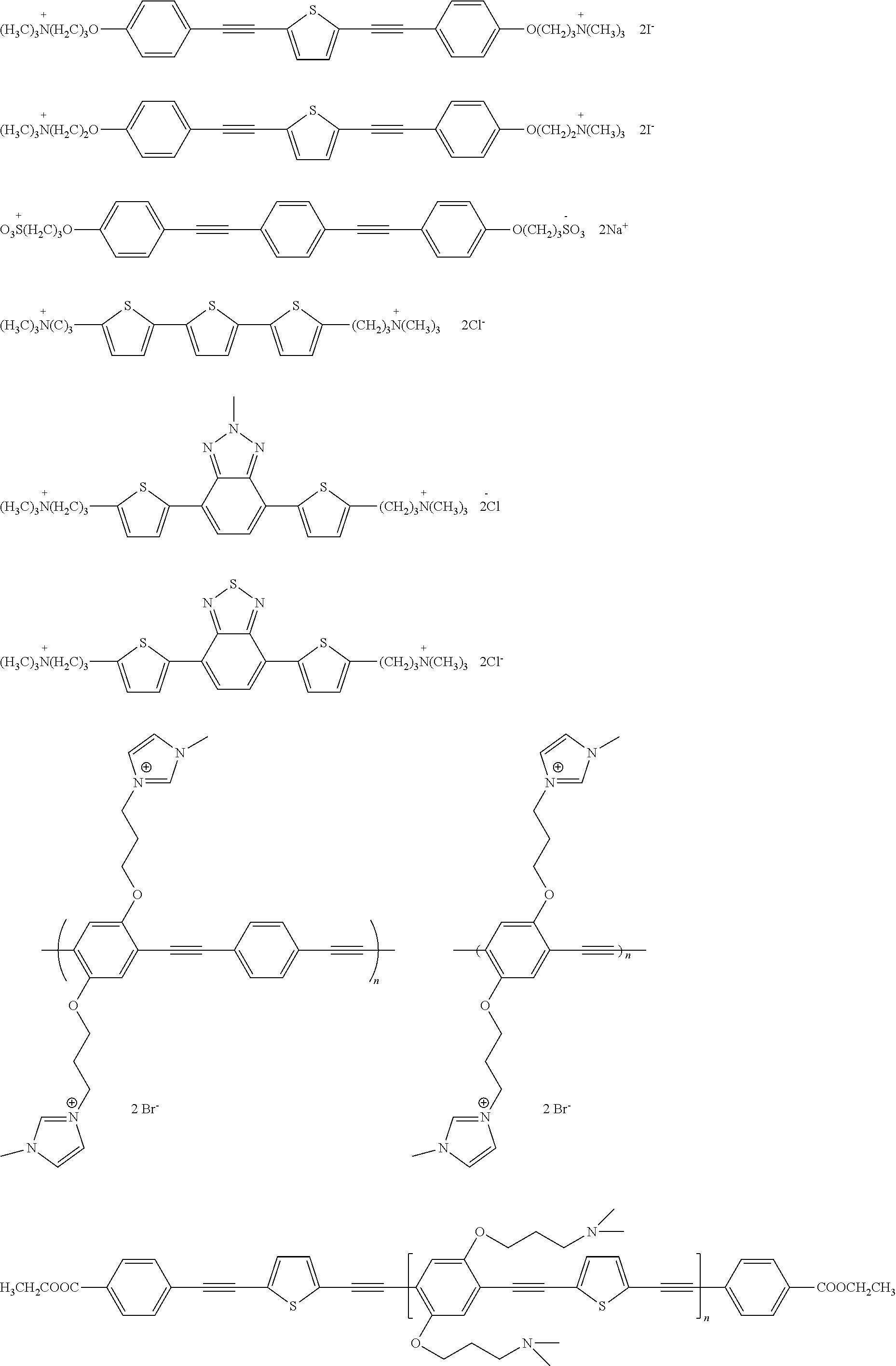

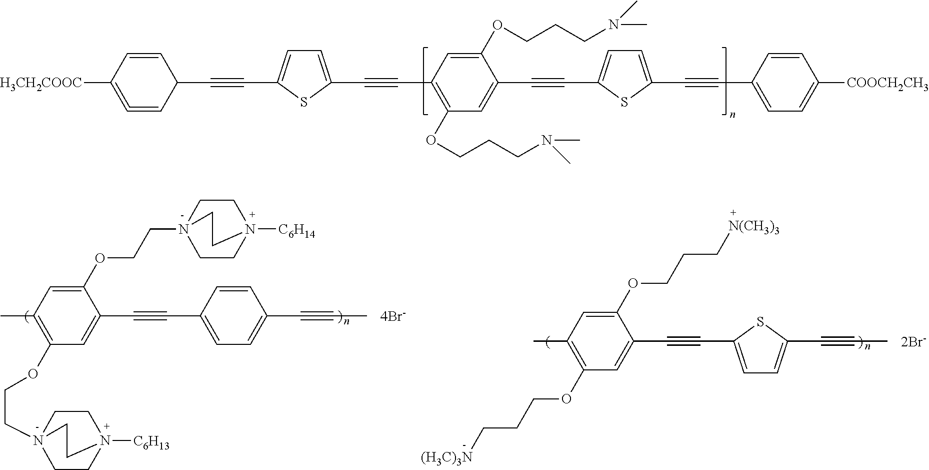

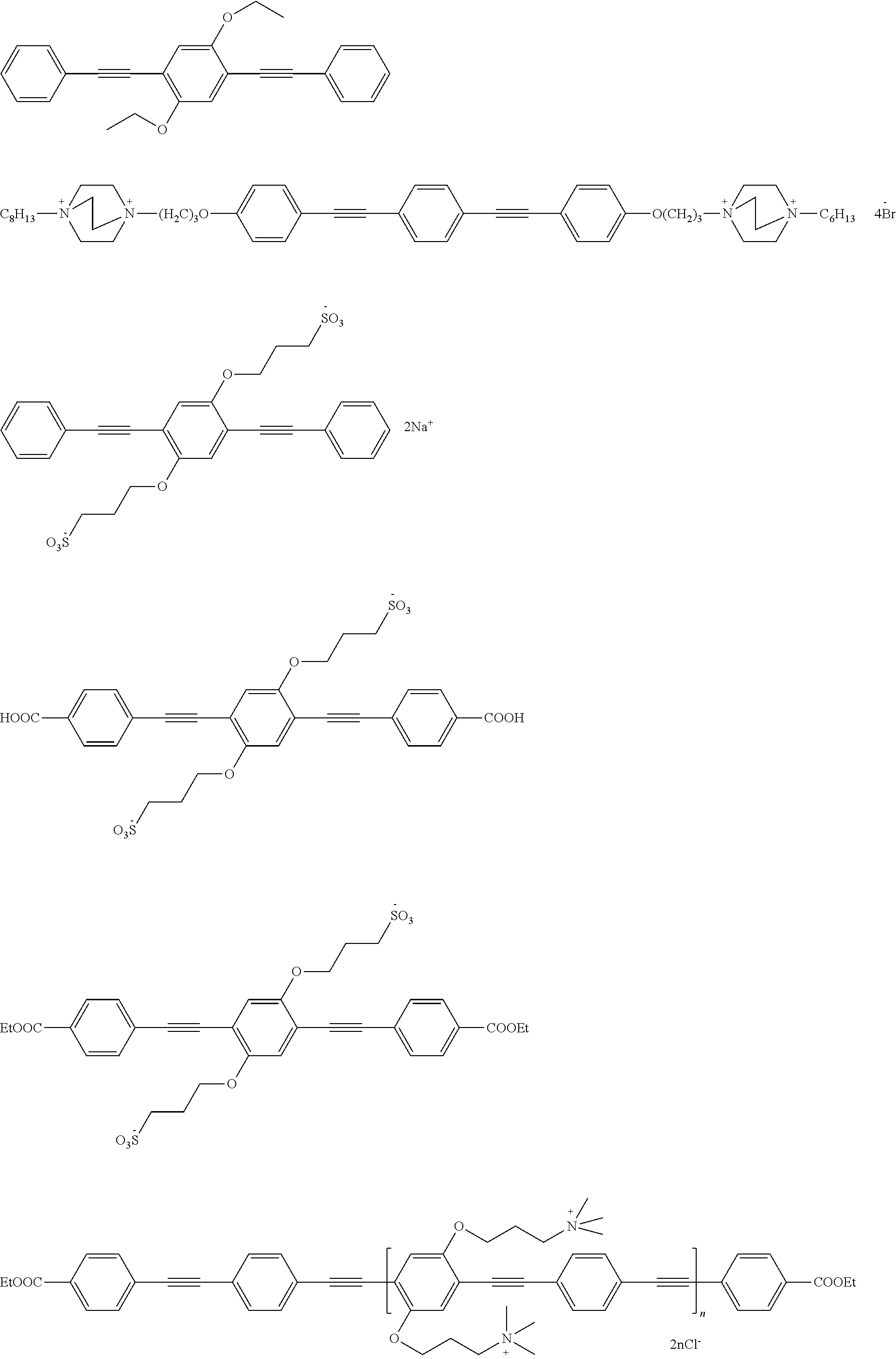

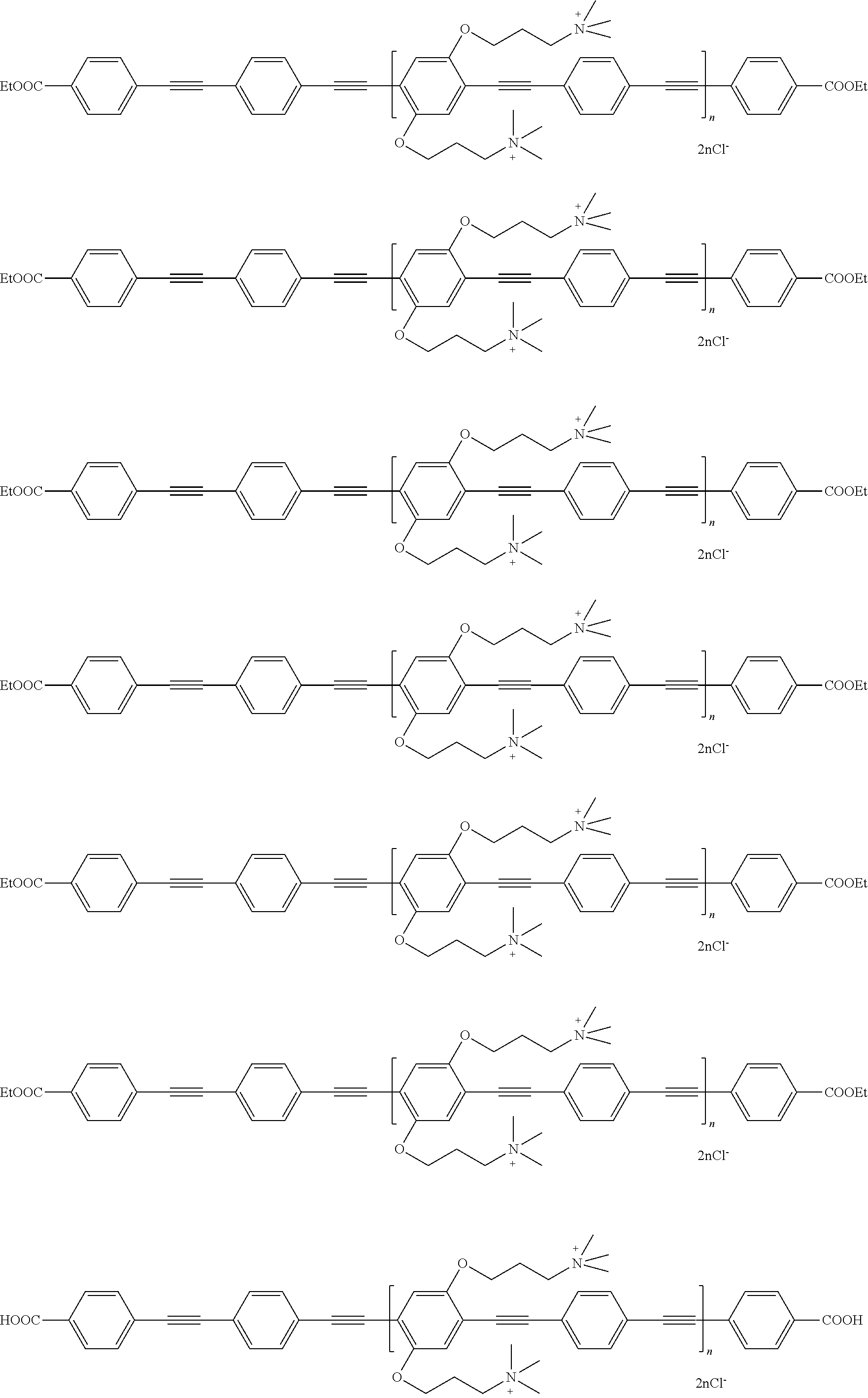

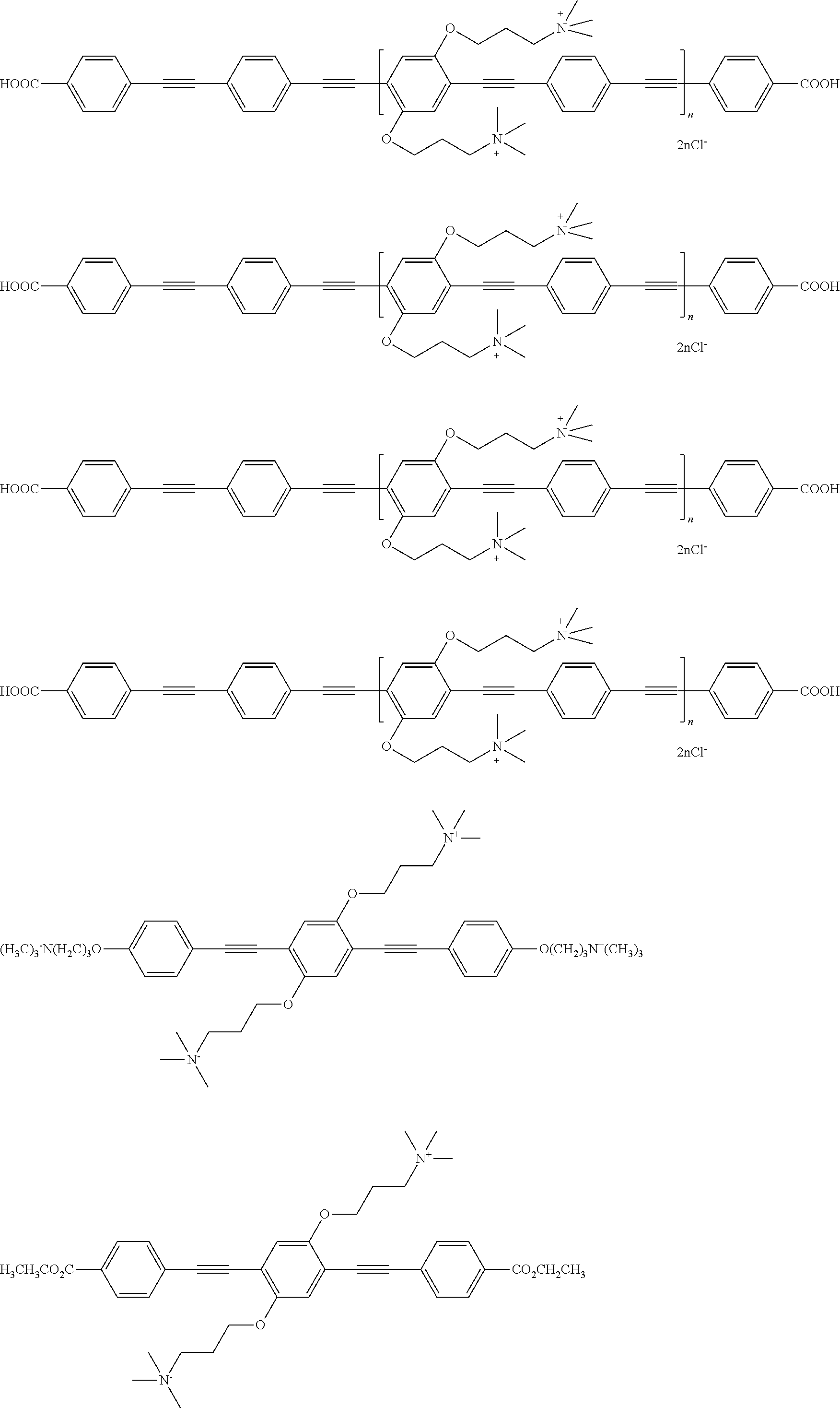

Other suitable polyelectrolytes include those described in U.S. Pat. Nos. 8,598,053, 8,618,009, 8,753,570, 9,005,540, 9,527,806, 9,750,250, 9,968,698, 10,092,000, 10,058,099, 10,174,042, 10,533,991, and PCT International Application Publication No. WO 2019/060586 A1, each of which is incorporated by reference herewith in their entirety.

In various aspects, the compound is a polymer. The polymer can be a mixture of compounds according to the presently described structures, for example, having a distribution of sizes. In various aspects, the composition, or polymer, can have a number average molecular weight (Mn) from 1,000 to 40,000, 1,000 to 4,000, 10,000 to 40,000, or 20,000 to 40,000. In further aspects, the composition, or polymer, can have a polydispersity index (PDI) from 1 to 1.3.

In various aspects, the number average molecular weight (Mn) can be between 10,000 to 40,000. In various further aspects, number average molecular weight (Mn) can be between 10,000 to 15,000, 10,000 to 20,000, 10,000 to 25,000, 10,000 to 30,000, 10,000 to 35,000, 10,000 to 40,000, 20,000 to 25,000, 20,000 to 30,000, 20,000 to 35,000, or 20,000 to 40,000. The number average molecular weight (Mn) can be greater than or about, 6,000, 7,000, 8,000, 9,000, 10,000, 11,000, 12,000, 13,000, 14,000, 15,000, 16,000, 17,000, 18,000, 19,000, 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000, or 45,000 or greater. In various aspects, the number average molecular weight (Mn) can be less than 20,000, 21,000, 22,000, 23,000, 24,000, 25,000, 26,000, 27,000, 28,000, 29,000, 30,000, 31,000, 32,000, 33,000, 34,000, 35,000, 36,000, 37,000, 38,000, 39,000, 40,000, 41,000, 42,000, 43,000, 44,000 or 45,000. In various aspects, the number average molecular weight (Mn) can be between 1,000 to 2,000, 1,000 to 2,500, 1,000 to 3,000, 1,000 to 3,500, 2,000 to 2,500, 2,000 to 3,000, or 2,000 to 3,500.

In various aspects, n is greater than, or about, 15, 16, 17, 18, 19, 20, 21, 22, 23, 24, 25, 26, 27, 28, 29, 30, 31, 32, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109 or at least 110. In various aspects of the compound, n is less than 7, 8, 9, 10, 11, 12, 13, 14, 15, 16, 17, 18, 19, 20, 21, 22, 23, 24, 25. In various aspects of the compound, n is not one or more of 15, 16, 17, 18, 19, 20, 21, 22, 23, 24, 25, 26, 27, 28, 29, and 30.

In various aspects, the variable n can be chosen from any integer between 1 and 3, 1 and 5, 1 and 10, 2 and 10, 2 and 14, and between 20 and 110, for example n can be 5, 11, 60 or 106. For example, n can be any integer in the range of 2-15, 4-12, 5-11, 8-14, 9-14, 10-14, 11-14,8-12, 9-12, 10-12, 20-110, 30-110, 40-110, 50-110, 60-110, 20-106, 30-106, 40-106, 50-106, 60-106, 30-70, 40-70, 50-70, 55-65, 60-70, 30-80, 40-80, 50-80, 60-80, 70-120, 80-120, 90-120, 100-120, 70-110, 80-110, 90-110, or 100-110. In some aspects, the compound may include some strands having n in the range of 1-15, 4-12, 5-11, 8-14, 9-14, 10-14, 11-14, 8-12, 9-12 or 10-12 and also some strands having n in the range of 50-110, 60-110, 20-106, 30-106, 40-106, 50-106, 60-106, 30-70, 40-70, 50-70, 55-65, 60-70, 30-80, 40-80, 50-80, 60-80, 70-120, 80-120, 90-120, 100-120, 70-110, 80-110, 90-110, or 100-110.

In various aspects, the polydispersity index (PDI) is less than or about 1.25, 1.24, 1.23, 1.22, 1.21, 1.20, 1.19, 1.18, 1.17, 1.16, 1.15, 1.14, 1.13, 1.12, 1.11, 1.10, 1.09, 1.08, 1.07, 1.06, or less than or about 1.05. In various aspects, the PDI may be greater than 1.01, 1.02, 1.03, 1.04, 1.05, 1.06, 1.07, 1.08, 1.09, 1.10, 1.11, 1.12, 1.13, 1.14, 1.15, 1.16, 1.17, 1.18, or greater than 1.19.

### Antiviral Compositions and Antiviral-Treated Substrates.

The present disclosure provides an antiviral composition, or an antiviral substrate. The antiviral composition or substrate can include one or more compounds, such as any of the compounds described herein. The antiviral composition includes a carrier. The antiviral substrate includes a substrate.

In various aspects, the carrier can be a fluid. In various aspects, the compounds are dissolved in the carrier. In various aspects, the compounds are suspended in the carrier. In various aspects of the method, the carrier includes water. In various further aspects, the carrier includes one or more of an alcohol or organic solvent. In some aspects, the alcohol is ethanol. The carrier can further include detergents, solubilizers, chelators, a buffer system and colorants. The composition can be a solution or coating deposited on a wipe, a tissue, a bandage, a medical device, surgical instrument, tubing, a catheter, warfighter machinery, a sponge, a textile, a diaper, a counter-top, a food preparation surface, a wound dressing, a dressing for surgical incisions, a keyboard surface, a packing for wounds, a packing for surgical incisions, a nasal packing or a feminine care product, or a combination thereof. The composition can be a coating. In various aspects, the coating can be a transparent coating, a colorless coating, or a pigmented coating. For example, the coating can be a paint.

In various aspects, the carrier can be at a neutral pH, an acidic pH or a basic pH. For example, the carrier can have a pH of 6-8. In various aspects, the carrier has a pH greater than 8 or less than 6. In various aspects, the composition can be configured to deposit a residue of compounds on the treated object, area or surface.

The substrate can be a hard surface or a soft surface. In various aspects, the substrate is a fabric, a work surface, a medical device, packaging materials, personal protective equipment, a fibrous support, a wipe, a water filter, an air filter, a mask, or a combination thereof. In various further aspects, the substrate is a wipe, a tissue, a bandage, a medical device, surgical instrument, tubing, a catheter, warfighter machinery, a sponge, a textile, a diaper, a counter-top, a food preparation surface, a wound dressing, a dressing for surgical incisions, a keyboard surface, a packing for wounds, a packing for surgical incisions, a nasal packing or a feminine care product, or a combination thereof. The substrate can take many possible forms, including those described in U.S. Pat. Nos. 8,598,053, 8,618,009, 8,753,570, 9,005,540, 9,527,806, 9,750,250, 9,968,698, 10,092,000, 10,058,099, 10,174,042, 10,533,991, and PCT International Application Publication No. WO 2019/060586 A1, each of which is incorporated by reference herewith in their entirety.

In various aspects, the substrate can be configured to be reusable, for example, a reusable wipe, tissue, bandage, medical device, surgical instrument, tubing, catheter, warfighter machinery, sponge, textile, diaper, counter-top, food preparation surface, wound dressing, dressing for surgical incisions, keyboard surface, packing for wounds, packing for surgical incisions, nasal packing or feminine care product. Any such reusable substrate may have embedded antiviral compounds retained therein. Embedding compound in a substrate can be achieved by adjusting the length and size of polymeric compounds, or by providing the compound with functional groups that covalently or non-covalently bind the compound to the substrate. U.S. Pat. No. 9,750,250, which is incorporated by reference herewith in its entirety, describes adjusting chain length to embed, or permit easy deposit of, polyelectrolyte compounds.

In further aspects, the antiviral substrate can be any one or more of single use, disposable, and configured to function as a vehicle for providing disinfectant to an object, surface or area. For example, a single use, disposable and/or disinfectant-providing wipe, tissue, bandage, medical device, surgical instrument, tubing, catheter, warfighter machinery, sponge, textile, diaper, counter-top, food preparation surface, wound dressing, dressing for surgical incisions, keyboard surface, packing for wounds, packing for surgical incisions, nasal packing or feminine care product. Any such disposable substrate can, in various aspects, be configured to deposit a residue of compound. Such compound configured to be deposited can have, in various aspects, an n of less than 10, 11, 12, 13, 14, 15, 16, 17, 18, 19 or 20.

In various aspects of the substrate, about 1 μg/cm³ to about 10 μg/cm³ of the antiviral substrate is the compound. In some aspects, about 0.5 μg/cm³ to about 20 μg/cm³ or about 0.1 μg/cm³ to about 100 μg/cm³ of the antiviral substrate is the compound. In various aspects of the substrate, the compound is embedded in the substrate and remains at a concentration of about from about 0.1 μg/cm³ to about 10 μg/cm³ after washing the substrate. In various aspects of the substrate, the compound is deposited on a surface, object or area to be treated at a concentration of about from about 0.1 μg/cm³ to about 10 μg/cm³ after washing the substrate.

In various aspects of the substrate, the compound absorbs visible light. In various aspects, the compound can be configured to function as a passively photoactivated antimicrobial, e.g., by absorbing visible light to generate reactive oxygen species. Such antiviral substrates, in various aspects, can include compounds which primarily absorb light in the visible range.

In various aspects of the antiviral substrate, the compound absorbs UV light, e.g., UVA, UVB and/or UVC. For example, absorbs light in the range of 200-280 nm, 280-315 nm, and/or 315-380 nm. In various aspects, the compound can be configured to function as an actively photoactivated antimicrobial, e.g., by absorbing visible light to generate reactive oxygen species. Such antiviral substrates can, in various aspects, can include compounds which primary absorb light in the UV range, rather than in the visible range. Such compound can have, in various aspects, n is less than 10, 11, 12, 13, 14, 15, 16, 17, 18, 19 or 20. As a further example, the UV optimized compound may have a number average molecular weight (Mn) between 100 to 1,000, 500 to 1,000, 1,000 to 2,000, 1,000 to 2,500, 1,000 to 3,000, 1,000 to 3,500, 2,000 to 2,500, 2,000 to 3,000, or 2,000 to 3,500.

In various further aspects, the antiviral substrate may be configured to respond to the target light source by adjusting polymer size (e.g., the n value or number average molecule weight) and, also, by complexation with micelle forming agents and counterions (e.g., SDS and other detergents), by complexation with polysaccharides or by formulation as a hydrogel.

In various aspects of the antiviral substrate, the substrate is a polysaccharide. In various aspects of the antiviral substrate, the polysaccharide is alginate, carboxymethyl amylose or carboxymethyl cellulose. In various aspects, the antiviral substrate is in the form of a hydrogel. In some aspects, the antiviral substrate is in the form of an alginate hydrogel.

The compounds can be non-leachably bound to the substrate. In various aspects, the antiviral compound is leachably bound to the substrate. When the antiviral compound is non-leachably bound to the substrate, wiping a surface with the antiviral substrate can lead to substantially no transfer of the antiviral compound to the new surface. In some aspects, this transfer can be monitored by observing the fluorescence of the antiviral compound.

The compound can be in contact with at least one surface of the substrate. The compound can be substantially uniformly distributed on the substrate. One or more layers can separate the compound from the substrate.

The antiviral compound and substrate can exhibit antiviral properties, for example, the ability to destroy virus, inhibit plaque formation, or inhibit viral spread of infection.

The contacting can be performed in any suitable way. The contact can be performed by at least one of foamed applicators, cotton swabs, saturated swab sticks, saturated wipes, aerosols, sprays, brushes, and dips. In various aspects, the contacting is accomplished by at least on of an aerosol spray and spray. For example, the antiviral compound may be mixed with an aerosol propellant (e.g., dichlorodifluoromethane, trichlorofluoromethane, dichlorotetrafluoroethane, carbon dioxide or other suitable gas).

### Method of Inactivating SARS-CoV-2 Virus.

Various aspects provide a method of inactivating SARS-CoV-2 virus. The method includes contacting SARS-CoV-2 virus with a conjugated aromatic compound effective to inactivate the virus.

The method can optionally further include exposing the conjugated aromatic compound to light-irradiation, such as UV light, IR light, and/or visible light, while the conjugated oligomer or polymer is in contact with the SARS-CoV-2 virus. Exposing the conjugated aromatic compound to light-irradiation can be performed in any suitable way, such as by performing the contacting in conditions that include ambient lighting or enhanced lighting designed to increase the amount or rate of the inactivating effect of the conjugated aromatic compound on the SARS-CoV-2 virus. In some aspects, the compound is effective to inactivate SARS-CoV-2 virus in the dark.

An article or composition can include the conjugated aromatic compound, such as the antiviral-treated substrate described herein, such as a coating, paint, wipe, spray, mask, clothing, personal protective equipment, warfare fighter, or combination thereof. The method can include contacting a material including the conjugated aromatic compound with the SARS-CoC-2 virus. In various aspects, the present invention provides an article or composition for inactivating the SARS-CoC-2 virus, wherein the article or composition includes the conjugated aromatic compound.

The conjugated aromatic compound can include the structure:

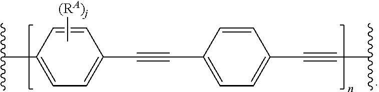

The conjugated aromatic compound can include the structure:

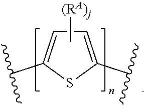

The conjugated aromatic compound can have the structure:

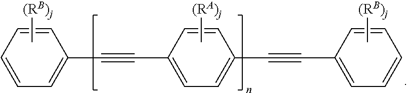

The variable n can be 1 to 10,000, such as 1 to 1,000, 1 to 100, 1 to 50, 1 to 10, 1 to 5, or less than 10,000 but equal to or greater than 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 500, 1,000, 1,500, 2,000, 2,500, 5,000, or 10,000 or less.

At each occurrence, j can be independently 1 or 2. The variable j can be 1. The variable j can be 2.

The variable R^{A} can be —H or R^{C}. The variable R^{B} can be independently —H or R^{C}.

The variable R^{C} can be —X—R¹—R². The variable X can be a bond, —O—, —NH—, or —S—. The variable R¹ can be substituted or unsubstituted (C₁-C₂₀)alkylene, such as (C₂-C₇)alkylene. The variable R² can be -(1,4-substituted 1,4-diazabicyclo[2.2.2]octane-1,4-diium)-R³, 3-R³-substituted imidazolium, pyridinium, —SO₃, —CO₂H, —CO₂⁻, —N⁺(R³)₃, —N⁺(R³)₂H, or —N(R³)₂. The variable R³ can be —H or substituted or unsubstituted (C₁-C₁₀)alkane. At least one R^{A} or R^{B} in the compound can be R^{C}, such that the compound includes at least one R^{C}. The variable R^{A} can be R^{C}. The variable R^{B} can be R^{C}.

The variable R² can be SO₃⁻. The variable R² can be -(1,4-substituted 1,4-diazabicyclo[2.2.2]octane-1,4-diium)-C₆H₁₃. The variable R² can be 3-methylimidazolium.

The variable R^{C} can be —O—(CH₂)₃—SO₃. The variable R^{C} can be —O—(CH₂)₃-3-methylimidazolium. The variable R^{C} can be —(CH₂)₇-3-methylimidazolium. The variable R^{C} can be —O—(CH₂)₂-(1,4-substituted 1,4-diazabicyclo[2.2.2]octane-1,4-diium)-C₆H₁₃.

The conjugated aromatic compound can include the structure:

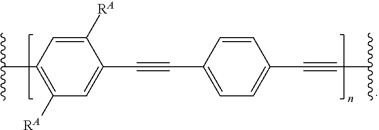

The conjugated aromatic compound can include the structure:

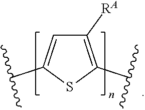

The conjugated aromatic compound can have the structure:

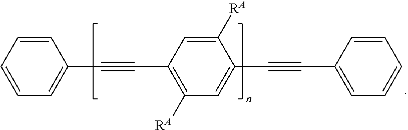

The conjugated aromatic compound can have the structure:

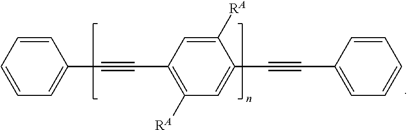

The conjugated aromatic compound can include one or more charge-balancing counterions. The charge-balancing counterion can be a halide, such as fluoride, chloride, iodide, or bromide. In other examples, the counterion can be nitrate, hydrogen sulfate, dihydrogen phosphate, bicarbonate, nitrite, perchlorate, iodate, chlorate, bromate, chlorite, hypochlorite, hypobromite, cyanide, amide, cyanate, hydroxide, permanganate. The counterion can be a conjugate base of any carboxylic acid, such as acetate or formate. In some aspects, a counterion can have a negative charge greater than −1, which can in some aspects complex to multiple ionized groups, such as oxide, sulfide, nitride, arsenate, phosphate, arsenite, hydrogen phosphate, sulfate, thiosulfate, sulfite, carbonate, chromate, dichromate, peroxide, or oxalate. The counterion can be ammonium (NH₄⁺), or an alkali metal such as sodium (Na⁺), potassium (K⁺), or lithium (Li⁺). In some aspects, the counterion can have a positive charge greater than +1, which can in some aspects complex to multiple ionized groups, such as Zn²⁺, Al³⁺, or alkaline earth metals such as Ca²⁺ or Mg²⁺.

The conjugated aromatic compound can have the structure:

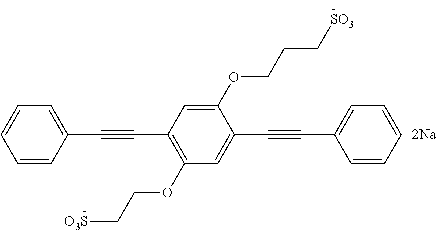

The conjugated aromatic compound can have the structure:

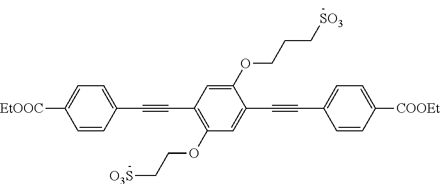

The conjugated aromatic compound can have the structure:

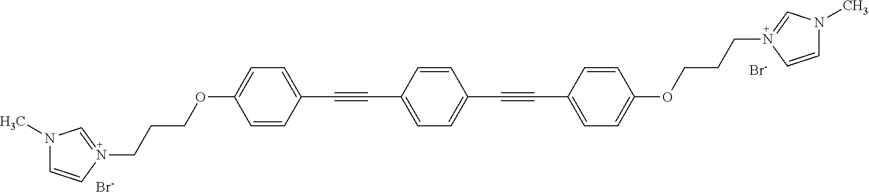

The conjugated aromatic compound can include the structure:

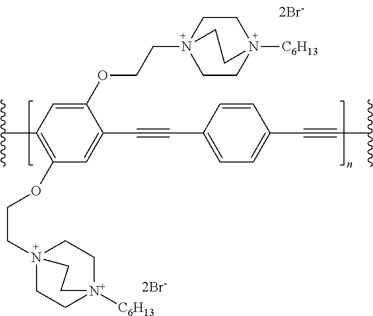

The conjugated aromatic compound can include the structure:

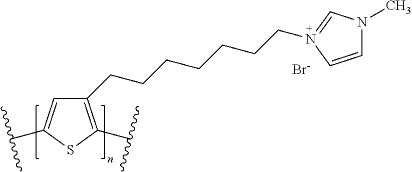

The conjugated aromatic compound can be Compound D:

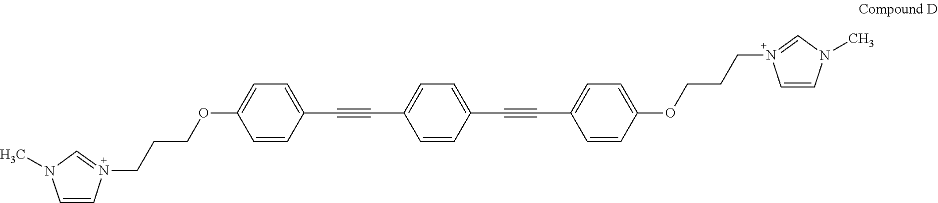

The conjugated aromatic compound can be Compound E:

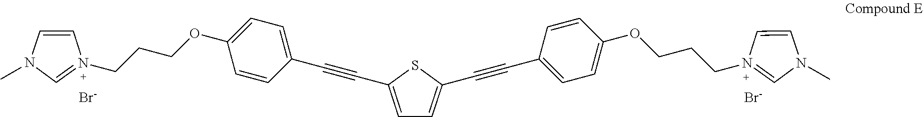

The conjugated aromatic compound can be Formula VII:

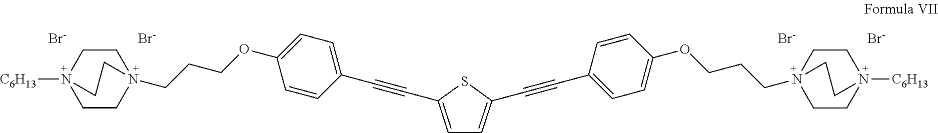

The conjugated aromatic compound can be Formula VIII:

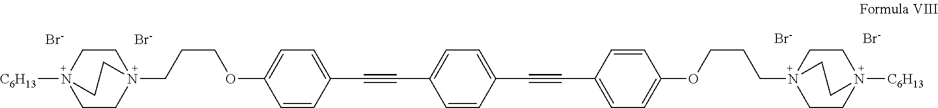

The conjugated aromatic compound can be Formula IX:

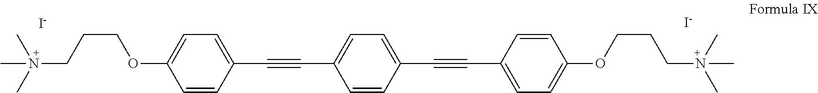

The conjugated aromatic compound can be Formula X:

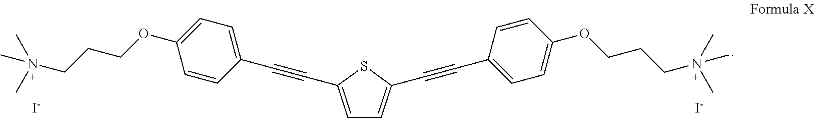

### EXAMPLES

Various aspects of the present invention can be better understood by reference to the following Examples which are offered by way of illustration. The present invention is not limited to the Examples given herein.

### Part I

### Materials and Methods.

Vero E6 cells (African Green Monkey Kidney Cells) were used as a cell culture for modeling SARS-CoV-2 infection and viability. Crystal violet solution was used for imaging plaques and staining healthy cells. Vero E6 cells are common cells used to define infectious particle concentration in viral stocks. These cells have an impaired type I IFN response and are therefore susceptible to virus infection and cell death.

Viral infectious activity and viability was evaluated by monitoring viral plaques. A viral plaque is formed when a virus infects a cell within the fixed cell monolayer. The virus infected cell will lyse and spread the infection to adjacent cells where the infection-to-lysis cycle is repeated. The infected cell area will create a plaque (an area of infected, dead cells surrounded by uninfected, live cells) which can be seen by adding a crystal violet solution to color the cytoplasm of healthy cells.

Four representative polyelectrolyte compounds were tested: Compound A, Compound B, Compound C, and Compound D. Compounds can be obtained according to synthetic routes described in U.S. Pat. Nos. 8,598,053, 8,618,009, 8,753,570, 9,005,540, 9,527,806, 9,750,250, 9,968,698, 10,092,000, 10,058,099, 10,174,042, 10,533,991, and PCT International Application Publication No. WO 2019/060586 A1, each of which is incorporated by reference herewith in their entirety.

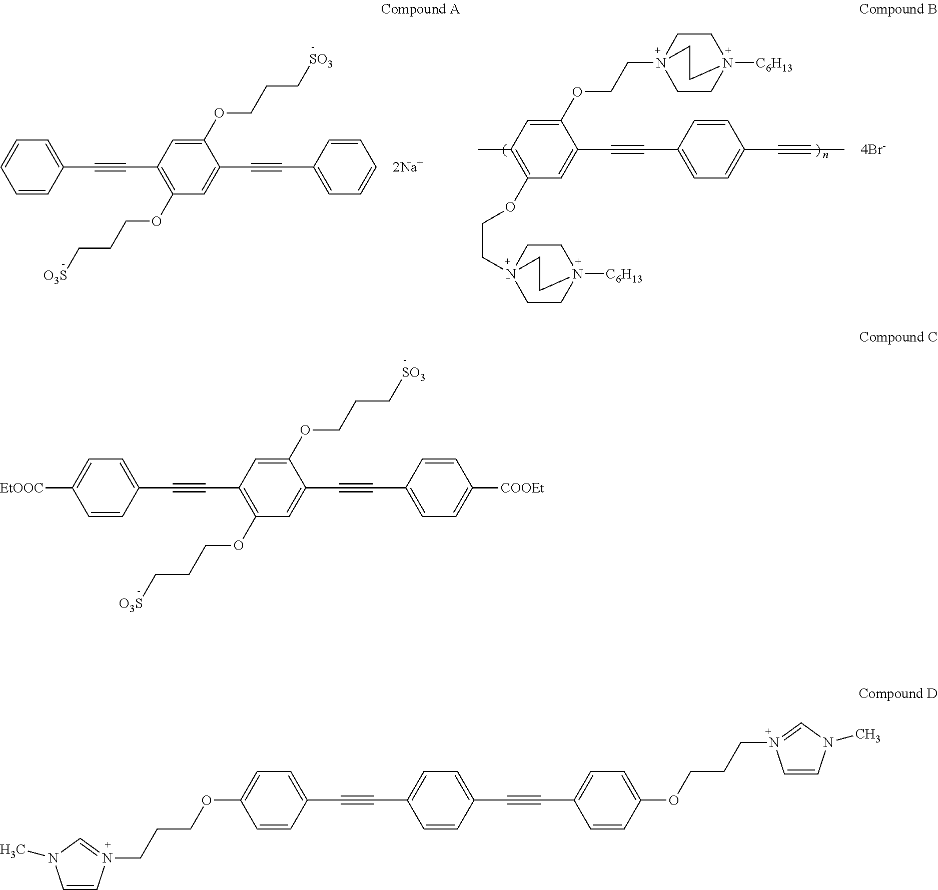

### Example I-1

A 10 μg/mL solution of each test compound A-D was mixed together with 2.5×10⁵ plaque forming units/mL of SARS-CoV-2 in a microtube under a photoreactor. Each mixture was subjected to pre-incubation by treating it with light at the 300 nM to 400 nM range for 10, 20, 30, or 60 minutes.

Each pre-incubated mixture was then transferred to a well of a multi-well assay plate containing a monolayer of Vero E6 cells and incubated for 1 hour at 37° C. to establish. The incubated mixture was then overlaid with a microcrystalline cellulose overlay medium (2.4% AVICEL®) and a high glucose medium supplemented with fetal bovine serum (2×DMEM/5% FBS).

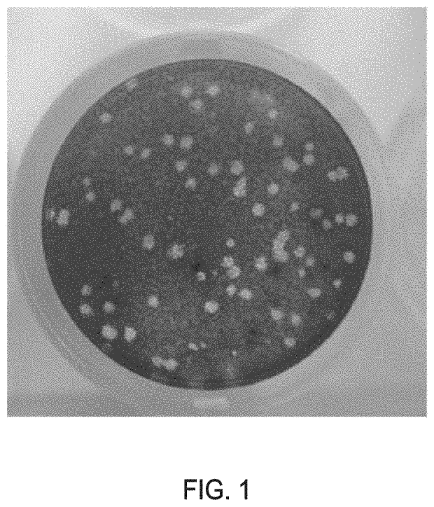

The cell culture was next incubated at 37° C. for 3 days and then stained. Each well was inspected and plaques were counted. FIG. 1 shows a photograph of a cell culture containing visible viral plaques.

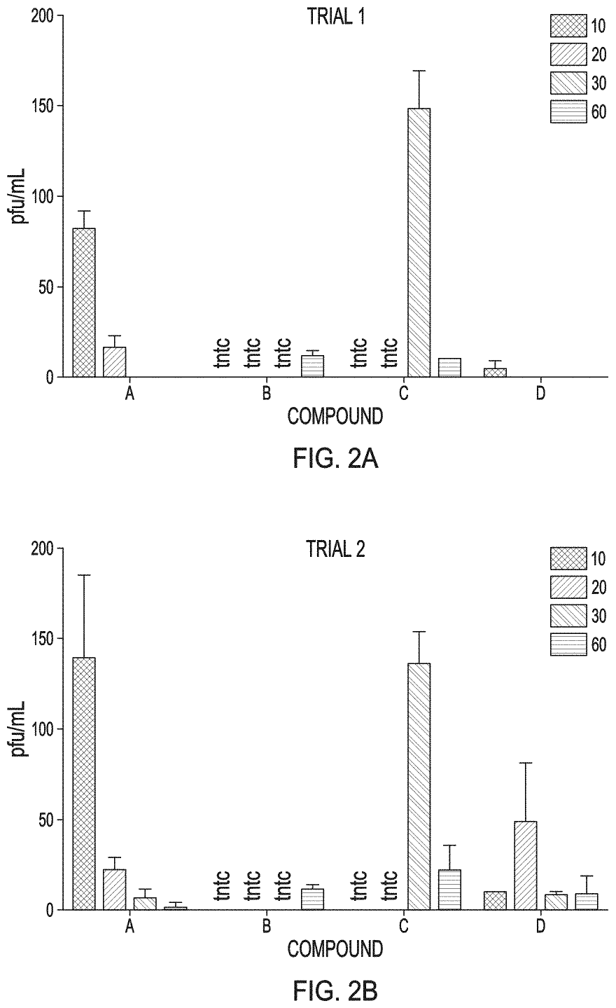

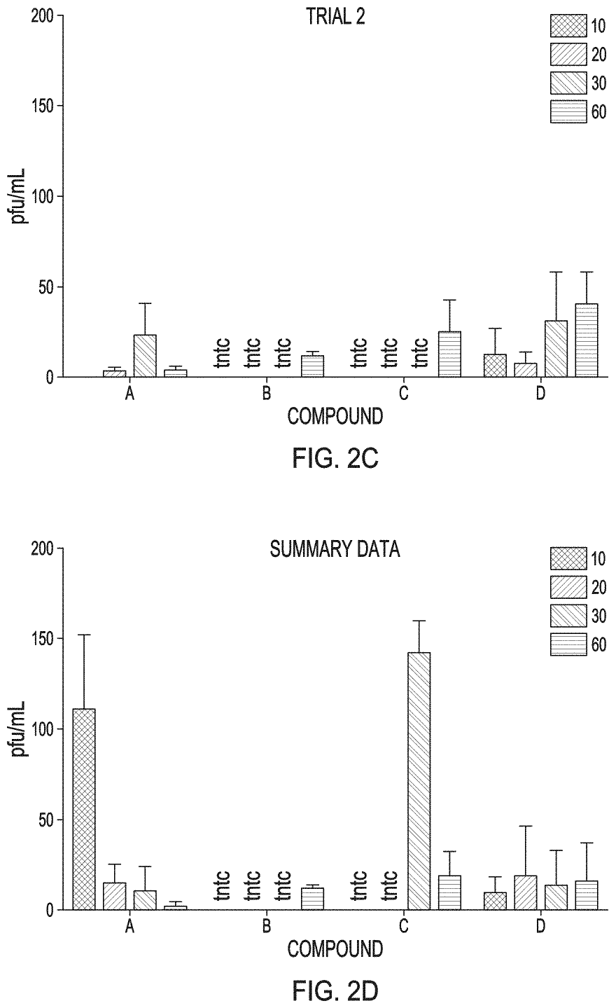

The compounds were tested at each of the various pre-incubation time periods in triplicate. FIGS. 2A-D shows graphs showing concentration of plaque forming units after treatment with Compound A, Compound B, Compound C, or Compound D, corresponding to trial 1 (FIG. 2A), trial 2 (FIG. 2B), trial 3 (FIG. 2C), as well as a summary graph (FIG. 2D). Each graph corresponds to an input concentration of 2×10⁵ pfu/mL, with measurable plaque reductions of 3-4 log for all samples (tntc=too numerous to count).

Samples containing polyelectrolyte compounds incubated in light demonstrated significant reduction in plaque number, depending on the antimicrobial used. Increased time of virus incubation with all compounds resulted in increased plaque reduction.

### Part I Comparative Examples

A comparative set of samples corresponding to those in Example I-1 was run with dark pre-incubation conditions. Specifically, samples having one of each of Compounds A-D (10 micrograms per mL), were incubated together with 2×10⁵ plaque forming units in the dark for 10, 20, 30 or 60 minutes.

Samples incubated in the dark conditions demonstrated no measurable reduction in viral plaques, independent of the antimicrobial used.

Another comparative sample set, containing just water was also prepared and incubated together with the virus particles in the presence of light (300-400 nm) for 10, 20, 30, or 60 minutes, but none resulted in any visible cytopathic effects.

Light treatment of virus incubated without a polyelectrolyte compound did not result in measurable differences in plaque forming units.

Lastly, a comparative set of samples corresponding to those in Example 1 was run without SARS-CoV-2 virus. Specifically, incubation of compounds diluted in cell culture media onto Vero E6 cell culture monolayers (in the absence of virus) did not result in any visible cytopathic effects.

### Results and Discussion.

All test compounds were shown to be effective at inhibiting the ability of SARS-CoV-2 to form plaques in the Vero E6 cell cultures. This inhibition of plaque formation indicates that polyelectrolytes have the ability to destroy, inactivate, or reduce the ability of coronavirus to spread.

The most effective compounds were two oligomer compounds, and included compounds having either cationic pendant groups or anionic pendant groups.

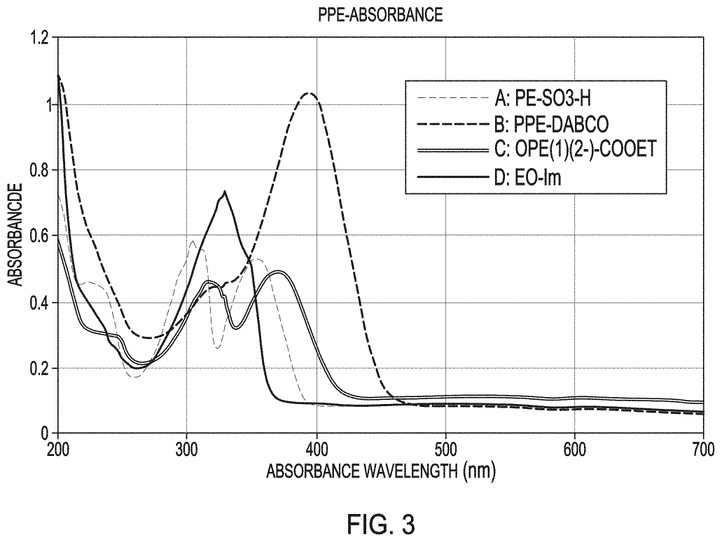

Of the tested compounds, one of the less effective compounds was a cationic polymer. The absorption spectra of each compound was further tested using a Lambda 35 U/Vis spectrometer (PerkinElmer, Waltham, Mass.) in quartz cuvettes (PerkinElmer, Waltham, Mass.), and reporting absorbance units. The solutions were made at 100 μg/mL in milli Q water. The absorption spectra, shown in FIG. 3, shows that the three oligomer polyelectrolytes absorb moderately in the range 300-400 while the polymer absorbs very strongly with a maximum peak absorbance around 380-400. Although the bioassay suggested that the polymer is the least effective among the four materials/compounds tested, it is possible that the high absorbance of the polymer provided an “inner filter affect” whereby the polymer absorbs too much light to effectively inactivate the virus.

Importantly, all tested polyelectrolytes were effective, regardless of whether pendant groups had a positive or negative charge. Without intending to be limited by theory, this result indicates that both positive- and negative-charged polyelectrolytes can engage in ionic or electrostatic interactions with the SARS-CoV-2 viral surface.

The result showing that the compounds did not achieve antiviral effects in the dark, (i.e., in the absence of visible or UV light) suggests that these compounds function different than conventional quaternary ammonium materials (or “Quats”), which do not have a have light-activated inhibition pathway. This result indicates that the presently described compositions and compounds in the presence of light can be effective against enveloped ssRNA, including SARS-CoV-2, even where conventional quaternary ammonium salts are ineffective. Moreover, it is expected that sunlight and/or room light, e.g., incandescent, fluorescent, or LED light, can serve as a “passive” catalysts for antiviral activity, based on the absorption spectra of various polyelectrolytes.

Prior to these results, it was not known if polyelectrolytes, whether positive or negative, would have surface interactions with SARS-CoV-2 or if they would have any inhibitory activity on the virus. Coronaviruses contain a viral envelope that protects them to a greater extent that other viruses such as bacteriophages, which can be more readily attacked by chemical agents. Studies demonstrated the ability of polyelectrolytes to inhibit various non-enveloped viruses, bacteria, fungus are not sufficient to provide any expectation that the same compounds and conditions would be effective against SARS-CoV-2 or other enveloped viruses. (See, e.g., U.S. Pat. No. 10,174,042, which is incorporated by reference herewith in its entirety). Efficacy against SARS-CoV-2 and other enveloped ssRNA could not be reasonably expected without the results of experimentation against such viruses. The surfaces of coronaviruses are complex, and it was unknown how polyelectrolytes would interact with the SARS-CoV-2 specifically. It was further unknown regarding what steps, such as preincubation, could be taken to advantageously inhibit the cytopathic effect SARS-CoV-2. Overall, the Examples of Part I provided herein demonstrate that polyelectrolyte compounds having either cationic or negative functional groups can provide unexpected advantages for antiviral applications, including for inhibiting cytopathic effects of SARS-CoV-2.

### Part II

### Example II-1

### Materials and Methods.

Cells and viruses. Vero E6 cells (ATCC, CRL-1586) were cultured in Dulbecco's modified Eagle's medium (DMEM) supplemented with 10% heat-inactivated FBS, 1% pen/strep, 2 mM L-glut, 1% non-essential amino acids, 1% HEPES. Severe acute respiratory syndrome coronavirus 2 (SARS-CoV-2, Isolate USA-WA1/2020) was acquired from BEI Resources (NR-52281) and propagated in Vero E6 cells for 3 days. Infectious virus was isolated by harvesting cellular supernatant and spinning at 1000 rpm for 10 minutes to remove cellular debris and stored at −80° C. SARS-CoV-2 infectious particles were quantified in media by standard plaque assay. Briefly, Vero E6 cells were treated with serial dilutions of SARS-CoV-2 in DMEM (4% FBS) and incubated at 37° C. for 1 hr. An overlay of 2.4% cellulose (colloidal microcrystalline, Sigma #435244) and 2×DMEM (5% FBS) was added to each well and incubated for 3 days at 37° C. and 5% CO₂. On day 3, cells were fixed with 4% paraformaldehyde and stained with crystal violet to visualize and count plaques.

Oligomer and polymer incubations with SARS-CoV-2. Individual compounds were diluted to 10 μg/mL or 5 μg/mL in DMEM culture media (4% FBS). SARS-CoV-2 was added to the media solution containing a compound at a final concentration of 2.6×10⁵ pfu/mL. Virus incubated in media was supplemented with a volume of RNA/DNAse-free water equal to that used for the compound dilutions. Samples were then incubated at room temperature inside a Luzchem light chamber and exposed to either no light, bulbs emitting 420 nm light, or bulbs emitting 300-400 nm light for the specified time intervals. The concentration of infectious virus present in the samples was then quantified by plaque assay.

### Introduction

It is hard to overstate the devastation brought to the world by the SARS-CoV-2 virus through the highly contagious Covid-19 disease. In contrast, of the six previously encountered human coronaviruses, four are common and remain active but pose only minor threats to world health. Three recent (since 2000) coronaviruses originally infected animals but have jumped to humans and pose existential threats to human health. SARS coronavirus (SARS-CoV) emerged in 2002 and caused Severe Acute Respiratory Syndrome (SARS). SARS-CoV emerged in an outbreak in February 2003 in Asia and rapidly spread to two dozen countries in Asia, South America, North America and Europe before being contained. According to the World Health Organization (WHO), there were 8,098 cases worldwide and of those 774 died. In the US there were 8 verified cases and no deaths and there have been no cases of SARS subsequent to 2003. Middle East Respiratory Syndrome (MERS) emerged in Saudi Arabia in 2012 and has since spread to several countries, including the US. MERS causes severe respiratory symptoms, including death. There have been two nonfatal confirmed cases in the US in May 2014. MERS is a threat to travelers to the Middle East and travel advisories are posted on the CDC Website. In contrast to the SARS and MERS, Covid-19 has spread to all inhabited parts of the world, excluding Antarctica. As of Sep. 5, 2020, there are 26,468,000 cases and 871,000 deaths worldwide, and of those 6,095,007 cases and 185,687 deaths have occurred in the US.

The major attempt at controlling the spread of Covid-19 includes several efforts to produce vaccines. Although promising candidates are already in human clinical trial, it is likely that unforeseen problems may delay the availability of a safe and effective vaccine and its widespread employment. Several diagnostic tests for of SARS-CoV-2 have been developed; however, uneven test distribution and delays in obtaining results have hindered our knowledge of viral spread and hampered out ability to develop an effective response. Several conventional disinfectants are active against the SARS-CoV-2 virus and the most commonly used of these are bleach, hydrogen peroxide, and alcohol solutions. While these disinfectants are effective for use as cleaning solutions and wipes, the volatility and/or corrosivity of the “active agents” limit prolonged sterilization of surfaces or objects by these reagents.

Here we present an alternative approach to long lasting disinfection: cationic phenylene ethynylene polymers and oligomers (conjugated electrolytes). Over the past decade we and others have demonstrated the effectiveness of these compounds and derivative materials against bacteria, fungi, biofilms, and viruses. The antimicrobial activity of these compounds includes both light-activated and dark-active pathways. Encouragingly, these materials exhibit relatively low toxicity against mammalian cells or the human skin. Depending upon the selection of materials and properties such as solubility and stability, it is possible to control the lifetime of these materials in coatings, with photodegradation as the main route of inactivation. The most common byproducts of photodegradation, due to oxidative cleavage, are aldehydes and carboxylic acids that are unlikely to damage the environment. In addition, these materials can also bind selectively with certain proteins or protein fragments and with light-irradiation, the conjugated electrolytes may photosensitize the oxidation of proteins at the specific binding sites.

The Examples of Part II report a pilot study of five phenylene ethynylene materials and compounds as inactivators of the SARS-CoV-2 virus in aqueous suspensions. The five materials were chosen as representatives of a much larger group of polymeric and oligomeric conjugated electrolytes that have been previously shown to be highly active against microbes. In this study, samples of each material in solution was incubated with an aqueous suspension of SARS-CoV-2. The samples were incubated in the dark and under UV-visible light irradiation in a photoreactor. After incubation for increasing amounts of time, the samples were analyzed for virus activity. As detailed below, it was found that all five of the tested materials were effective against the SARS-CoV-2 virus, but with effectiveness that differed significantly among the tested group.

### Results.

### Oligomer and Polymer Structures and Rationale for Selection.

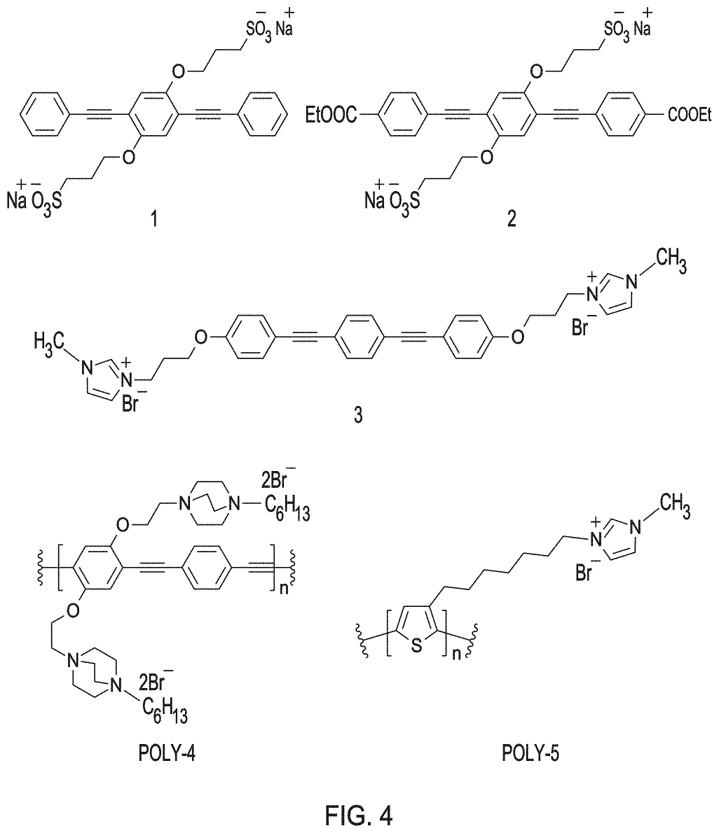

The structures of the five materials tested in Example II-1 are shown in FIG. 4. Three are oligomeric phenylene ethynylenes (OPEs, compounds 1, 2, and 3) and two are cationic conjugated polymers (poly-4 and poly-5).

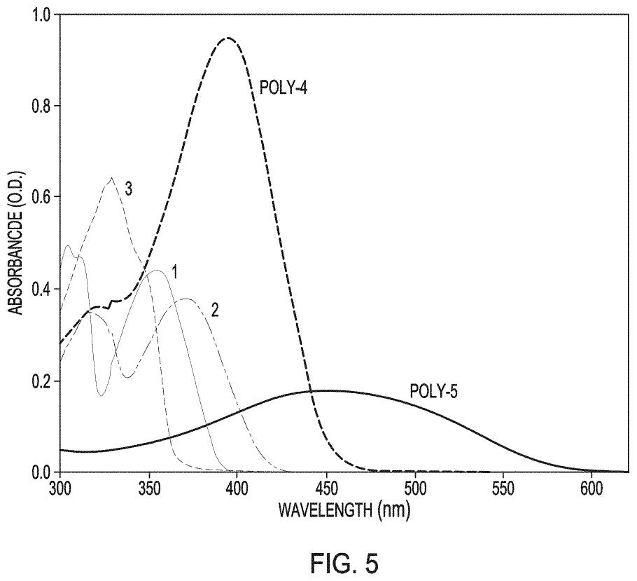

FIG. 5 illustrates absorption spectra of the five phenylene ethynylene materials at 10 μg/mL in water. Spectra were recorded on a Lambda 35 U/Vis spectrometer (PerkinElmer, Waltham, Mass.) in quartz cuvettes (PerkinElmer, Waltham, Mass.). As shown in FIG. 5, all of the materials absorb in the near-UV and/or visible regions of the spectrum. The final concentration of materials chosen for this study was 10 μg/mL in solutions that contain an active compound and SARS-CoV-2. This concentration was selected to give moderate absorption throughout the sample and yet minimizing a potential “inner filter” effect. The fact that the poly-4 absorbs much more strongly at this concentration suggests the possibility that an “inner filter” effect could diminish its light-activated viral inactivation activity.

Our previous studies testing the antiviral activities of oligomeric and polymeric phenylene ethynylene materials were performed with the non-enveloped RNA bacteriophages MS2 and T4. In contrast, SARS-CoV-2 is an enveloped virus containing several proteins that shield its internal RNA. Recent reports suggest that the virus has an acidic coat and might be reactive towards negatively charged reagents. We hypothesize that the viral envelope might have both positively and negatively charged regions that could interact with charged compounds such as OPEs and PPEs. Thus, the selection of the test compounds for the pilot project was in-part based on charge: oligomers 1 and 2 are negatively charged. Moreover, oligomer 2 has been shown to associate strongly with several proteins and in fact, induces protein oxidation upon irradiation. The cationic polymer poly-4 has become our workhorse compound for antimicrobial work. Cationic oligomer 3 has not been studied extensively but was selected for investigation due to our findings that phenylene ethynylene polymers, such as poly-5 containing pendant imidazolium groups are unusually reactive in light induced bacterial killing.

### Photoactive Oligomers Potently Inhibit SARS-CoV-2.

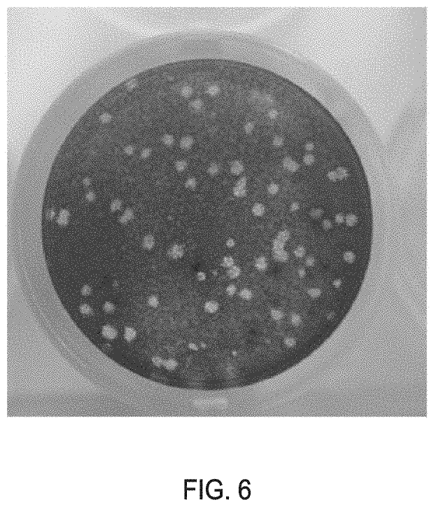

SARS-CoV-2 that causes Covid-19 efficiently infects and causes cytopathic effects in Vero E6 cells. Vero E6 cells have an impaired type I IFN response and are therefore highly susceptible to virus infection and cell death. Vero cells stained with crystal violet are commonly used to define infectious particle concentrations as expressed in plaque forming units (pfu) in viral stocks. A representative sample with a low pfu concentration is shown in FIG. 6. FIG. 6 illustrates a crystal violet stain of a Vero E6 cell monolayer 3 days post SARS-CoV-2 infection. A viral plaque, which appears as white circular features in the image, begins when a virus infects a cell within the cell monolayer. The virus infected cell subsequently lyses and spreads the infection to adjacent cells where the infection-to-lysis cycle is repeated. The infected cell area creates a plaque, an area of dead cells surrounded by uninfected, live cells, which can be seen by adding a crystal violet solution that colors the cytoplasm of healthy cells.

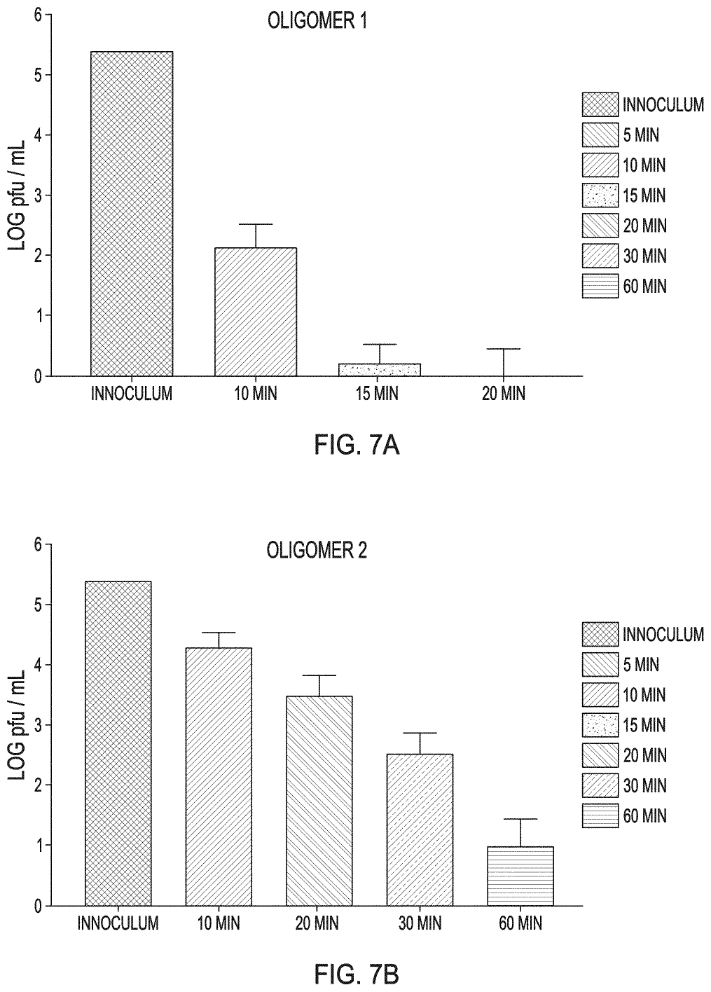

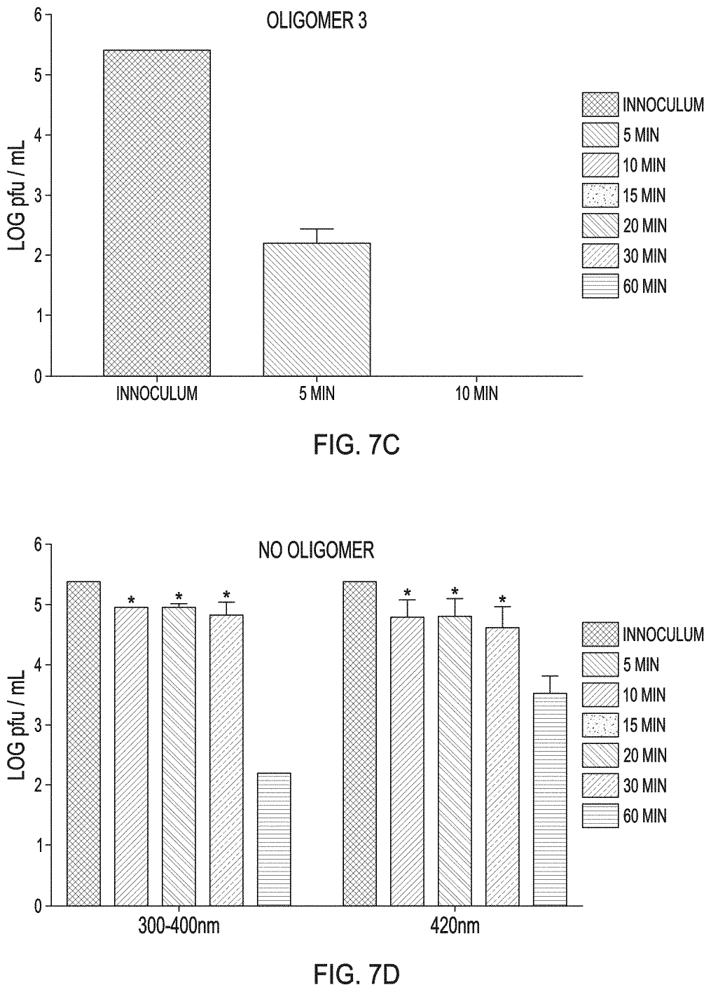

We first tested the near UV light (300-400 nm) activated inhibitory effect of oligomers 1, 2 and 3 against SARS-CoV-2 infection on Vero cells. FIGS. 7A-D illustrate antiviral activity of photoactive oligomers against SARS-CoV-2 with near UV light irradiation for the indicated time periods. 10 μg/mL of each oligomer was incubated with 2.6×10⁵ pfu/mL SARS-CoV-2 and irradiated. Viral titer was quantified by plaque assay on Vero E6 cells. Asterisks (*) denote that upper limit of detection was reached for plaque assays with these samples. Values shown are the average titer from at least three independent experiments (±SEM). FIGS. 7A-D shows the reduction in pfu when SARS-CoV-2 is mixed in cell culture media with the oligomers and incubated with near UV light irradiation. Compounds 1 and 3 demonstrated immediate activity and ablated any viral plaque formation as fast as 15-20 minutes of irradiation for oligomer 1 and 10 minutes irradiation for oligomer 3 in these conditions. While not as striking, oligomer 2 also showed a clear ability to decrease plaque formation by 30 minutes incubation and irradiation. Importantly, these oligomers demonstrated no antiviral activity when incubated in the absence of light and demonstrated no cytopathic effects when added to Vero cell monolayers in the absence of virus (data not shown).

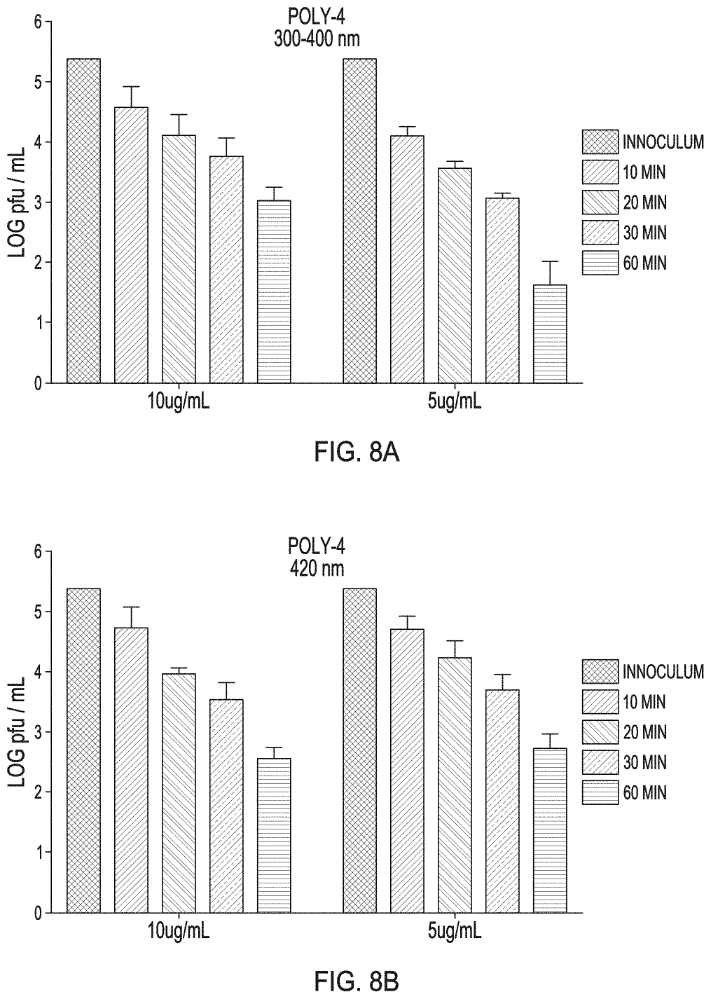

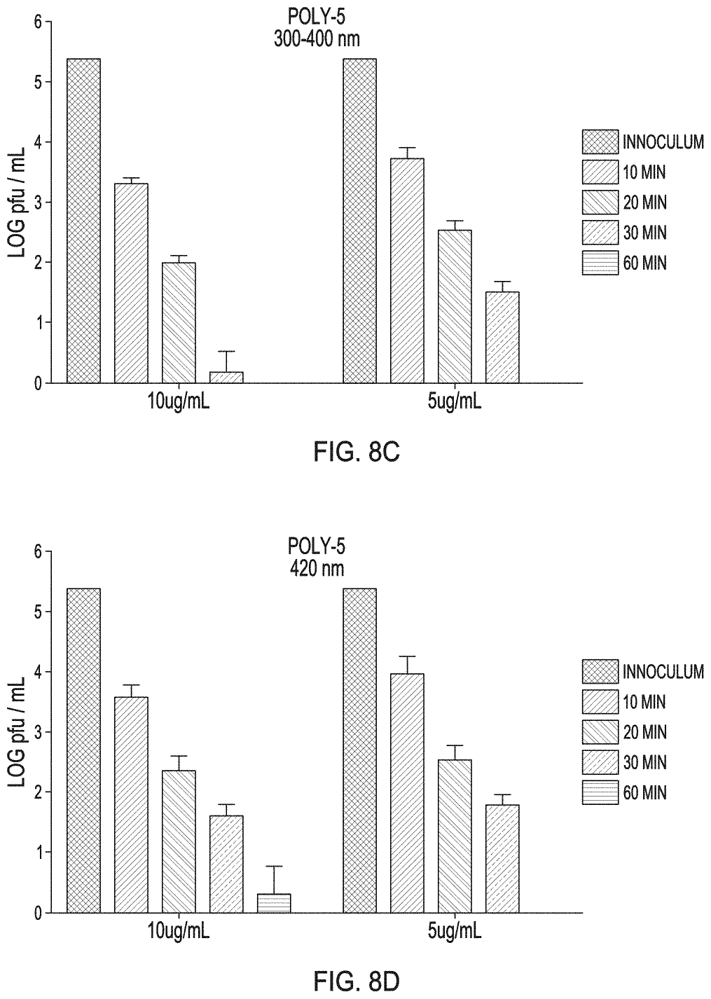

We then expanded our investigations to include photoactive polymers poly-4 and poly-5 which, unlike the oligomers, absorb in the visible region as well as the near-UV (FIG. 5), making them likely to be active at these wavelengths as well. FIGS. 8A-D illustrate antiviral activity of photoactive polymers against SARS-CoV-2. Poly-4 (A, B) or poly-5 (C, D) was incubated with 2.6×10¹ pfu/mL SARS-CoV-2 with near UV light or visible light irradiation for the noted times. Viral titer was quantified by plaque assay on Vero E6 cells. Values plotted are the average titer calculated from at least three independent experiments (±SEM). As shown in FIG. 8A, when incubated with SARS-CoV-2 at a concentration of 10 μg/mL, poly-4 reduced plaque formation by almost 1.5 logs after 20 minutes of exposure to near UV light, but this reduction is minimally greater than that of the control with no compound added when exposed to either near UV or visible light. In contrast, poly-5 is capable of effectively eliminating viral plaque formation in as little as 30 minutes incubation in near UV light and reducing viral titers by nearly 5 logs with 60 minutes of irradiation in the visible light (FIG. 8B). Because of the potential “inner filter” effect from poly-4 and poly-5 during light incubation, we tested their ability to inhibit SARS-CoV-2 plaque formation at a lower concentration of 5 μg/mL under the same conditions. This lower concentration of either polymer demonstrated minimal differences in viral titer compared to the inhibition observed using 10 μg/mL (FIG. 8C). Similar to the oligomer results, we observed neither cytopathic effects of the polymers alone on Vero cells nor reduction in viral titer by plaque assay when polymers were incubated with the virus in the dark (data not shown). We therefore conclude that the “inner filter” effect is not interfering with the antiviral activity of poy-4 and poly-5 in these conditions. Further, poly-5 demonstrated effective inhibition of SARS-CoV-2 in both near UV and visible ranges of light, making it a strong candidate for further study and potential use to combat viral environmental contamination.

### Discussion

The most important and significant result of this study is that all five materials tested show antiviral activity against SARS-CoV-2 under irradiation with light absorbed by the specific material. The three oligomers are active when irradiated with near UV light and the two polymers are active under both visible and near-UV irradiation. Both anionic (1 and 2) and cationic (3) oligomers are active; this is reasonable for non-covalent binding of the oligomers to protein components, likely the viral spike proteins, of the SARS-CoV-2. We have shown in a previous simulation study that the binding of phenylene ethynylene-based oligomers to the MS2 virus capsid protein assembly is mediated by strong van der Waals interactions between the hydrophobic OPE backbone and the capsid protein and strong electrostatic interactions between the OPE charged side chains and charged residues on the capsid surface. A combination of hydrophobic and electrostatic interactions between the oligomers and polymers and the SARS-CoV-2 spike proteins likely contribute to the compounds' antiviral activities in this study. Of the 4 structural protein components, the membrane protein M is a likely target for both oligomers and polymers to bind and be in close proximity to interior RNA. As shown in FIG. 7D, when suspensions of SARS-CoV-2 are irradiated with near UV or visible light without oligomers or polymers present, there is some inactivation of the virus, especially upon prolonged irradiation with near UV. However, it is clear that most of the inactivation of the virus under irradiation is due to excitation of the oligomers and polymers.

None of the five materials studied thus far exhibit antiviral activity in the dark. This is not surprising since SARS-CoV-2 is an enveloped virus and in the dark the interactions between oligomers and polymers and membrane proteins are not expected to be strong enough to denature the protein or break covalent bonds. However, it seems to be the case that binding brings the oligomers and polymers sufficiently close to the virus such that the reactive oxygen species (ROS) generated at the binding sites on the virus can penetrate the virus and cause damages to the membrane coat protein M, RNA and/or inner proteins. Quite likely, the smaller oligomers may penetrate into the interior of the virus perhaps even binding to the N-protein that is directly connected to the virus RNA. Since we have shown in other studies that both negatively and positively charged OPEs can bind with misfolded proteins or protein fragments, binding of the polymers and oligomers used in this study with virial proteins and subsequent generation of singlet oxygen by irradiation close to the RNA seems a likely path for virus inactivation. The observation that the cationic quaternary ammonium poly-4 is inactive in the dark is a harbinger that classic quaternary ammonium surfactants may not be particularly active against the virus.

As mentioned previously and detailed in FIGS. 7A-D, anionic oligomer 1 and cationic oligomer 3 show rapid and effective inactivation of SARS-CoV-2 under irradiation with near UV light with essentially complete inactivation in 10-15 min, while anionic oligomer 2 shows much slower inactivation under the same conditions. While oligomer 2 has been shown in other studies to bind strongly to misfolded proteins and fragments, it seems reasonable as 2 is more hydrophilic than 1 or 3 and better solubilized in water but less able to penetrate into the interior of the virus. The curious effect that oligomer 2 is less active than 1 may also be associated with the fact that the ester groups have been shown to give rise to rapid excited state quenching of the oligomer in water. This quenching may reduce the ability of the compound to sensitize reactive oxygen species.

For the two polymers used in this study, irradiation with either near UV or visible light results in inactivation of the SARS-CoV-2 virus as shown in FIGS. 8A-D. Poly-5 shows rapid and efficient inhibition of the virus with essentially complete inactivation in 20-30 min under either near UV or visible irradiation at levels of both 5 and 10 μg/mL. Poly-4 is significantly less reactive than Poly-5. At this point it is premature to speculate on the cause of the differences. An attractive feature of both polymeric systems examined is that they are reactive under visible irradiation and potentially can be “passive” antiviral agents under ambient irradiation conditions.

It is worth considering the practical applications of these materials. We can envision that these materials can be used in the prevention of Covid-19 and other virus-based diseases as well as for future virus threats. In previous studies we have shown that similar materials prepared in our laboratories can be incorporated into surface coatings. For example, textiles where compound are covalently attached via electrospinning or non-covalently incorporated by adsorption have exhibited broad spectrum antimicrobial properties. Other studies have shown that these materials are not likely to harm the environment from their degradation byproducts and also not harmful to human skin or to several other types of mammalian cells. It seems likely that these materials, used as pure components or mixtures in wipes, sprays, personal protective equipment (PPE) items, clothing for athletes, warfare fighters, and paints and coatings, can provide lasting disinfection of hard surfaces in rooms, vehicles, outdoor and indoor spaces.

### Conclusions

In this pilot study, we have tested five representative conjugated oligomers and polymers from an array of phenylene ethynylene-based cationic and anionic conjugated materials against SARS-CoV-2, the virus that causes the Coved-19 disease. All five of the materials investigated show moderate to very strong inactivation of the virus on irradiation with near-UV or visible light. Although the oligomers and polymers are active under irradiation, they do not inactivate the virus in the dark. The antiviral activities of the compounds are likely due to binding of the compounds to viral proteins that brings the compounds in close proximity to the virus, followed by light-activated singlet oxygen and ROS generation that ultimately damage and inactivate the virus.

Our results show that we can obtain highly effective light induced inactivation with several of these oligomers and polymers including irradiation with near-UV and visible light. With both the oligomers and polymers, we can reach several logs of inactivation with relatively short irradiation times. Our results suggest several applications involving the incorporation of these materials in wipes, sprays, masks and clothing and other Personal Protection Equipment (PPE) that can be useful in preventing infections and the spreading of this deadly virus and future outbreaks from similar viruses. Our findings open the way towards a host of applications that may mitigate disease infections from SARS-CoV-2 as well as current and future virus threats to human health.

### Part III

### Example III-1

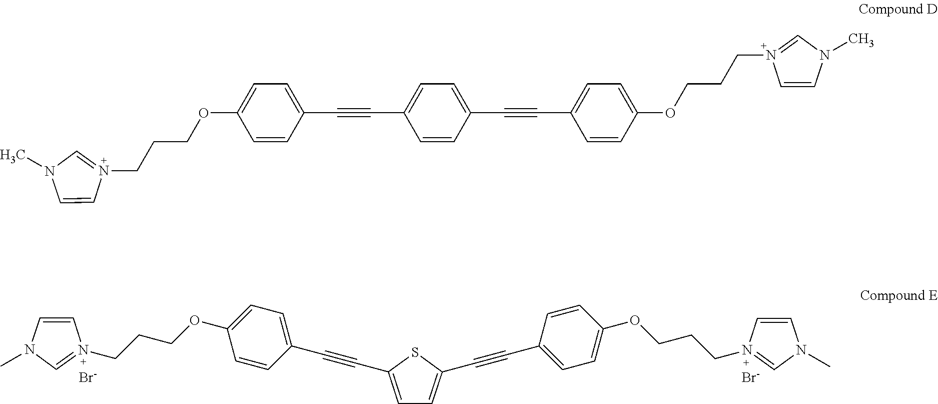

Compounds D and E were both observed to have excellent light-activated killing of bacteria (E coli). No killing of bacteria in the dark has yet been observed. Compound D was more reactive than Compound E, but both showed excellent anti-bacterial activity. Both compounds showed excellent anti-bacterial activity using LuzChem “cool white” light.

Compound E was applied to a wet wipe surface and also to the surface of a filter for filtration of air or water. Compound E on these surfaces showed strong light-activated killing of E. coli, and activity was retained when the surface was immersed in a suspension of E. coli.

Compound E showed no toxicity against mammal models.

“Cool white” consumer bulbs are good mimics for artificial light by LuzChem “cool white” light. This suggests that a “passive” activity under indoor lights may occur without applying a strong light source, with likely slowed rate of killing.

The good results on the wet wipe and air/water filter are evidence that the compounds herein may be useful for the coating of wipes, masks, filters for air or water, or for sprays or other coating applications.

### Part IV

### Example IV-1

### Materials and Methods.

Preparation of filters. A stock solution of conjugated cationic oligomer (100 μg/mL) was prepared by dissolving the compound in water. A piece of glass fiber filter (3×6 cm², 127 mg) was cut and immersed into 50 mL of conjugated cationic oligomer solution for 12 hrs. After soaking, the filter was removed from solution and dried at room temperature for 3 days. In order to calculate how much compound was absorbed by the glass fiber filter, UV-Vis spectra of the solution were taken before and after immersing and removing the filter. 0.84 mg of coated glass fiber filter was cut to use for each sample for a final oligomer concentration of 10 μg/mL in the plaque assay as described below to determine antiviral activity.

Cells and viruses. Vero E6 cells (ATCC, CRL-1586) were cultured in Dulbecco's modified Eagle's medium (DMEM) supplemented with 10% heat-inactivated FBS, 1% pen/strep, 2 mM L-glut, 1% non-essential amino acids, and 1% HEPES. Severe acute respiratory syndrome coronavirus 2 (SARS-CoV-2, Isolate USA-WA1/2020) was acquired from BEI Resources (NR-52281) and propagated in Vero E6 cells for 3 days. Infectious virus was isolated by harvesting cellular supernatant and spinning at 1000 rpm for 10 minutes to remove cellular debris and stored at −80° C. SARS-CoV-2 infectious particles were quantified in media by standard plaque assay. Briefly, Vero E6 cells were treated with serial dilutions of SARS-CoV-2 in DMEM (4% FBS) and incubated at 37° C. for 1 hr. An overlay of 2.4% cellulose (colloidal microcrystalline, Sigma #435244) and 2×DMEM, 5% FBS was added to each well and incubated for 3 days at 37° C., 5% CO₂. On day 3, cells were fixed with 4% paraformaldehyde and stained with crystal violet to visualize and count the number of plaque forming units (pfu).

The following compounds are discussed in this Example, along with the compounds shown in FIG. 4:

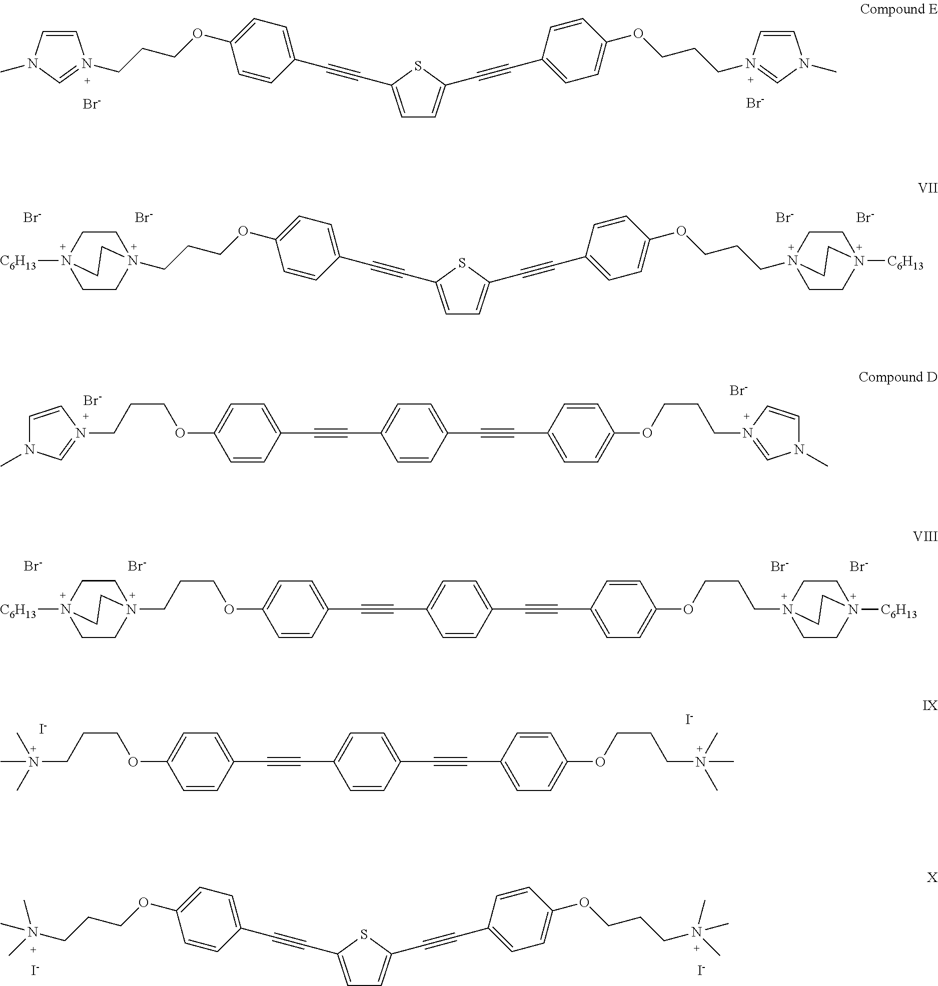

### Compound E Inactivation of SARS-CoV-2.

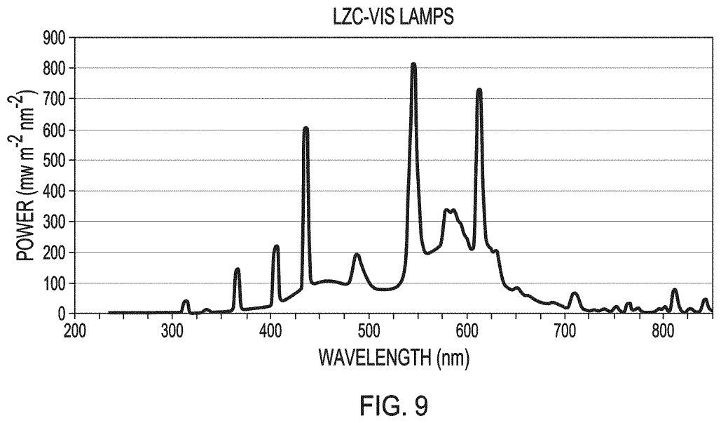

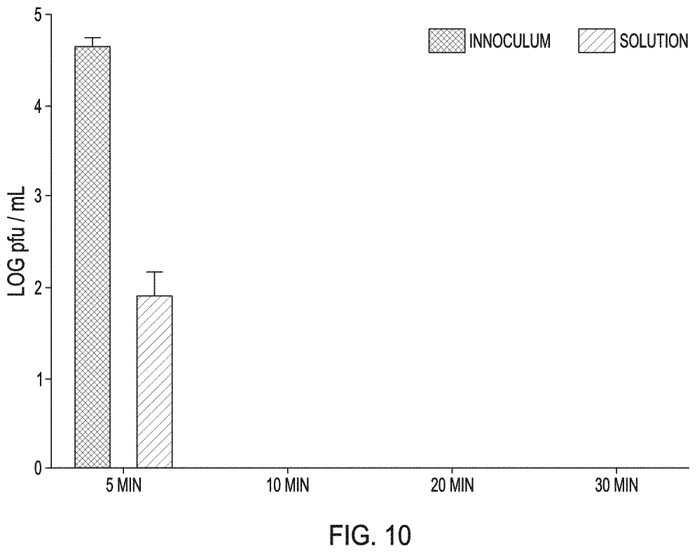

A solution of Compound E was diluted to 10 μg/mL in DMEM culture media (4% FBS). SARS-CoV-2 was added to Compound E: media solution at a final concentration of 1×10⁵ pfu/mL. Virus incubated in media supplemented with a volume of RNA/DNAse-free water equal to that used for the compound dilutions served as a negative control. Samples were then incubated at room temperature inside a Luzchem light chamber (using an LZC-Vis lamp; see FIG. 9 below for the lamp's emission spectrum) and exposed to the “cool white” light for the specified time intervals. Infectious virus present in the samples was then quantified by plaque assay. FIG. 10 illustrates antiviral activity of 10 μg/mL of Compound E in solution against SARS-CoV-2. Viral titer was quantified by plaque assay on Vero E6 cells. Values shown are the average titer from at least three independent experiments (±SEM). Inoculum is a control sample containing untreated virus and was not irradiated.

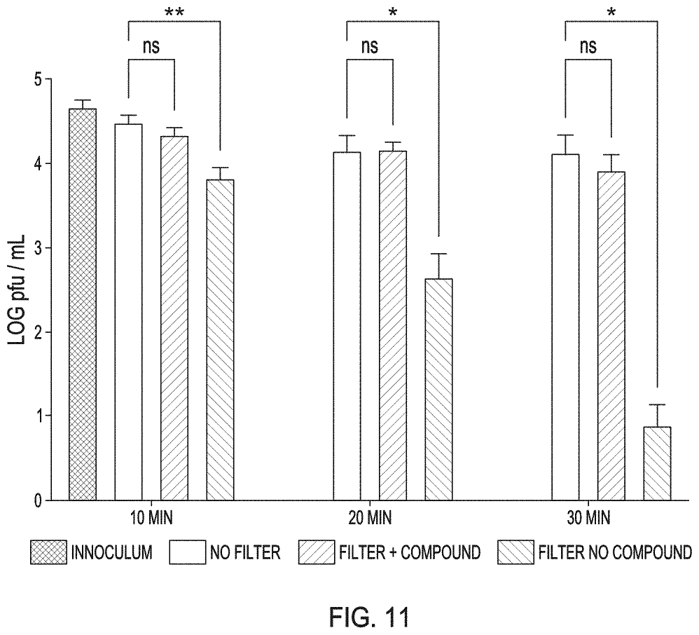

For experiments testing Compound E-coated filter inactivation, a media solution of SARS-CoV-2 at a concentration of 1×10⁵ pfu/mL was added to 1.5 mL tubes containing a Compound E-coated filter at a solution concentration of 10 μg/mL and gently agitated by pipetting up and down multiple times. As a negative control, filters not coated with Compound E were incubated with viral preparations and treated similarly. Samples were then incubated at room temperature inside a Luzchem light chamber and exposed to “cool white” light for the specified time intervals. Infectious virus present in the samples was then quantified by plaque assay. FIG. 11 illustrates antiviral activity of glass fiber filter coated with Compound E against SARS-CoV-2. 1×10⁵ pfu/mL SARS-CoV-2 was incubated with filter coated with Compound E (filter+compound), uncoated filter material (filter no compound), or no filter in the presence of “cool white” light for the indicated times. Inoculum is a control sample containing untreated virus and sample was not irradiated. The amount of Compound E present in the “filter+compound” samples is equivalent to 10 μg/mL in the sample solution. Viral titer was quantified by plaque assay on Vero E6 cells. Values shown are the average titer from at least three independent experiments (±SEM). * denotes p<0.05, two-way ANOVA with multiple comparisons correction.

### Discussion

In our prior work, the antiviral activities of oligomeric and polymeric phenylene ethynylene materials (OPEs and PPEs) were evaluated with the non-enveloped RNA bacteriophages MS2 and T4. The SARS-CoV-2 virus is an enveloped virus containing several proteins that shield its internal RNA. Recent reports suggest that the virus has an acidic coat and might be reactive towards negatively charged reagents. We hypothesized that the viral envelope might have both positively and negatively charged regions that could interact with charged compounds such as OPEs and PPEs. Thus, the selection of the test compounds shown in FIG. 4 for earlier work was in-part based on charge: oligomers 1 and 2 are negatively charged. Moreover, oligomer 2 has been shown to associate strongly with several proteins and in fact, induces protein oxidation upon irradiation. The cationic polymer poly-4 has become our workhorse compound for recent antimicrobial work. Cationic oligomer 3 has not been studied extensively but was selected for investigation due to our findings that phenylene ethynylene polymers, such as poly-5 containing pendant imidazolium groups are unusually reactive in light induced bacterial killing. Subsequent studies involved irradiation with “cool white” lamps that mimic office lighting in an interior, artificial light illuminated environment. Thus, materials irradiated with “cool white” and/or 420 nm light may show passive light-activation without the need for an intense light source, albeit perhaps at a slower rate.

Our pilot study with “cool white” lamps included poly-5 and an oligomer having the structure=formula VI. Both of these materials produced rapid inactivation of SARS-CoV-2 on irradiation with “cool white” lamps in a photoreactor (FIG. 9). Poly 5 is polythiophene P3HT-Im with 50-60 repeat units and showed inactivation during the first hour of irradiation with no residual virus at 2 or more hours. Formula X showed no virus present at 30 minutes irradiation, the earliest point in this study. These findings indicated that visible light-absorbing cationic oligomers might be ideal candidates for additional antiviral investigations. Towards these ends we investigated the synthesis, properties and antiviral activity of Compounds D, E, and Formulas VII-X. Our specific interest in these compounds was developed by analyzing components of the oligomers studied in our pilot study. Compounds D, E, and Formulas VII-X all represent the smallest member of a real or projected group of polymeric materials that may consist of “n” polymer repeat units. As we have shown earlier, these compounds are not truly conjugated due to structural variations generated by torsional rotation about the phenyl-acetylene group; instead we have an absorption spectrum that consists of additive “segment chromophores” that provide intense absorption in the near-UV and visible spectral regions. Thus, if we compare structures VII and X we see that both cationic oligomers have positively charged groups that offer the possibility of enhancing binding to the envelope of a cell or virus by Coulombic attraction to negatively charged regions of the pathogen envelope. However, while the two cationic groups of VII have a positive charge that is delocalized through the imidazole ring and hence probably more diffuse than the quaternary ammonium groups of Formula X. Another difference is in the conjugated portion of the structures of VII and IX. For VII the aromatic structure has a thiophene as the central ring which provides photoinduced electronic excitation of the chromophore by visible light and a heavy atom induced rapid intersystem crossing of the excitation from an excited singlet state to a triplet and hence efficient excitation of ambient molecular oxygen to excited singlet oxygen. For IX the positive charges on the quaternary ammonium group provide stronger bonding to anionic regions of the pathogen (bacterial or viral) and potential dark antimicrobial activity. However, in contrast to VII, oligomer IX is not active under visible light irradiation but is active against bacteria in the dark.

In the current study we have observed Compound E, Compound D, and Formula X in aqueous media with suspensions of E. coli and SARS-CoV-2 for Compound E and with suspensions of E. coli for Compound D and Formula X. We have also studied these oligomers as supported coatings on various supporting materials including wipe scaffolds, commercial Kimwipes, and proprietary materials from Diamond Wipes and HYDAC.

Both Compound E and Compound D are very active against E. coli suspended in solution under irradiation with visible light for Compound E and near UV for Compound D. In each case solutions containing 1-10 μg/mL show significant killing (˜99%) after five minutes of irradiation and complete killing at longer times (no survival indicated). However, neither compound showed any killing under dark incubation. This contrasts with the behavior of other compounds such as Formula X which have quaternary ammonium charged groups and show considerable dark reactivity, as well as activity under visible light and near-UV.

While Formula X was not studied with SARS-Cov-2, Compound E was irradiated in solution with suspensions of SARS-CoV-2 and exhibited >95% inactivation of the virus in 5 minutes of irradiation with “cool white” irradiation and complete deactivation inactivation after 10 minutes or longer irradiation.

The oligomers could be coated onto a variety of paper and fabric-like scaffolds to assess their activity in filtration or wipe applications. For Compound E coated on a scaffold of glass fiber material, light activation (“cool white”) resulted in about 90% inactivation after 10 minutes irradiation and 3 log inactivation after 30 minutes. With this experimental design, the coated sample was quite small (0.84 mg of coated glass fiber filter) and there was no filtration but the coated glass fiber filter was stirred by pipetting the solution up and down at the beginning of the irradiated incubation. The current tests serve as a proof of principal of useful employment of Compound E as a potent antiviral reagent for filtration of water and/or air.

Compound E had activity against E. coli in suspension and against E. coli and SARS-Cov-2 in suspension and on a filter, in visible light and near-UV, but not in dark conditions. Compound D had activity against E. coli in suspension under near-UV, but not under visible light or dark conditions. Formula X had activity against E. coli in suspension under visible light, near-UV, and dark conditions.

The terms and expressions that have been employed are used as terms of description and not of limitation, and there is no intention in the use of such terms and expressions of excluding any equivalents of the features shown and described or portions thereof, but it is recognized that various modifications are possible within the scope of the aspects of the present invention. Thus, it should be understood that although the present invention has been specifically disclosed by specific aspects and optional features, modification and variation of the concepts herein disclosed may be resorted to by those of ordinary skill in the art, and that such modifications and variations are considered to be within the scope of aspects of the present invention.

### EXEMPLARY ASPECTS

The following exemplary Aspects are provided, the numbering of which is not to be construed as designating levels of importance:

Aspect 1 provides a method of inhibiting an enveloped ssRNA virus, comprising contacting the virus with a composition comprising a compound having the structure of Formula I or Formula II in the presence of light,

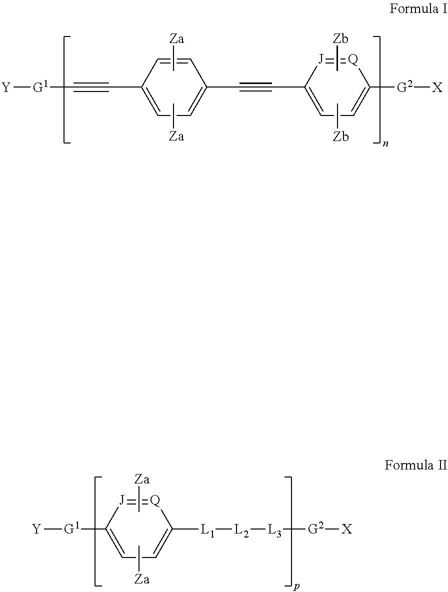

wherein

each of X and Y is independently H, COOR, O—(CH₂)ₘ-T, NH₂, or COR;

each of Za and Zb is independently H, O—(CH₂)ₘ-T, O—C₂H₄—(OCH₂)ₘ—R;

each of G¹ and G² is independently a bond, C₂C₆H₄, C₆H₄, C₂C₄S, or C₄S;

J and Q are each C or CH so as to provide a benzene ring, or J and Q are together S so as to provide a thiophene ring;

n is 1 to 200;

p is 1 to 10,000;

m is 0 to 10;

each of R is independently methyl, ethyl, n-propyl, isopropyl, phenyl, t-butyl, isobutyl, n-butyl, pentyl, hexyl, heptyl, octyl, cyclopropyl, cyclobutyl, cyclopentyl, cyclyhexyl, methoxymethyl, or trifluoromethyl;

each of T is independently H, SO₃⁻, COO⁻, COOR, DABCO, N-alkyl DABCO, imidazolyl, N-alkyl imidazolyl, NR₂, NHR₂⁺, or NR⁺;

L1 is independently a bond or —C≡C—;

L2 is independently a bond, a substituted or unsubstituted phenylene, thiophenylene, azulenylene, heptalenylene, biphenylene, indacenylene, fluorenylene, phenanthrenylene, triphenylenylene, pyrenylene, naphthacenylene, chrysenylene, biphenylenylene, anthracenylene, and naphthylene;

L3 is independently a bond or —C≡C—; and

at least one occurrence of Y, X, Za, and Zb is independently O—(CH₂)ₘ-T.

Aspect 2 provides the method of Aspect 1, wherein the enveloped ssRNA virus is a coronavirus.

Aspect 3 provides the method of any one of Aspects 1-2, wherein the enveloped ssRNA virus is SARS-CoV-2.

Aspect 4 provides the method of any one of Aspects 1-3, wherein the contacting is for at least 60 minutes.

Aspect 5 provides the method of any one of Aspects 1-4, wherein the contacting is in the presence of visible light or UV light.

Aspect 6 provides the method of any one of Aspects 1-5, wherein the composition is an aqueous solution or suspension.

Aspect 7 provides the method of any one of Aspects 1-6, wherein the compound is present in the composition at a concentration of about 1 μg/mL to about 100 mg/mL.

Aspect 8 provides the method of any one of Aspects 1-7, wherein the compound has a maximum absorbance between 250 nm and 700 nm at less than 380 nm.

Aspect 9 provides the method of any one of Aspects 1-8, wherein the compound has a maximum absorbance between 250 nm and 700 nm of less than 0.8 A.

Aspect 10 provides the method of any one of Aspects 1-9, wherein the compound has a molecular weight of less than 2000 Da.

Aspect 11 provides the method of any one of Aspects 1-10, wherein the compound has the structure of formula I and n=1.

Aspect 12 provides the method of any one of Aspects 1-11, wherein

each of X and Y is independently H or COOR;

each of Za is O—(CH₂)ₘ-T;

G¹ is C₆H₄;

G² is a bond;

J and Q are each CH so as to provide a benzene ring;

n is 1;

p is 1 to 10,000;

m is 2 to 3;

each of R is methyl or ethyl;

each of T is SO₃, N-hexyl DABCO, or N-methyl imidazolyl;

L1 is —C≡C—;

L2 is unsubstituted phenylene; and

L3 is —C≡C—.

Aspect 13 provides the method of any one of Aspects 1-11, wherein the compound has the structure of Formula I and wherein:

each of X and Y is O—(CH₂)ₘ-T;

each of Za is H;

G¹ is C₆H₄;

G² is a bond;

J and Q are each CH so as to provide a benzene ring;

n is 1;

m is 3;

each of R is independently methyl or ethyl; and

each of T is independently N-alkyl imidazolyl.

Aspect 14 provides the method of any one of Aspects 1-11, wherein the compound has the structure of Formula I and wherein:

each of X and Y is independently H or COOR;

each of Za is O—(CH₂)ₘ-T;

G¹ is C₆H₄;

G² is a bond;

J and Q are each CH so as to provide a benzene ring;

n is 1;

m is 3;

each of R is ethyl; and

each of T is SO₃⁻.

Aspect 15 provides a method of reducing SARS-CoV-2 viability on a substrate, comprising:

treating a substrate with a composition comprising a compound having the structure of Formula I or Formula II and exposing the substrate to SARS-CoV-2 in the presence of light,

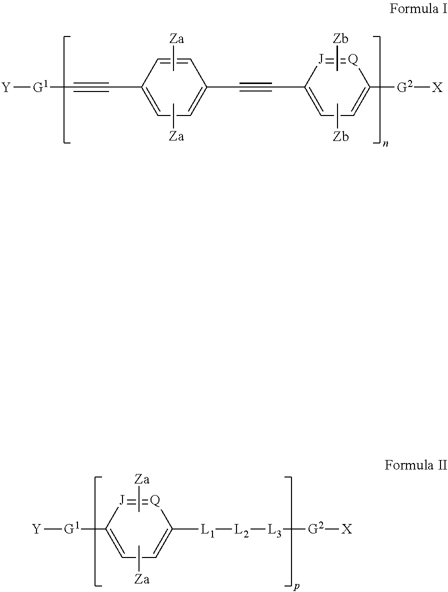

wherein

each of X and Y is independently H, COOR, O—(CH₂)ₘ-T, NH₂, or COR;

each of Za and Zb is independently H, O—(CH₂)ₘ-T, O—C₂H₄—(OCH₂)ₘ—R;

each of G¹ and G² is independently a bond, C₂C₆H₄. C₆H₄, C₂C₄S, or C₄S;

J and Q are each C or CH so as to provide a benzene ring, or J and Q are together S so as to provide a thiophene ring;

n is 1 to 200;

p is 1 to 10,000;

m is 0 to 10;

each of R is independently methyl, ethyl, n-propyl, isopropyl, phenyl, t-butyl, isobutyl, n-butyl, pentyl, hexyl, heptyl, octyl, cyclopropyl, cyclobutyl, cyclopentyl, cyclyhexyl, methoxymethyl, or trifluoromethyl;

each of T is independently H, SO₃⁻, COO⁻, COOR, DABCO, N-alkyl DABCO, imidazolyl, N-alkyl imidazolyl, NR₂, NHR₂⁺, or NR₃⁺;

L1 is independently a bond or —C≡C—;

L2 is independently a bond, a substituted or unsubstituted phenylene, thiophenylene, azulenylene, heptalenylene, biphenylene, indacenylene, fluorenylene, phenanthrenylene, triphenylenylene, pyrenylene, naphthacenylene, chrysenylene, biphenylenylene, anthracenylene, and naphthylene;

L3 is independently a bond or —C≡C—; and

at least one occurrence of Y, X, Za, and Zb is independently O—(CH₂)ₘ-T.

Aspect 16 provides the method of Aspect 15, further comprising incubating the treated and exposed substrate at about 35° C. or greater for at least 60 minutes in the presence of light.

Aspect 17 provides the method of any one of Aspects 15-16, wherein the substrate is in contact with SARS-CoV-2, or expected to come in contact with SARS-CoV-2.

Aspect 18 provides the method of any one of Aspects 15-17, wherein the substrate is a fabric, a work surface, a medical device, packaging materials, personal protective equipment, a water filter, a fibrous support, a wipe, an air filter, a mask, or a combination thereof.

Aspect 19 provides the method of any one of Aspects 15-17, wherein the substrate is personal protective equipment.

Aspect 20 provides the method of Aspect 19, wherein the personal protective equipment is a gown, scrub, glove, hair cover, face mask, sleeve, cuff, respirator, face shield, glasses, goggles, or apron.

Aspect 21 provides the method of any one of Aspects 15-20, wherein the substrate is a wipe, a tissue, a bandage, a medical device, a surgical instrument, a sponge, a textile, a diaper, a counter-top, a food preparation surface, a wound dressing, a dressing for surgical incisions, a keyboard surface, a packing for wounds, a packing for surgical incisions, a nasal packing, and a feminine care product.

Aspect 22 provides a method of reducing surface transmission of SARS-CoV-2 on a surface, comprising:

treating a substrate surface with a composition comprising a compound having the structure of Formula I or Formula II

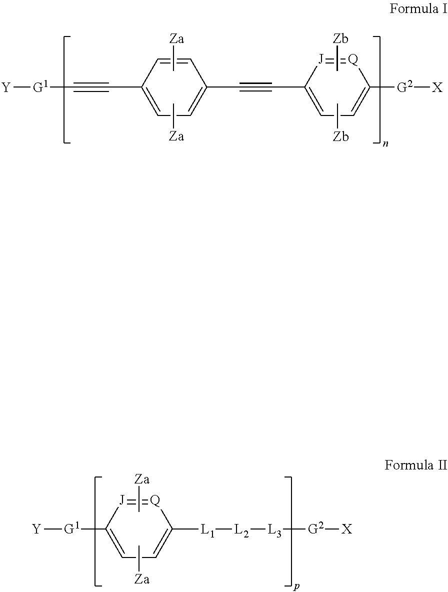

wherein

each of X and Y is independently H, COOR, O—(CH₂)ₘ-T, NH₂, or COR;

each of Za and Zb is independently H, O—(CH₂)ₘ-T, O—C₂H₄—(OCH₂)ₘ—R;

each of G¹ and G² is independently a bond, C₂C₆H₄. C₆H₄, C₂C₄S, or C₄S;

J and Q are each C or CH so as to provide a benzene ring, or J and Q are together S so as to provide a thiophene ring;

n is 1 to 200;

p is 1 to 10,000;

m is 0 to 10;

each of R is independently methyl, ethyl, n-propyl, isopropyl, phenyl, t-butyl, isobutyl, n-butyl, pentyl, hexyl, heptyl, octyl, cyclopropyl, cyclobutyl, cyclopentyl, cyclyhexyl, methoxymethyl, or trifluoromethyl;

each of T is independently H, SO₃⁻, COO⁻, COOR, DABCO, N-alkyl DABCO, imidazolyl, N-alkyl imidazolyl, NR₂, NHR₂⁺, or NR₃⁺;

L1 is independently a bond or —C≡C—;

L2 is independently a bond, a substituted or unsubstituted phenylene, thiophenylene, azulenylene, heptalenylene, biphenylene, indacenylene, fluorenylene, phenanthrenylene, triphenylenylene, pyrenylene, naphthacenylene, chrysenylene, biphenylenylene, anthracenylene, and naphthylene;

L3 is independently a bond or —C≡C—; and

at least one occurrence of Y, X, Za, and Zb is independently O—(CH₂)ₘ-T; exposing the substrate to SARS-CoV-2; and

incubating the treated and exposed substrate for at least 60 minutes in the presence of light.

Aspect 23 provides the method of Aspect 22, wherein the treated and exposed substrate is incubated at about 35° C. or greater wet.

Aspect 24 provides a method of inhibiting SARS-CoV-2 in circulated air and/or in water, comprising:

contacting a filter with a composition comprising a compound having the structure of Formula I or Formula II

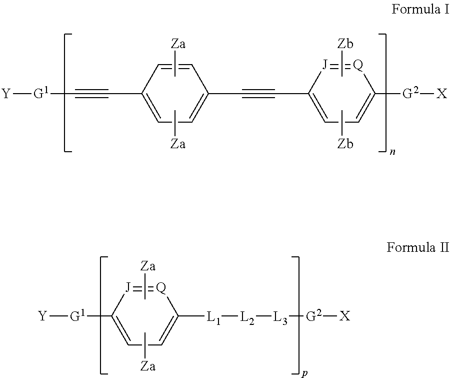

wherein

each of X and Y is independently H, COOR, O—(CH₂)ₘ-T, NH₂, or COR;

each of Za and Zb is independently H, O(CH₂)ₘ-T, O—C₂H₄(OCH₂)ₘ—R;

each of G¹ and G² is independently a bond, C₂C₆H₄, C₆H₄, C₂C₄S, or C₄S;

J and Q are each C or CH so as to provide a benzene ring, or J and Q are together S so as to provide a thiophene ring;

n is 1 to 200;

p is 1 to 10,000;

m is 0 to 10;

each of R is independently methyl, ethyl, n-propyl, isopropyl, phenyl, t-butyl, isobutyl, n-butyl, pentyl, hexyl, heptyl, octyl, cyclopropyl, cyclobutyl, cyclopentyl, cyclyhexyl, methoxymethyl, or trifluoromethyl;

each of T is independently H, SO₃⁻, COO⁻, COOR, DABCO, N-alkyl DABCO, imidazolyl, N-alkyl imidazolyl, NR₂, NHR₂⁺, or NR₃⁺;

L1 is independently a bond or —C≡C—;

L2 is independently a bond, a substituted or unsubstituted phenylene, thiophenylene, azulenylene, heptalenylene, biphenylene, indacenylene, fluorenylene, phenanthrenylene, triphenylenylene, pyrenylene, naphthacenylene, chrysenylene, biphenylenylene, anthracenylene, and naphthylene;

L3 is independently a bond or —C≡C—; and

at least one occurrence of Y, X, Za, and Zb is independently O—(CH₂)ₘ-T; exposing the filter to SARS-CoV-2 from circulated air;

optionally heating the filter to temperature of 35° C., optionally in the presence of light; and

circulating the air and/or the water through the filter, such as for a period of at least 60 minutes.

Aspect 25 provides the method of Aspect 24, wherein the filter has a pore size of about 0.1 micron or less.

Aspect 26 provides the method of any one of Aspects 1-10 and 15-24, wherein the compound has the structure of Formula III

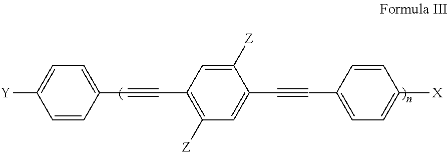

wherein

X is COOEt, Y is H, Z is OCH₂CH₂CH₂N(CH₃)₃⁺, and n is selected from the group consisting of numbers between 1 and 10; or

X is Y and is selected from the group consisting of H, CO₂Et, COO⁻, NH₂ and COCH₃, Z is OCH₂CH₂CH₂N(CH₃)₃⁺, and n is selected from the group consisting of numbers between 1 and 10; or

X is Y═OCH₂CH₂CH₂N(CH₃)₃⁺, Z is H, and n is 1.

Aspect 27 provides the method of any one of Aspects 1-10 and 15-24, wherein the compound has the structure of Formula IV

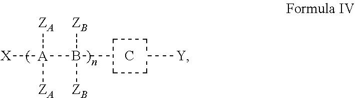

wherein

the poly(phenylene ethynylene) is grafted to the material by chemisorption or wherein the poly(phenylene ethynylene) is attached to the material by physisorption;

n is selected from the group consisting of whole numbers between 5 and 200;

A is C₂C₆H₂;

B is selected from the group consisting of C₂C₆H₂ and C₂C₄S;

C is either C₆H₄ or not present;

X is selected from the group consisting of H, [C₂C₆H₄]₂COOCH₂CH₃, and [C₂C₄H₂S][C₂C₆H₄]COOCH₂CH₃;

Y is selected from the group consisting of H and COOCH₂CH₃;

Z_{A} is selected from the group consisting of O(CH₂)ₖ(C₆H₁₂N₂)C₆H₁₃²⁺, O(CH₂)ₖSO₃⁻, O(CH₂)ₖN(CH₂CH₃)₃⁺, and O(CH₂)ₖN(CH₃)₃⁺, wherein k is selected from the group consisting of the whole numbers between 1 and 10;

Z_{B} is selected from the group consisting of H and (OCH₂CH₂)₃OCH₃;

wherein, it ZA is O(CH₂)ₖ(C₆H₁₂N₂)C₆H₁₃²⁺, then Z_{B} is H, A=C₂C₆H₂, C, if present, is C₆H₄, and X is selected from the group consisting of H and [C₂C₆H₄]₂COOCH₂CH₃;

if Y is H, then X is not H;

if X is H, then Y is not H, and C is not present;

if X is [C₂C₆H₄]₂COOCH₂CH₃, then Y is COOCH₂CH₃ and C is C₆H₄;

if Z_{A} is O(CH₂)ₖSO₃⁻, then Z_{B} is H, A=C₂C₆H₂, C is not present, and one but not both of X or Y is H;

if Z_{A} is O(CH₂)ₖN(CH₂CH₃)₃⁺, then Z_{B} is (OCH₂CH₂)₃OCH₃, A=C₂C₆H₂, C is not present, and one but not both of X or Y is H;

if Z_{A} is O(CH₂)ₖN(CH₃)₃⁺, and Z_{B} is (OCH₂CH₂)₃OCH₃, then C is not present, A=C₂C₆H₂ and one but not both of X or Y is H;

if Z_{A} is O(CH₂)ₖN(CH₃)₃⁺, Z_{B} is H, and C is not present, then A=C₂C₄H₂S and one but not both of X or Y is H;

if Z_{A} is O(CH₂)ₖN(CH₃)₃⁺, Z_{B} is H, and C is present, then Y═COOCH₂CH₃ and X is selected from the group consisting of [C₂C₆H₄]₂COOCH₂CH₃ and [C₂C₄H₂S][C₂C₆H₄]COOCH₂CH₃, and

if X is [C₂C₆H₄]₂COOCH₂CH₃, then B═C₂C₆H₂,

if X is [C₂C₄H₂S][C₂C₆H₄]COOCH₂CH₃, then B═C₂C₄S.

Aspect 28 provides the method of any one of Aspects 1-10 and 15-24, wherein the compound has the structure of Formula IV

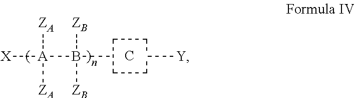

wherein

the poly(phenylene ethynylene) is grafted to the material by chemisorption or wherein the polymer is attached to the material by physisorption;

n is selected from the group consisting of whole numbers between 5 and 200;

A is C₂C₆H₂;

B is selected from the group consisting of C₂C₆H₂ and C₂C₄S;

C is either CH₄ or not present;

X is selected from the group consisting of [C₂C₆H₄]₂COOCH₂CH₃ and [C₂C₄H₂S][C₂C₆H₄]COOCH₂CH₃;

Y is COOCH₂CH₃;

Z_{A} is selected from the group consisting of O(CH₂)ₖ(C₆H₁₂N₂)C₆H₁₃²⁺, O(CH₂)ₖSO₃⁻, and O(CH₂)<N(CH₃)₃⁺; where k is selected from the group consisting of the whole number between 1 and 10;

Z_{B} is H;

if Z_{A} is O(CH₂)ₖ(C₆H₁₂N₂)C₆H₁₃²⁺, then A=C₂C₆H₂, C is C₆H₄, Y is COOCH₂CH₃, and X is [C₂C₆H₄]₂COOCH₂CH₃; and

if Z_{A} is O(CH₂)ₖN(CH₃)₃⁺ and C is present, then Y═COOCH₂CH₃ and X is selected from the group consisting of [C₂C₆H₄]₂COOCH₂CH₃ and [C₂C₄H₂S][C₂C₆H₄]COOCH₂CH₃,

if B═C₂C₆H₂, then X is [C₂C₆H₄]₂COOCH₂CH₃, and

if B═C₂C₄S, then X is [C₂C₄H₂S][C₂C₆H₄]COOCH₂CH₃.

Aspect 29 provides the method of any one of Aspects 1-10 and 15-24, wherein the compound has the structure of Formula IV

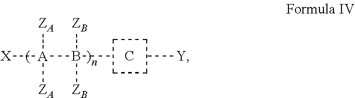

wherein

n is selected from the group consisting of 1, 2, 3 and 4;

A is selected from the group consisting of C₂C₆H₂ and C₂C₄S;

B═C₂C₆H₂;

C is either C₆H₄ or not present;

X is selected from the group consisting of COOCH₂CH₃, O(CH₂)ₖN(CH₃)₃⁺, O(CH₂)ₖSO₃⁻, and O(CH₂)ₖ(C₆H₁₂N₂)C₆H₁₃;

Y is selected from the group consisting of COOCH₂CH₃, O(CH₂)ₖN(CH₃)₃⁺, O(CH₂)ₖSO₃⁻, C₆H₂(OCH₃)₃, and O(CH₂)ₖ(C₆H₁₂N₂)C₆H₁₃²⁺;

k is selected from the group of whole numbers from 1 to 10;

Z_{A} is selected from the group consisting of H and O(CH₂)ⱼ(C₆H₁₂N₂)C₆H₁₃²⁺; where j is selected from the group of whole numbers from 1 to 10; and

Z_{B}═H;

wherein:
if X═COOCH₂CH₃ and Y═COOCH₂CH₃, then A=B═C₂C₆H₂, C is C₆H₄, and Z_{A} is O(CH₂)ₖ(C₅H₁₂N₂)C₆H₁₃²⁺;
if Z_{A} is H, A is C₂C₆H₂, X═O(CH₂)ₖN(CH₃)₃⁺, and C═C₆₋₁₄, then Y is selected from the group consisting of COOCH₂CH₃, O(CH₂)ₖSO₃⁻, C₆H₂(OCH₃)₃, and O(CH₂)ₖ(C₆H₁₂N₂)C₆H₁₃²⁺;
if Z_{A} is H, A is C₂C₆H₂, X═O(CH₂)ₖN(CH₃)₃⁺, C═C₆H₄, and B═C₂C₆H₂, then k is selected from the group consisting of 1, 2, 4, 5, 6, 7, 8, 9, and 10;
if Z_{A} is H, A is C₂C₆H₂, X═O(CH₂)ₖN(CH₃)₃⁺, and C is not present, then Y═C₆H₂(OCH₃)₃;
if Z_{A} is H, A is C₂C₆H₂ and X═O(CH₂)ₖSO₃⁻, then C═C₆H₄ and Y═O(CH₂)ₖSO₃⁻;
if Z_{A} is H, A is C₂C₆H₂, and X═O(CH₂)ₖ(C₅H₁₂N₂)C₆H₁₃²⁺, then C═C₆H₄ and Y═O(CH₂)ₖ(C₆H₁₂N₂)C₆H₁₃²⁺;
if A is C₂C₄S, then C═C₆H₄ and X is selected from the group consisting of O(CH₂)ₖN(CH₃)₃⁺ and O(CH₂)ₖ(C₆H₁₂N₂)C₆H₁₃²⁺; and
if X is O(CH₂)ₖ(C₆H₁₂N₂)C₆H₁₃²⁺, then Y═O(CH₂)ₖ(C₆H₁₂N₂)C₆H₁₃⁺.

Aspect 30 provides the method of any one of Aspects 1-10 and 15-24, wherein the compound has the structure of Formula V

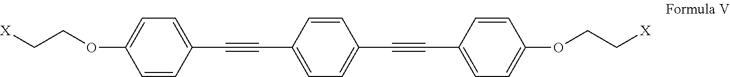

wherein X is n-hexyl DABCO, n-methyl imidazolium, or trimethylammonium.

Aspect 31 provides the method of any one of Aspects 1-10 and 15-24, wherein the compound has the structure:

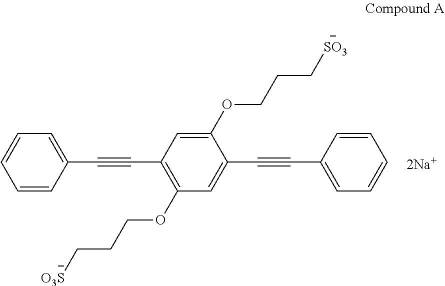

Aspect 32 provides the method of any one of Aspects 1-10 and 15-24, wherein the compound has the structure:

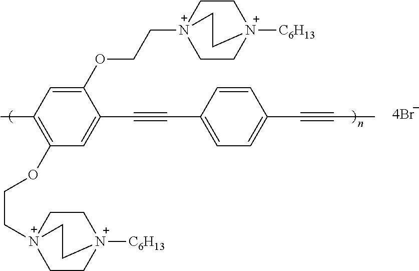

Aspect 33 provides the method of any one of Aspects 1-10 and 15-24, wherein the compound has the structure:

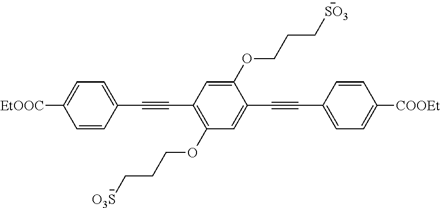

Aspect 34 provides the method of any one of Aspects 1-10 and 15-24, wherein the compound has the structure:

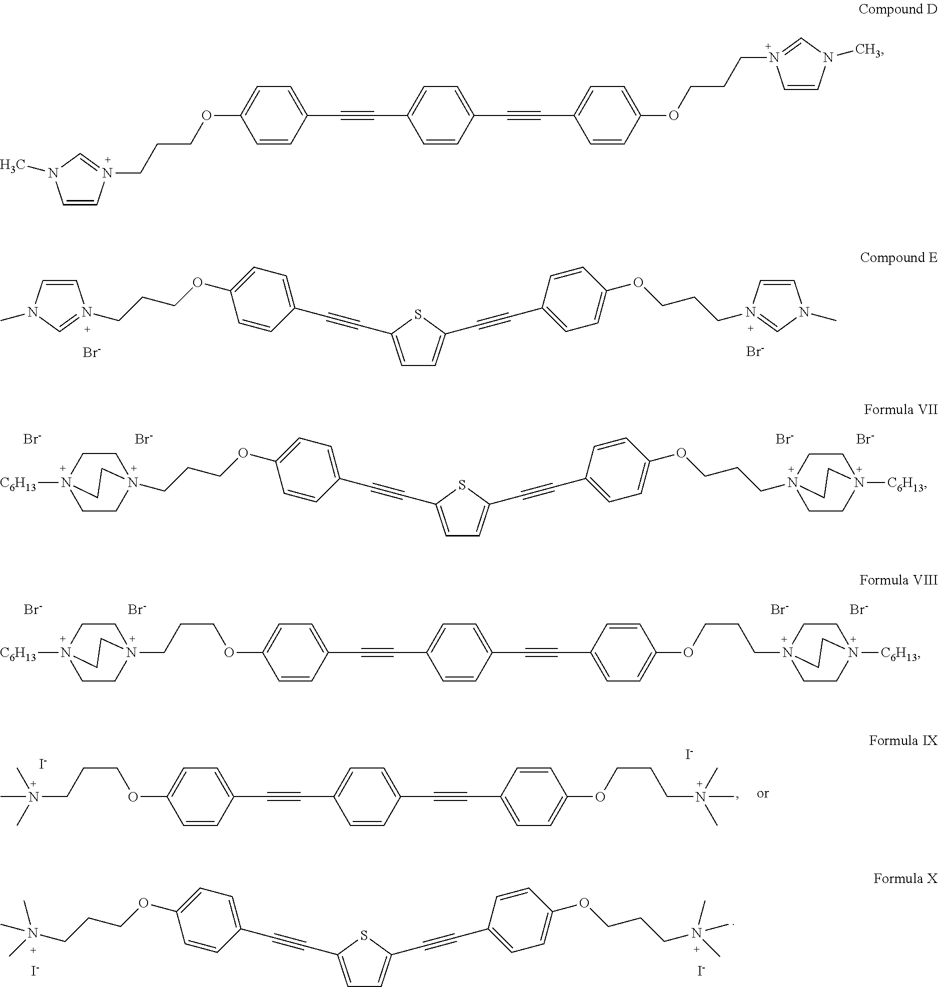

Aspect 35 provides the method of any one of Aspects 1-10 and 15-24, wherein the compound has a structure selected from the group consisting of:

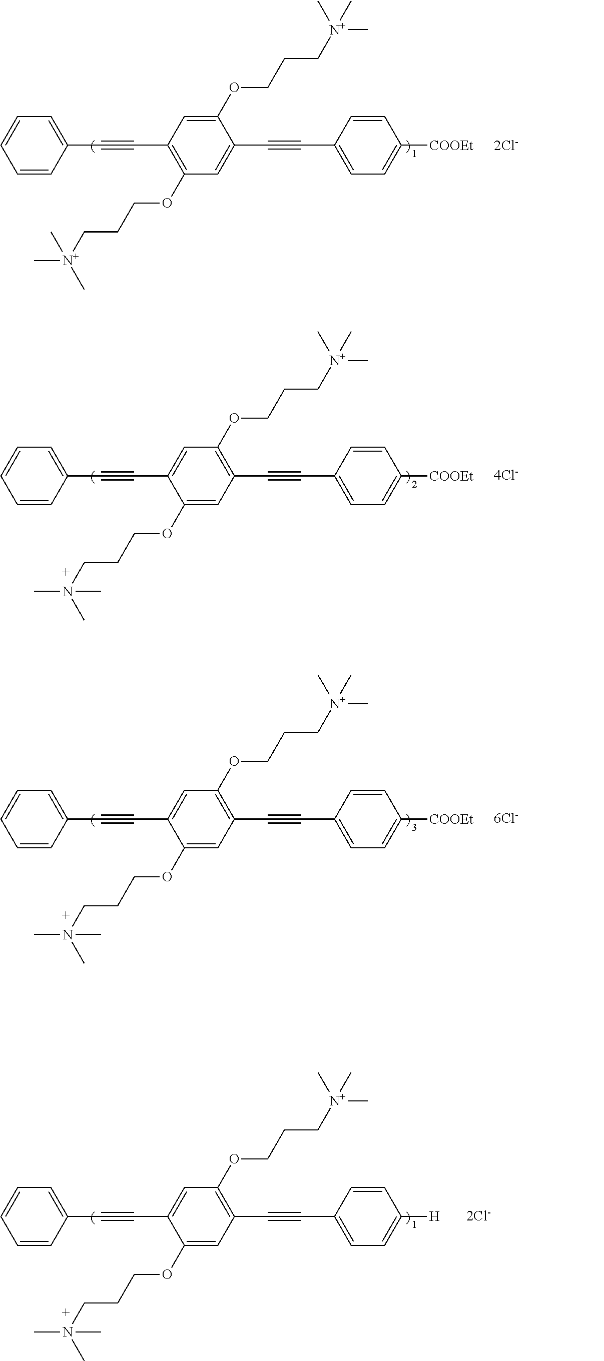

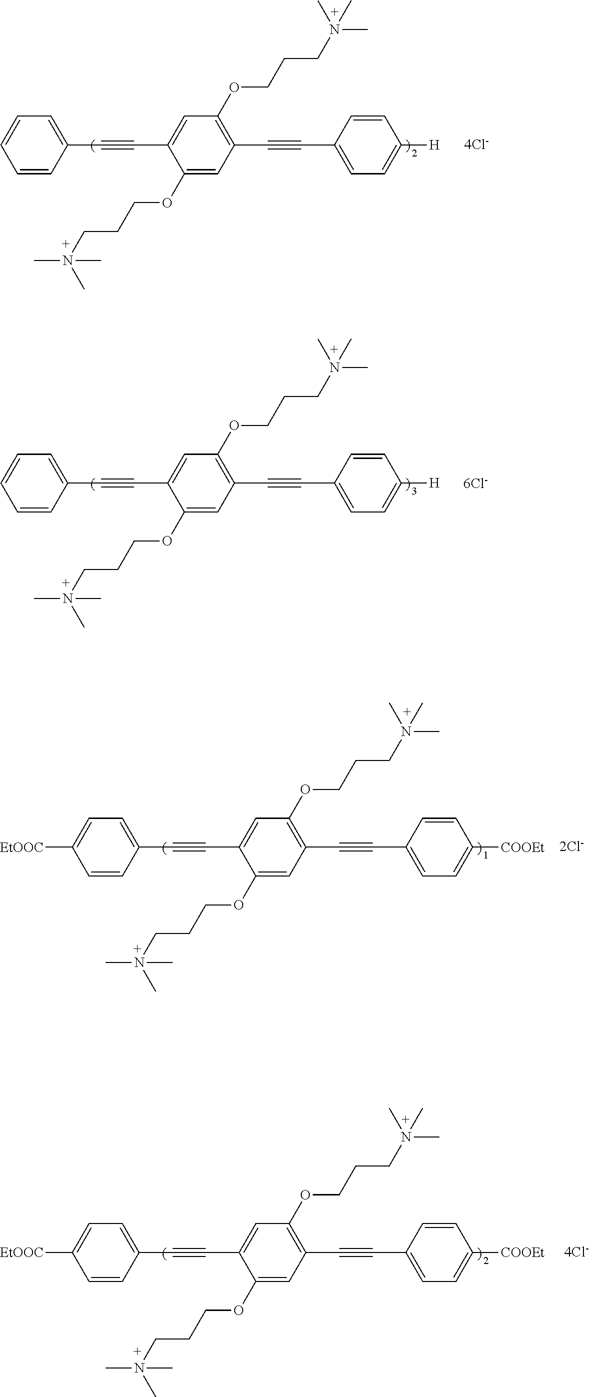

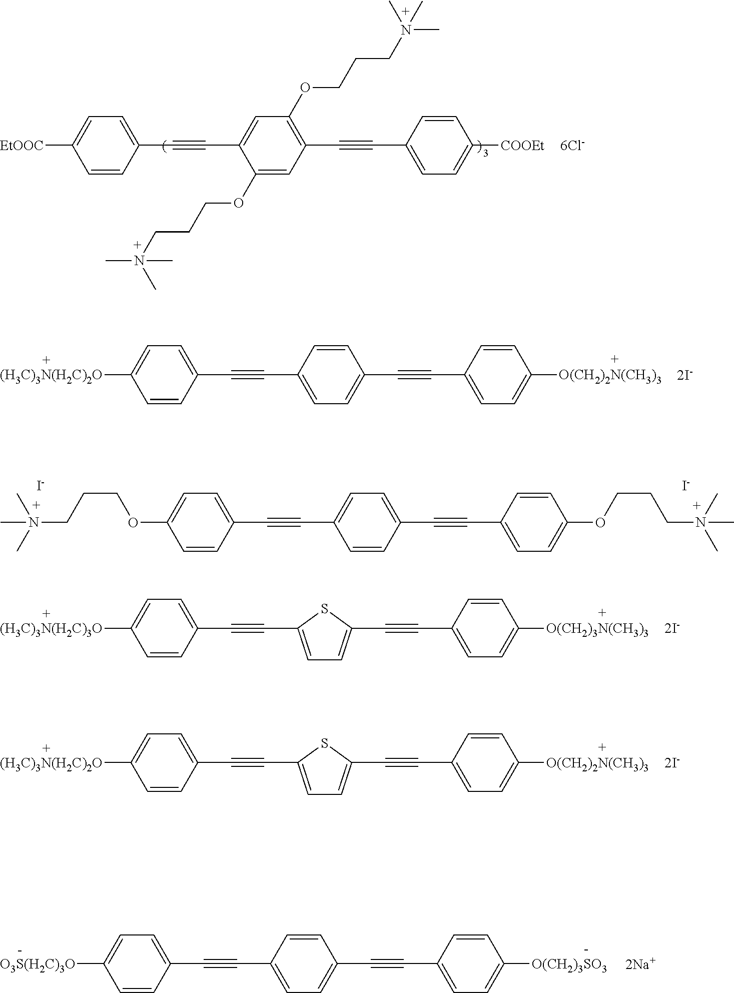

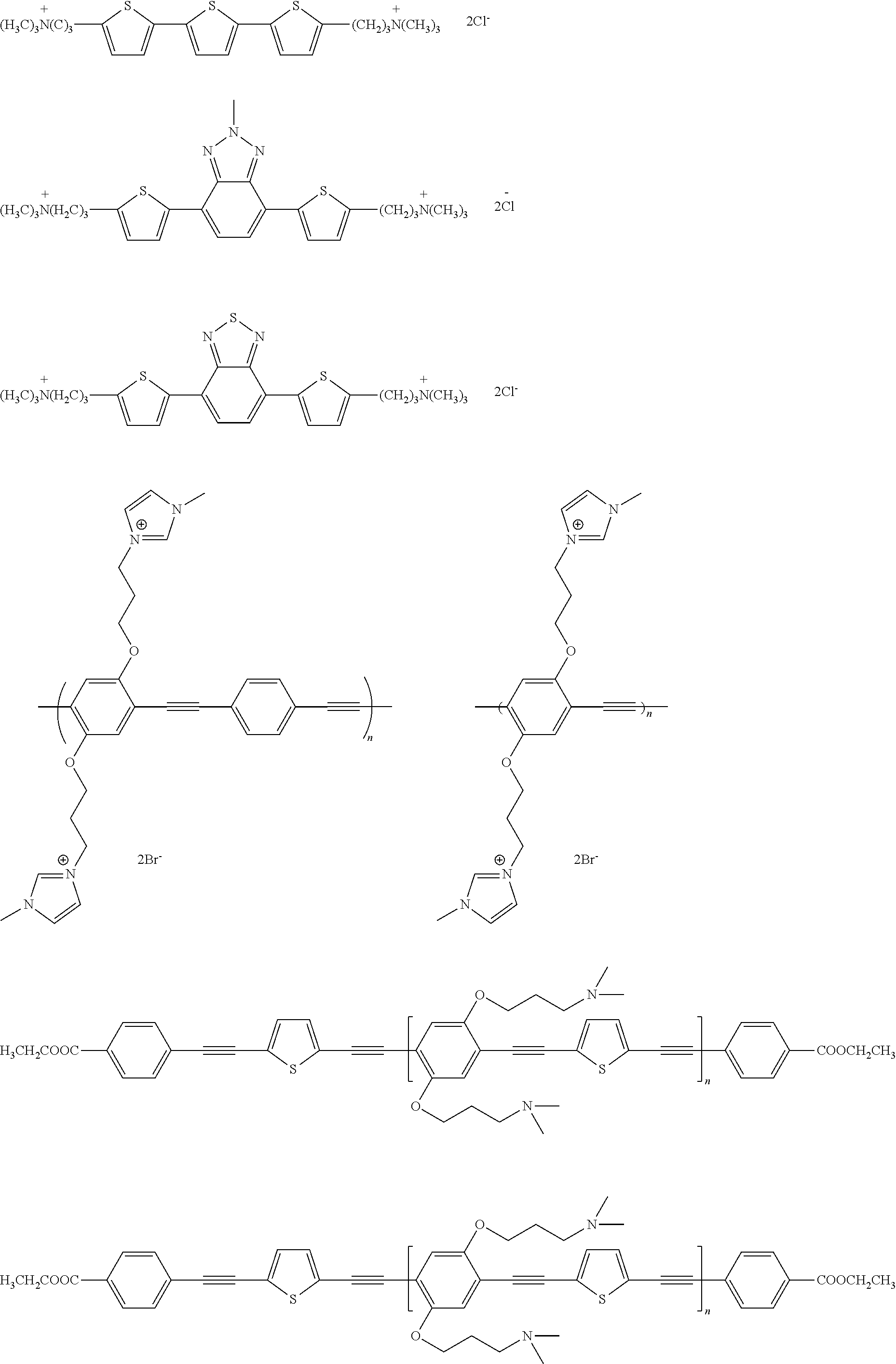

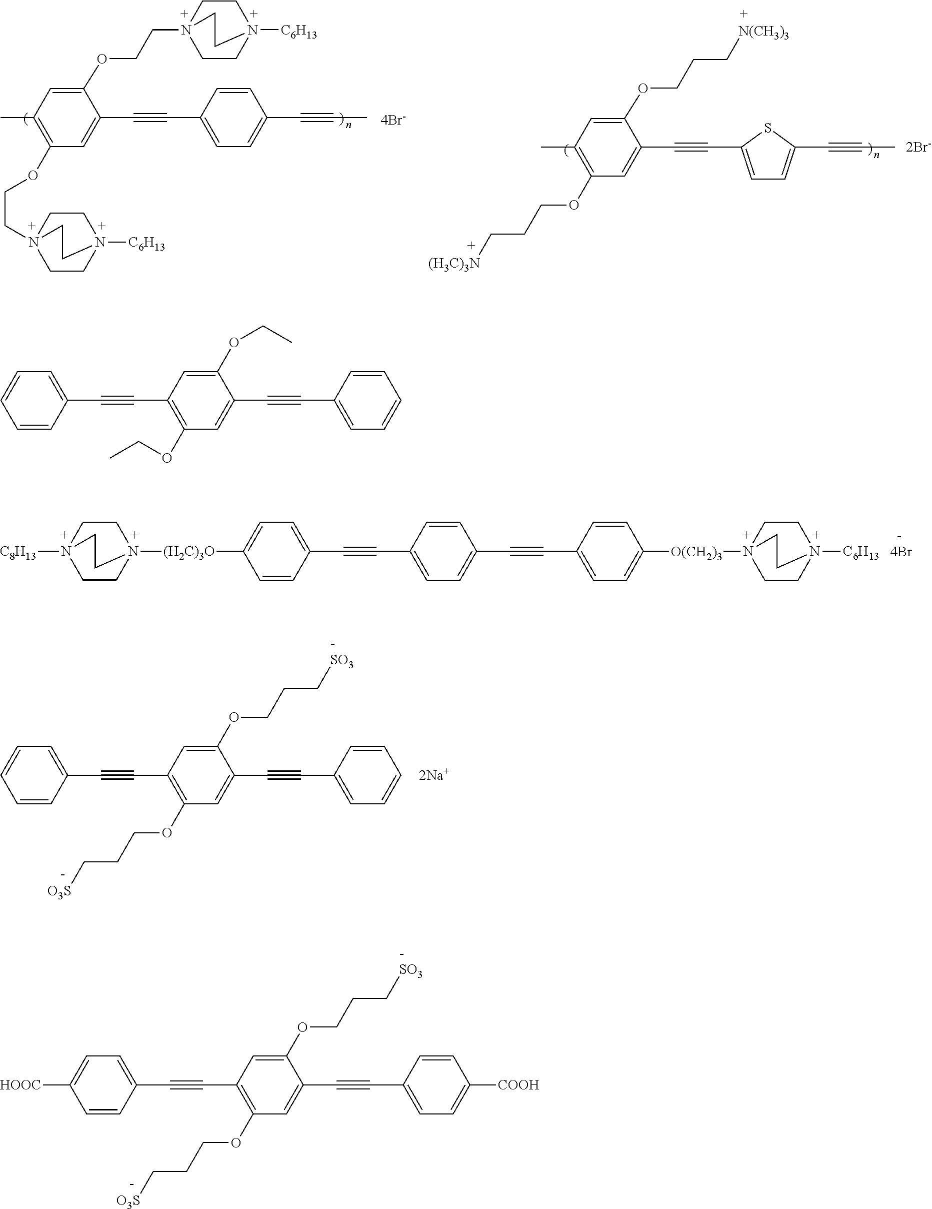

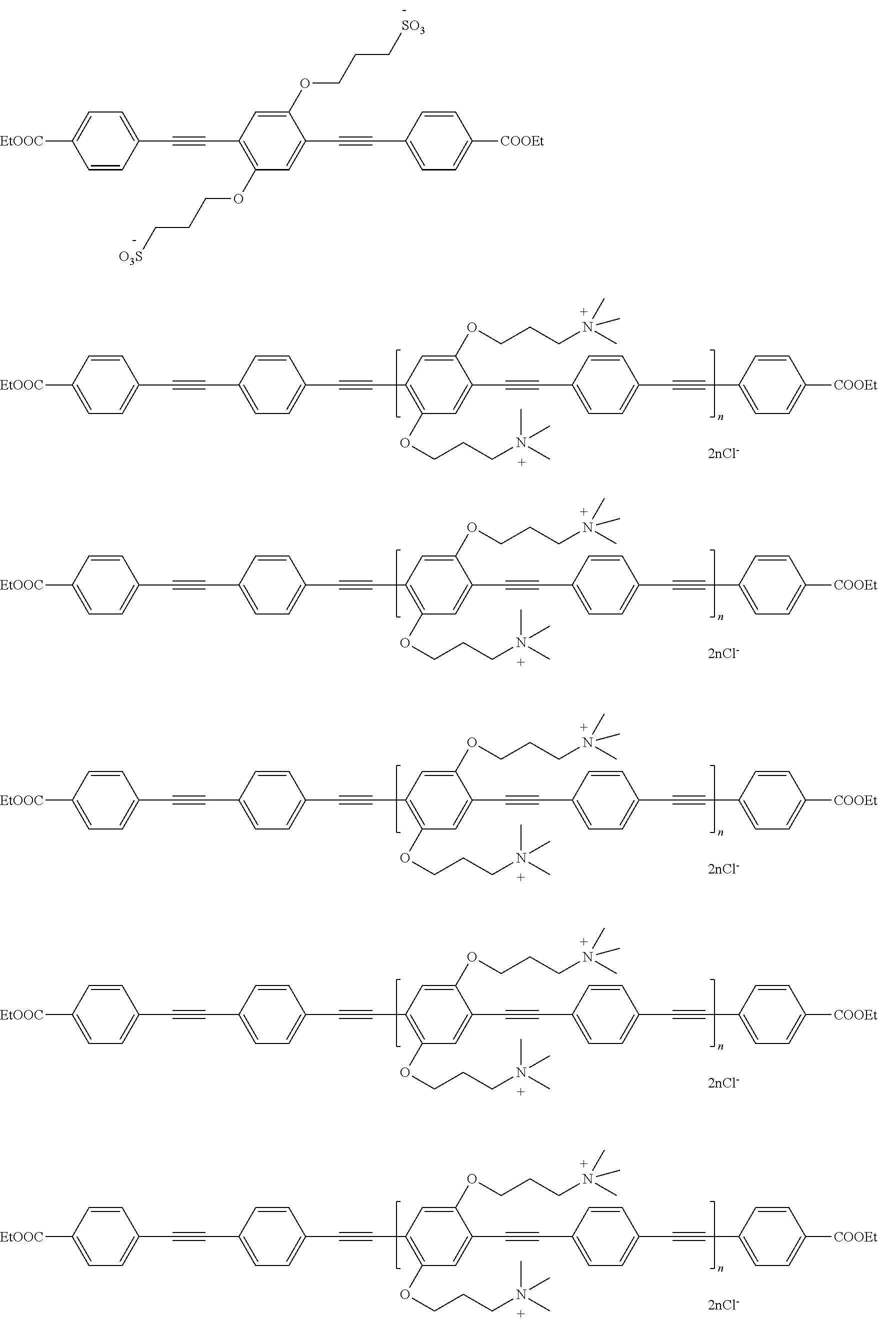

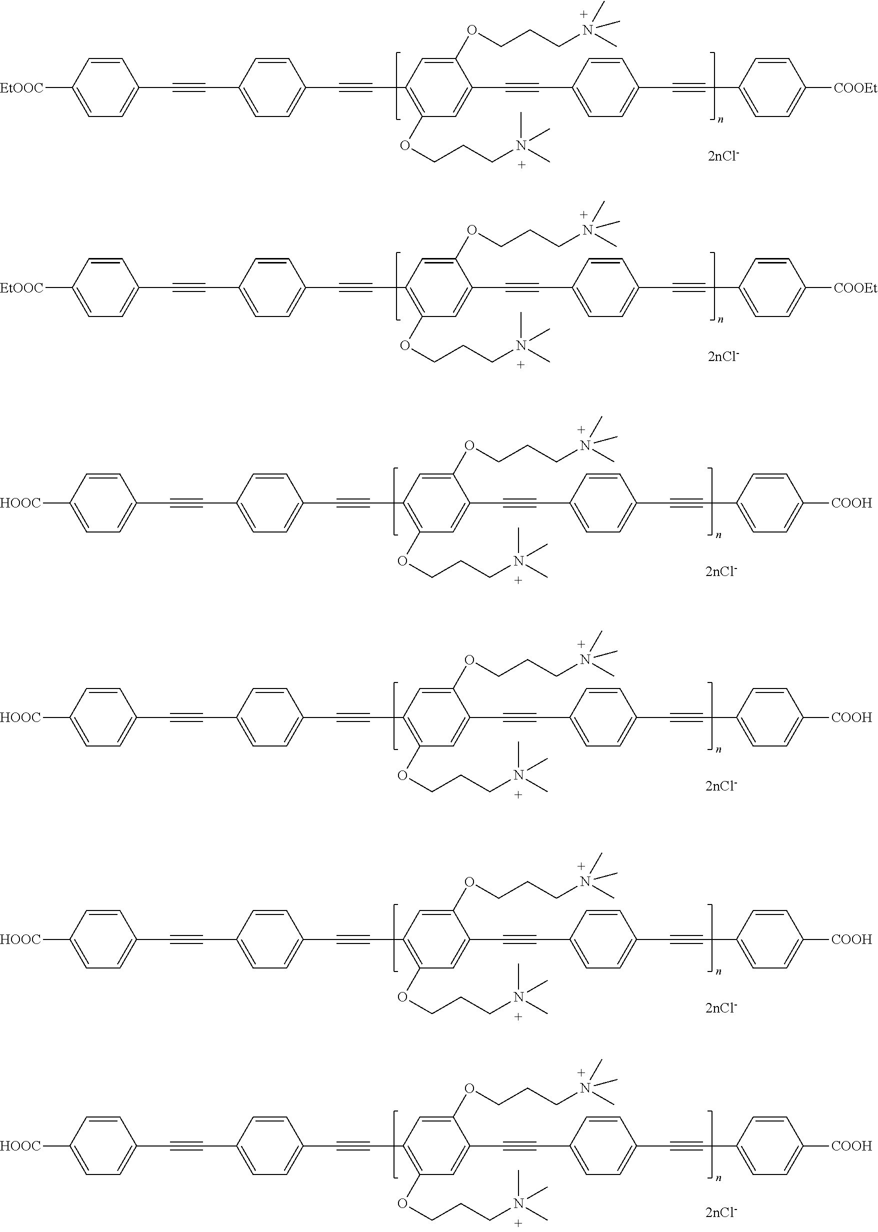

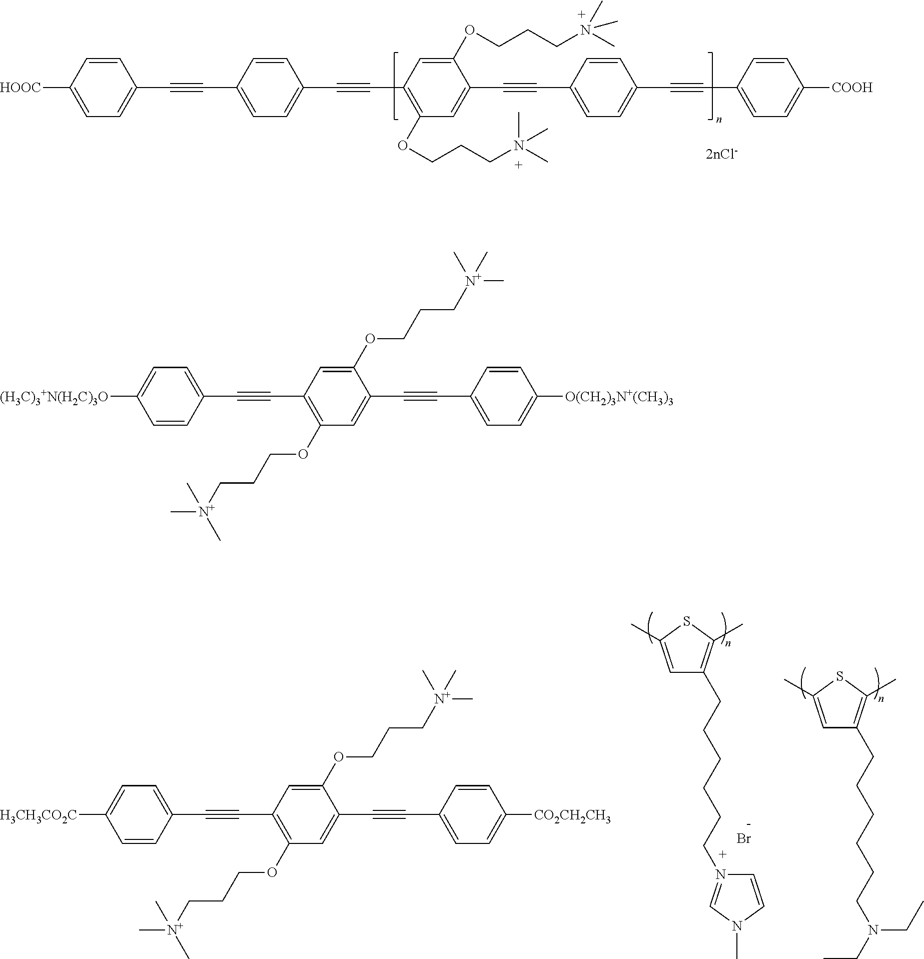

Aspect 36 provides a method of inhibiting an enveloped ssRNA virus, comprising contacting the virus with a composition comprising a compound having the structure of Formula Ib or Ic in the presence of light:

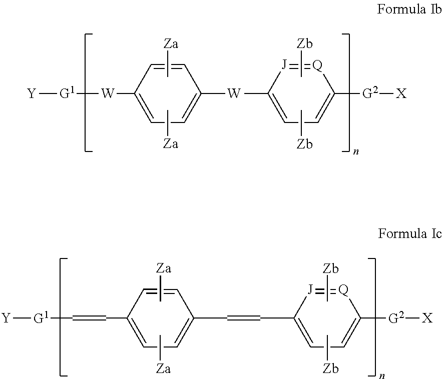

wherein

each of X and Y is independently H, COOR, O—(CH₂)ₘ-T, NH₂, or COR;

each of Za and Zb is independently H, O—(CH₂)ₘ-T, O—C₂H₄—(OCH₂)ₘ—R;

each of G¹ and G² is independently a bond, C₂C₆H₄. C₆H₄, C₂C₄S, or C₄S;

J and Q are each C or CH so as to provide a benzene ring, or J and Q are together S so as to provide a thiophene ring;

n is 1 to 200;

p is 1 to 10,000;

m is 0 to 10;

each of R is independently methyl, ethyl, n-propyl, isopropyl, phenyl, t-butyl, isobutyl, n-butyl, pentyl, hexyl, heptyl, octyl, cyclopropyl, cyclobutyl, cyclopentyl, cyclyhexyl, methoxymethyl, or trifluoromethyl;

each of T is independently H, SO₃⁻, COO⁻, COOR, DABCO, N-alkyl DABCO, imidazolyl, N-alkyl imidazolyl, NR₂, NHR₂⁺, or NR₃⁺;

each of W, if present, can independently be a bond, —C≡C—, or —C═C—; and

at least one occurrence of Y, X, Za, and Zb is independently O—(CH₂)ₘ-T.

Aspect 37 provides the method of Aspect 36, wherein the compound has the structure:

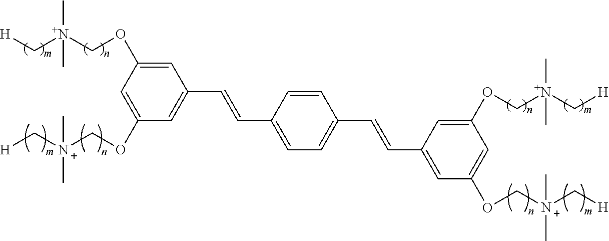

wherein m and n are each independently 1 to 10.

Aspect 38 provides a method of inactivating SARS-CoV-2 virus, the method comprising:

contacting SARS-CoV-2 virus with a conjugated aromatic compound effective to inactivate the virus.

Aspect 39 provides the method of Aspect 38, further comprising exposing the conjugated aromatic compound to light-irradiation while the conjugated oligomer or polymer is in contact with the SARS-CoV-2 virus.

Aspect 40 provides the method of any one of Aspects 38-39, wherein a coating, paint, wipe, spray, mask, clothing, personal protective equipment, warfare fighter, or combination thereof, comprises the conjugated aromatic compound.

Aspect 41 provides the method of any one of Aspects 38-40, wherein the conjugated aromatic compound comprises the structure:

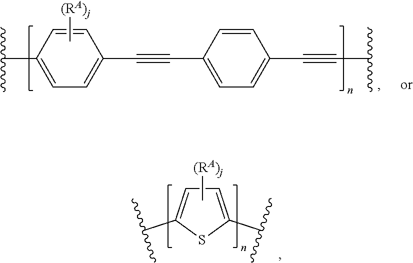

or wherein the conjugated aromatic compound has the structure:

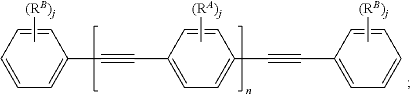

wherein

n is 1 to 10,000,

at each occurrence, j is independently 1 or 2,

R^{A} is —H or R^{C},

at each occurrence, R^{B} is independently —H or R^{C},

R^{C} is —X—R¹—R²,

X is a bond, —O—, —NH—, or —S—,

R¹ is substituted or unsubstituted (C₁-C₂₀)alkylene,

R² is -(1,4-substituted 1,4-diazabicyclo[2.2.2]octane-1,4-diium)-R³, 3-R³-substituted imidazolium, pyridinium, —SO₃⁻, —CO₂H, —CO₂⁻, —N⁺(R³)₃, —N⁺(R³)₂H, or —N(R³)₂,

R³ is —H or substituted or unsubstituted (C₁-C₁₀)alkane, and

at least one R^{A} or R^{B} in the compound is R^{C}.

Aspect 42 provides the method of Aspect 41, wherein j is 1.

Aspect 43 provides the method of any one of Aspects 41-42, wherein R^{A} is R^{C}.

Aspect 44 provides the method of any one of Aspects 41-43, wherein R^{B} is R^{C}.

Aspect 45 provides the method of any one of Aspects 41-44, wherein X is a bond.

Aspect 46 provides the method of any one of Aspects 41-44, wherein X is —O—.

Aspect 47 provides the method of any one of Aspects 41-46, wherein R¹ is (C₂-C₇)alkylene.

Aspect 48 provides the method of any one of Aspects 41-47, wherein R² is SO₃.

Aspect 49 provides the method of any one of Aspects 41-47, wherein R² is -(1,4-substituted 1,4-diazabicyclo[2.2.2]octane-1,4-diium)-C₆H₁₃.

Aspect 50 provides the method of any one of Aspects 41-47, wherein R² is 3-methylimidazolium.

Aspect 51 provides the method of any one of Aspects 41-44, wherein R^{C} is —O—(CH₂)₃—SO₃.

Aspect 52 provides the method of any one of Aspects 41-44, wherein R^{C} is —O—(CH₂)₃-3-methylimidazolium.

Aspect 53 provides the method of any one of Aspects 41-44, wherein R^{C} is —(CH₂)₇-3-methylimidazolium.

Aspect 54 provides the method of any one of Aspects 41-44, wherein R^{C} is —O—(CH₂)₂-(1,4-substituted 1,4-diazabicyclo[2.2.2]octane-1,4-diium)-C₆H₁₃.

Aspect 55 provides the method of any one of Aspects 41-54, wherein the conjugated aromatic compound comprises the structure:

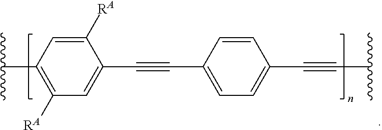

Aspect 56 provides the method of any one of Aspects 41-54, wherein the conjugated aromatic compound comprises the structure:

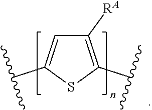

Aspect 57 provides the method of any one of Aspects 41-54, wherein the conjugated aromatic compound has the structure:

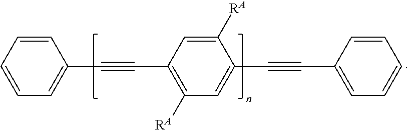

Aspect 58 provides the method of any one of Aspects 41-54, wherein the conjugated aromatic compound has the structure:

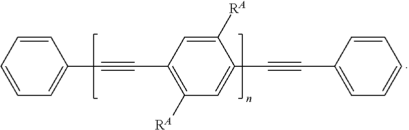

Aspect 59 provides the method of any one of Aspects 41-58, wherein the conjugated aromatic compound comprises one or more charge-balancing counterions.

Aspect 60 provides the method of any one of Aspects 38-41, wherein the conjugated aromatic compound has the structure:

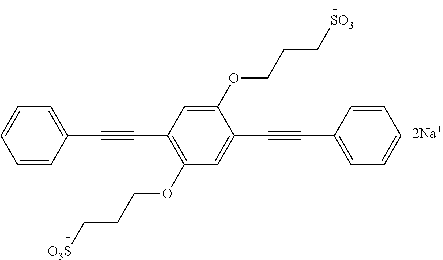

Aspect 61 provides the method of any one of Aspects 38-41, wherein the conjugated aromatic compound has the structure:

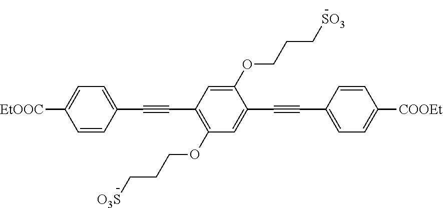

Aspect 62 provides the method of any one of Aspects 38-41, wherein the conjugated aromatic compound has the structure:

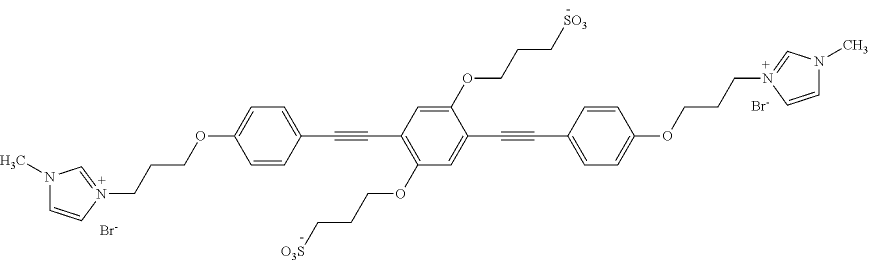

Aspect 63 provides the method of any one of Aspects 38-41, wherein the conjugated aromatic compound comprises the structure:

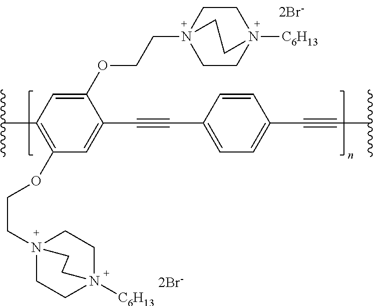

Aspect 64 provides the method of any one of Aspects 38-41, wherein the conjugated aromatic compound comprises the structure:

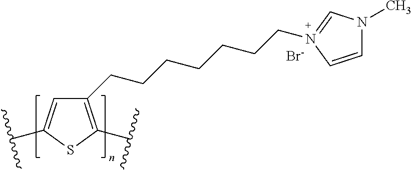

Aspect 65 provides the method of any one of Aspects 38-41, wherein the conjugated aromatic compound has the structure:

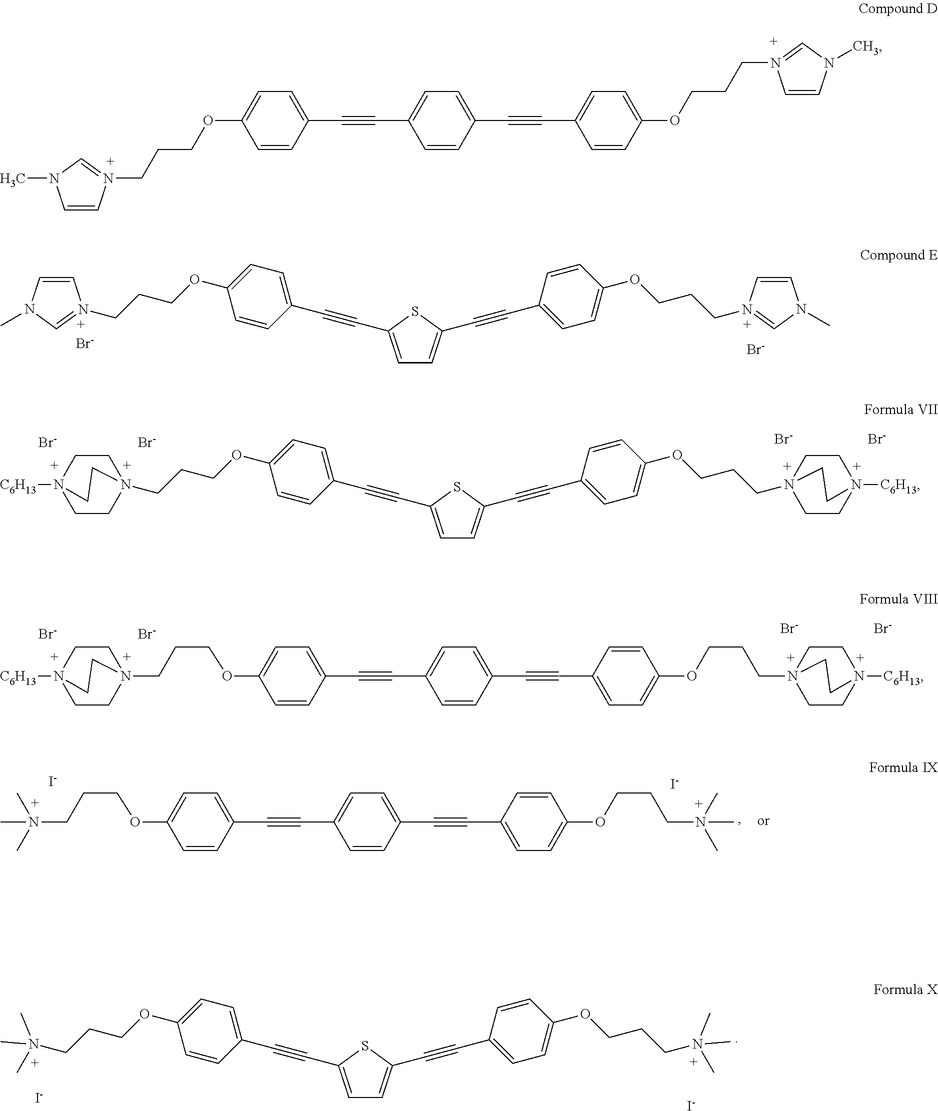

Aspect 66 provides the method of any one or any combination of Aspects 1-64 optionally configured such that all elements or options recited are available to use or select from.

What is claimed is:

1. A method of inhibiting an enveloped ssRNA virus, comprising contacting the virus with a composition comprising a compound having the structure of Formula I, Ib, Ic, or II, 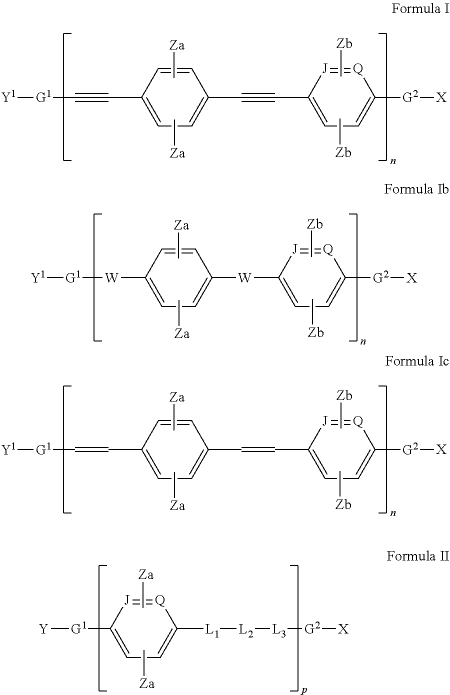
wherein
each of X and Y is independently H, COOR, O—(CH₂)ₘ-T, NH₂, or COR;
each of Za and Zb is independently H, O—(CH₂)ₘ-T, O—C₂H₄(OCH₂)ₘ—R;
each of G¹ and G² is independently a bond, C₂C₆H₄, C₆H₄, C₂C₄S, or C₄S;
J and Q are each C or CH so as to provide a benzene ring, or J and Q are together S so as to provide a thiophene ring;
n is 1 to 200;
p is 1 to 10,000;
m is 0 to 10;
each of R is independently methyl, ethyl, n-propyl, isopropyl, phenyl, t-butyl, isobutyl, n-butyl, pentyl, hexyl, heptyl, octyl, cyclopropyl, cyclobutyl, cyclopentyl, cyclyhexyl, methoxymethyl, or trifluoromethyl;
each of T is independently H, SO₃⁻, COO⁻, COOR, DABCO, N-alkyl DABCO, imidazolyl, N-alkyl imidazolyl, NR₂, NHR₂⁺, or NR₃⁺;
L1 is independently a bond or —C≡C—;
L2 is independently a bond, a substituted or unsubstituted phenylene, thiophenylene, azulenylene, heptalenylene, biphenylene, indacenylene, fluorenylene, phenanthrenylene, triphenylenylene, pyrenylene, naphthacenylene, chrysenylene, biphenylenylene, anthracenylene, and naphthylene;
L3 is independently a bond or —C—═C—;
each of W, if present, can independently be a bond, —C≡C—, or —C═C—; and
at least one occurrence of Y, X, Za, and Zb is independently O—(CH₂)ₘ-T.

2. The method of claim 1, wherein the enveloped ssRNA virus is SARS-CoV-2.

3. The method of claim 1, wherein the contacting is in the presence of visible light and/or UV light.

4. The method of claim 1, wherein the compound has a molecular weight of less than 2000 Da.

5. The method of claim 1, wherein the compound has the structure of formula I and n=1.

6. The method of claim 1, wherein the compound has the structure of Formula II, and wherein:
each of X and Y is independently H or COOR;
each of Za is O—(CH₂)ₘ-T;
G¹ is C₆H₄;
G² is a bond;
J and Q are each CH so as to provide a benzene ring;
n is 1;
p is 1 to 10,000;
m is 2 to 3;
each of R is methyl or ethyl;
each of T is SO₃⁻, N-hexyl DABCO, or N-methyl imidazolyl;
L1 is —C≡C—;
L2 is unsubstituted phenylene; and
L3 is —C≡C—.

7. The method of claim 1, wherein the method is a method of reducing SARS-CoV-2 viability on a substrate, the method further comprising treating a substrate with the composition and exposing the substrate to SARS-CoV-2 in the presence of light.

8. The method of claim 1, wherein the substrate is a wipe, a tissue, a bandage, a medical device, a surgical instrument, a sponge, a textile, a diaper, a counter-top, a food preparation surface, a wound dressing, a dressing for surgical incisions, a keyboard surface, a packing for wounds, a packing for surgical incisions, a nasal packing, a feminine care product, a fabric, a work surface, a medical device, packaging materials, personal protective equipment, a fibrous support, a wipe, a water filter, an air filter, a mask, or a combination thereof.

9. The method of claim 1, wherein the compound has the structure of Formula V 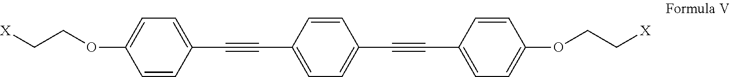
wherein X is n-hexyl DABCO, n-methyl imidazolium, or trimethylammonium.

10. The method of claim 1, wherein the compound has the structure:

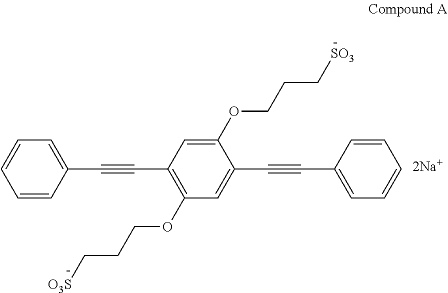

11. The method of claim 1, wherein the compound has the structure:

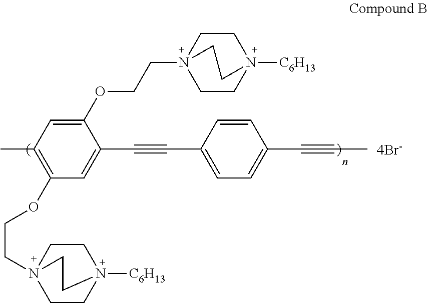

12. The method of claim 1, wherein the compound has the structure:

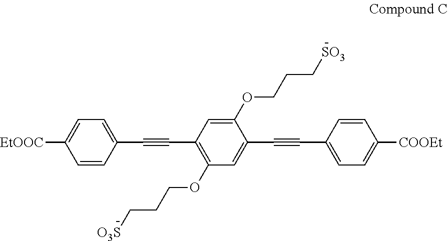

13. The method of claim 1, wherein the compound has the structure:

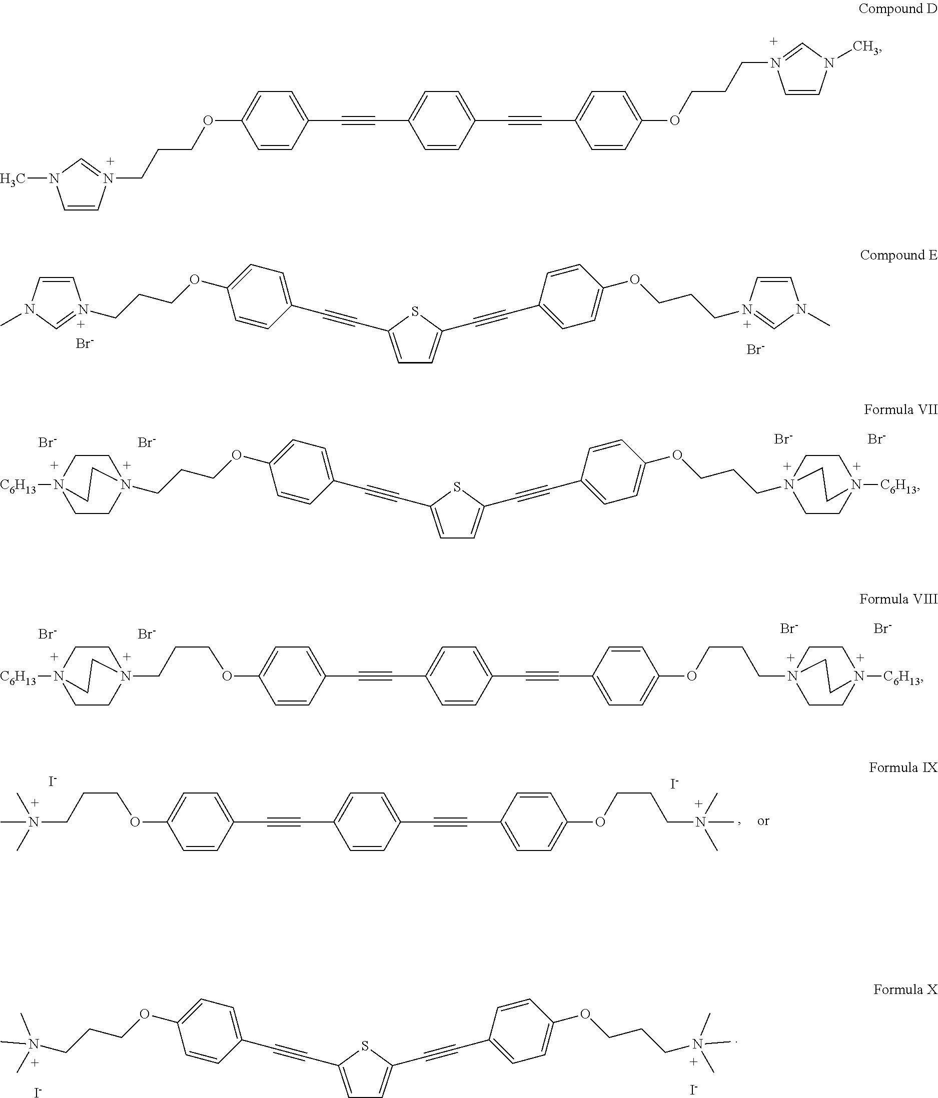

14. The method of claim 1, wherein the compound has the structure: 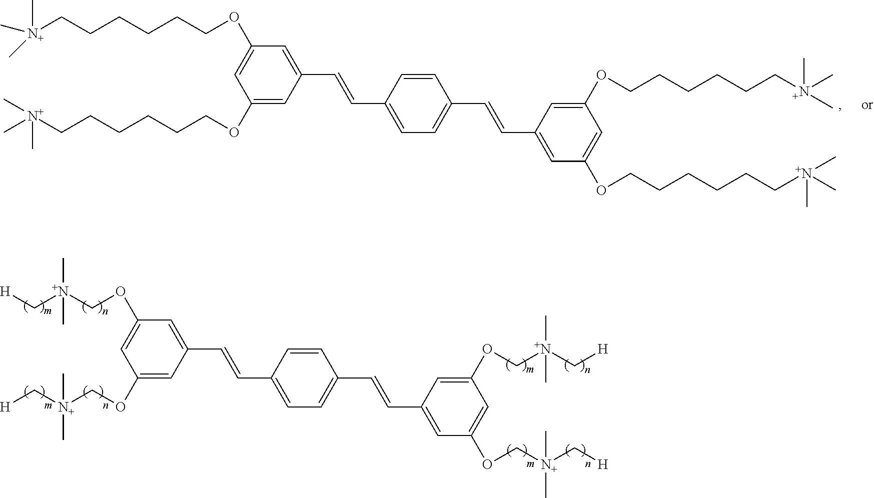
wherein m and n are each independently 1 to 10.

15. The method of claim 1, wherein the compound has a structure selected from the group consisting of:

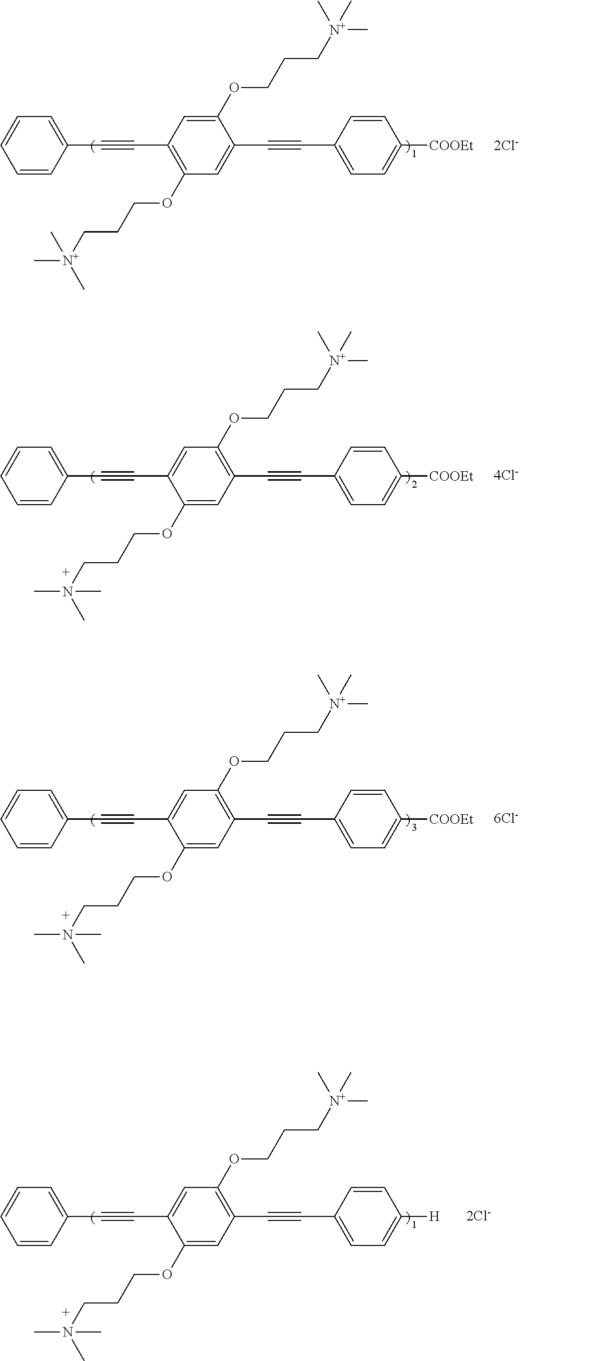

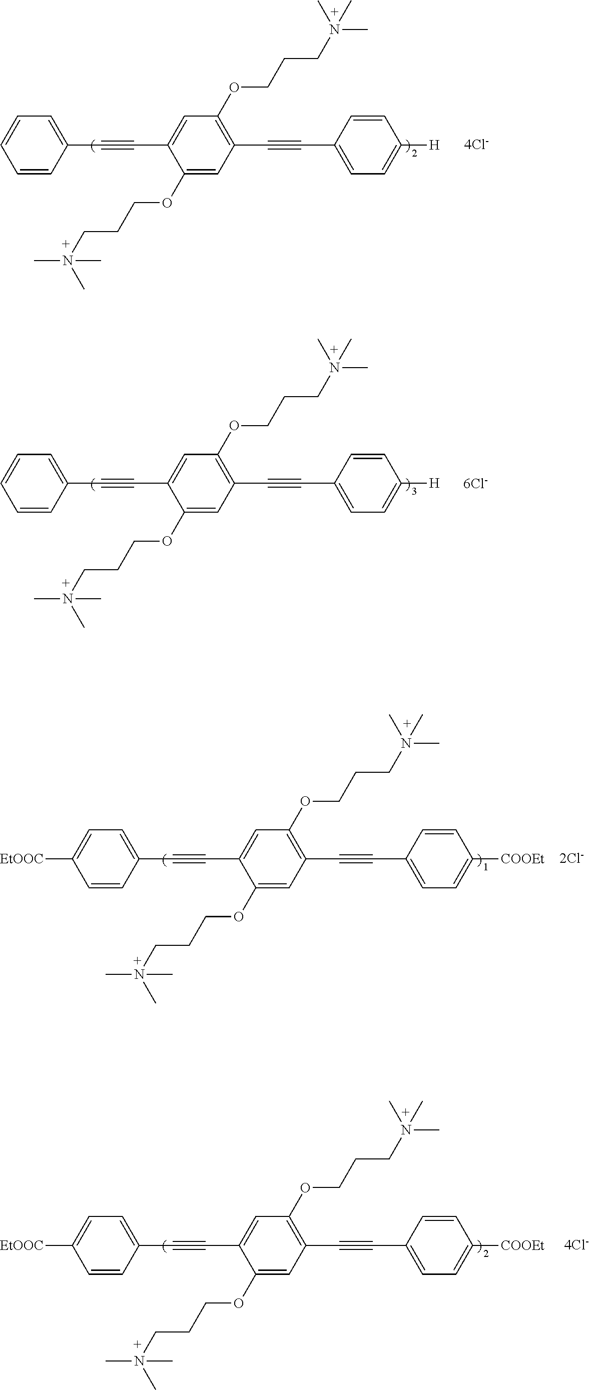

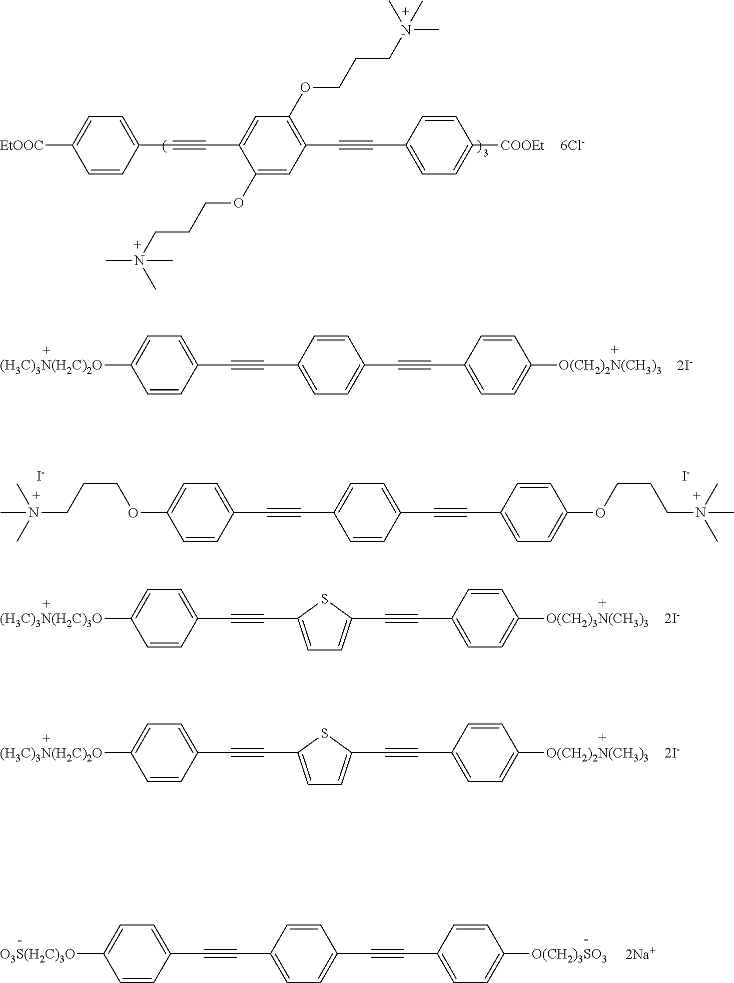

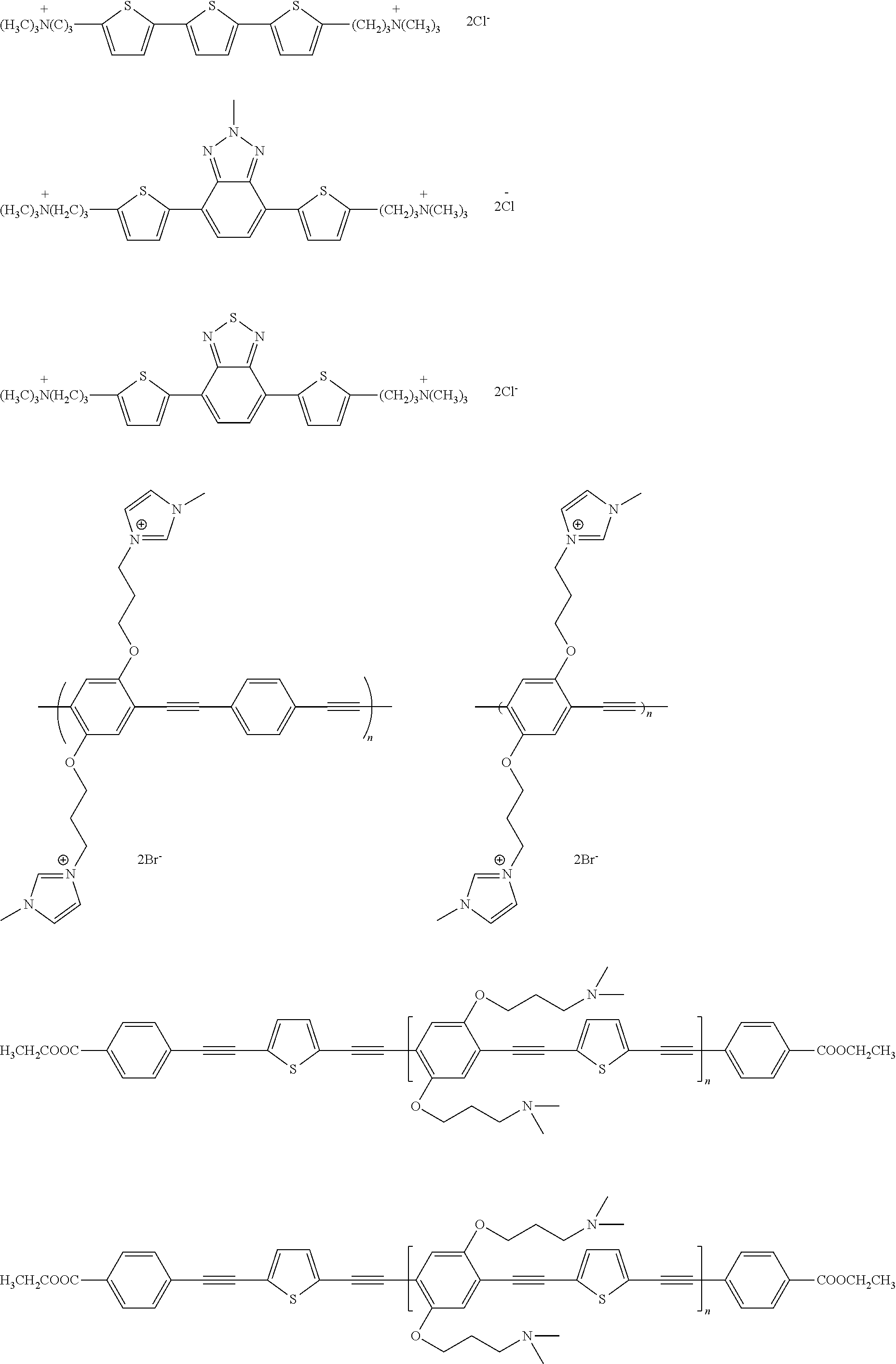

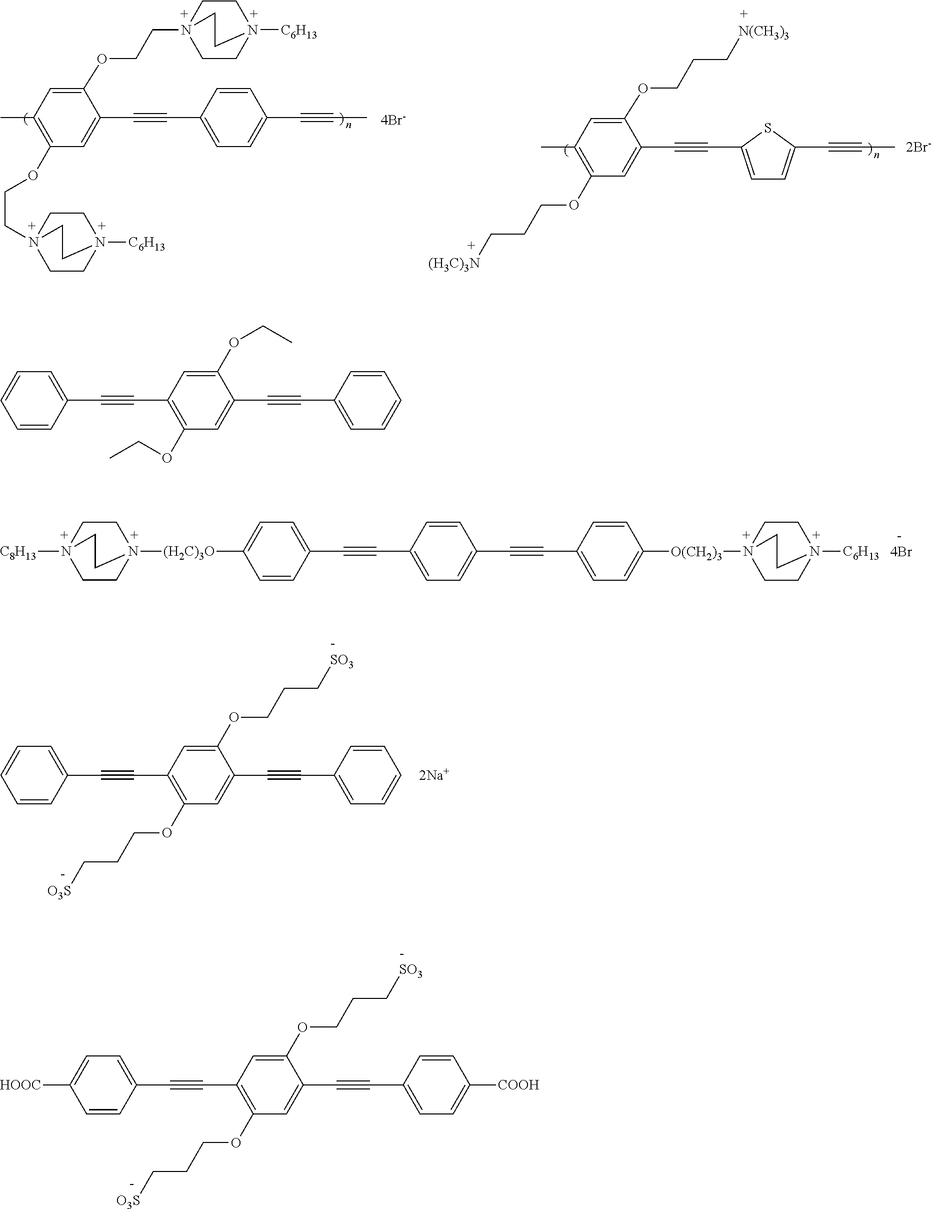

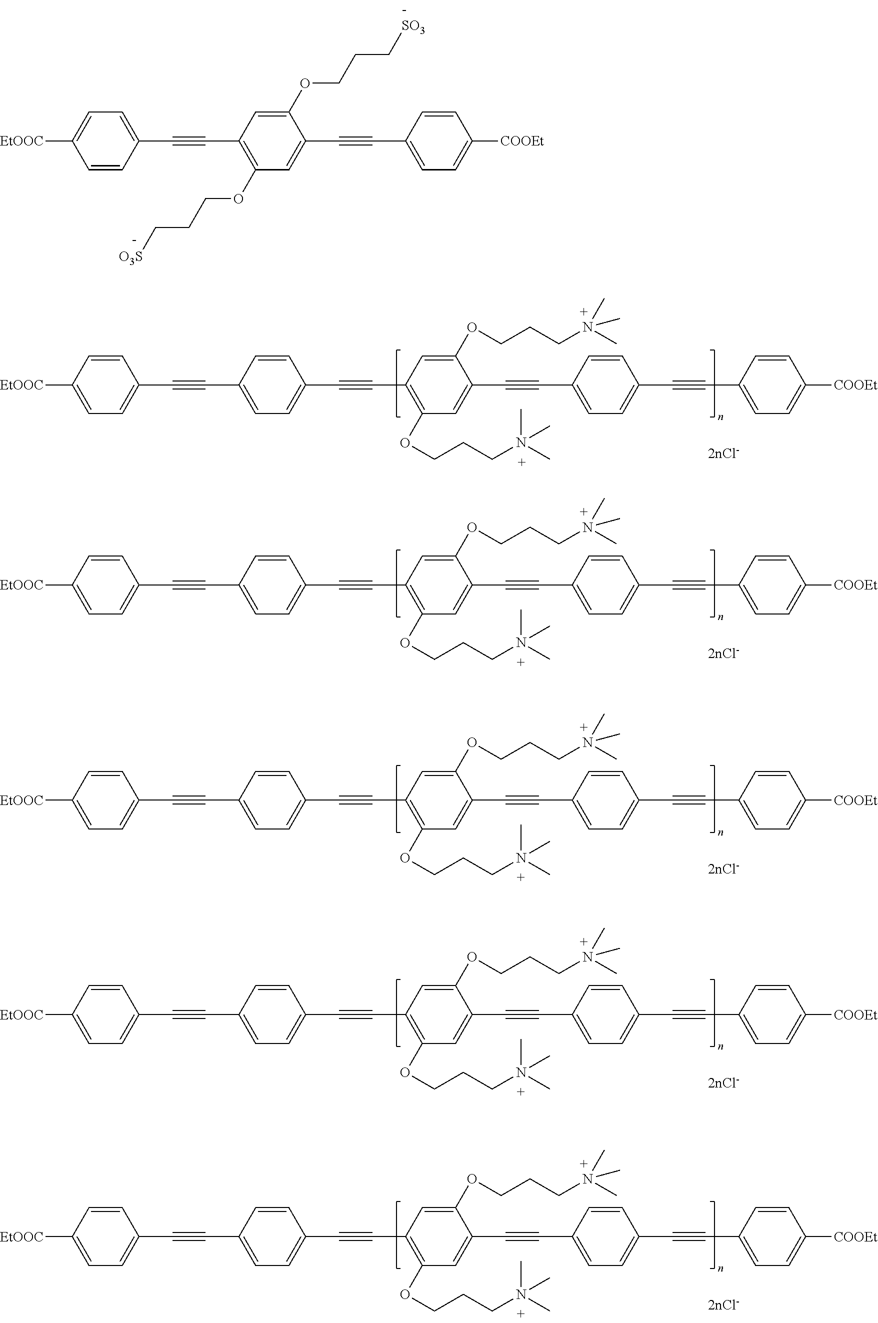

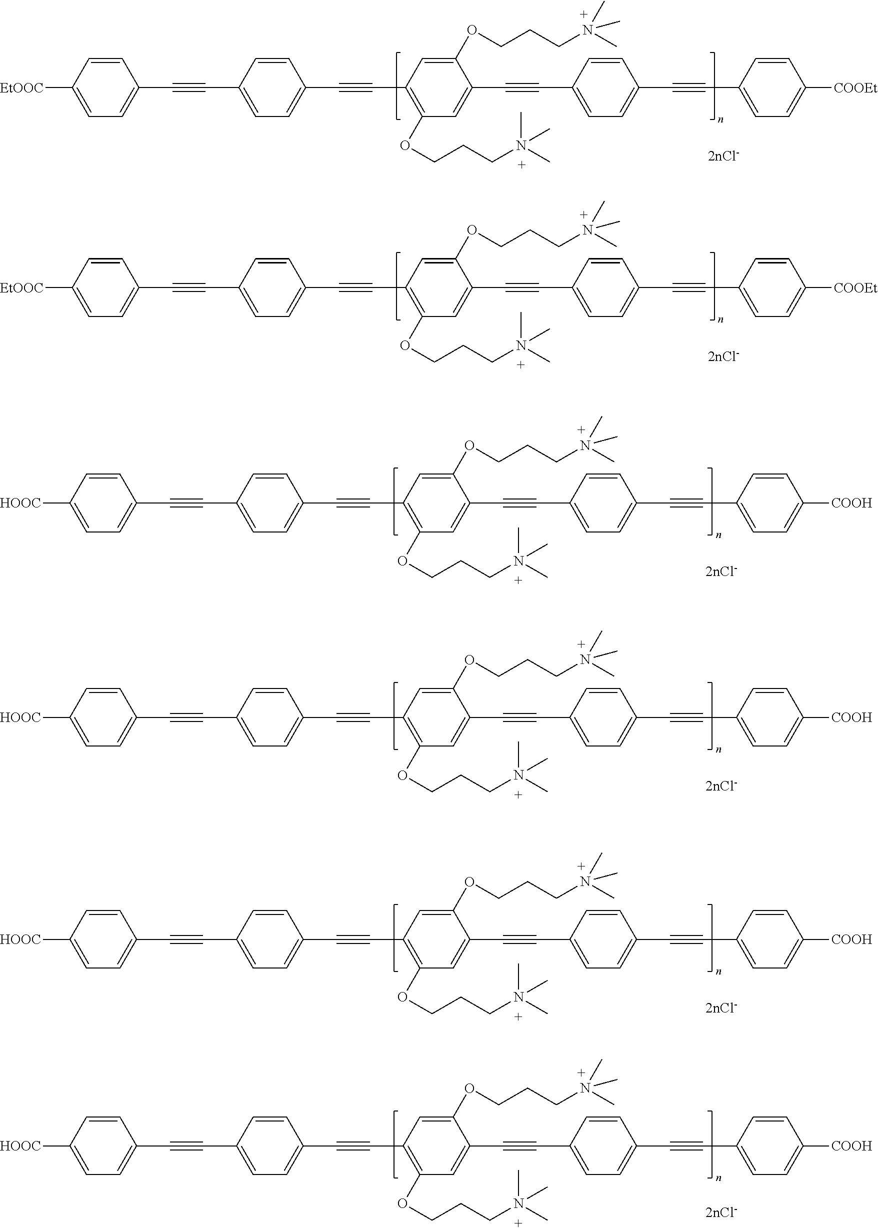

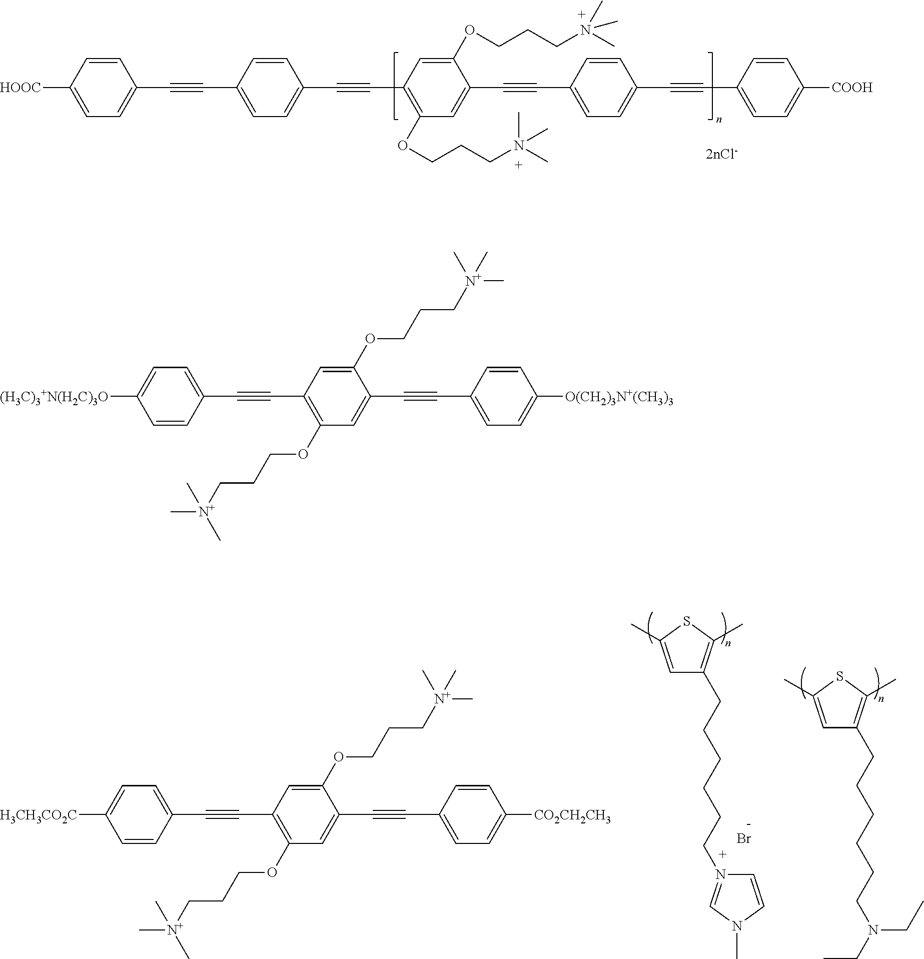

16. A method of inhibiting SARS-CoV-2, comprising:
treating a substrate surface with a composition comprising a compound having the structure of Formula I, Ib, Ic, or II, 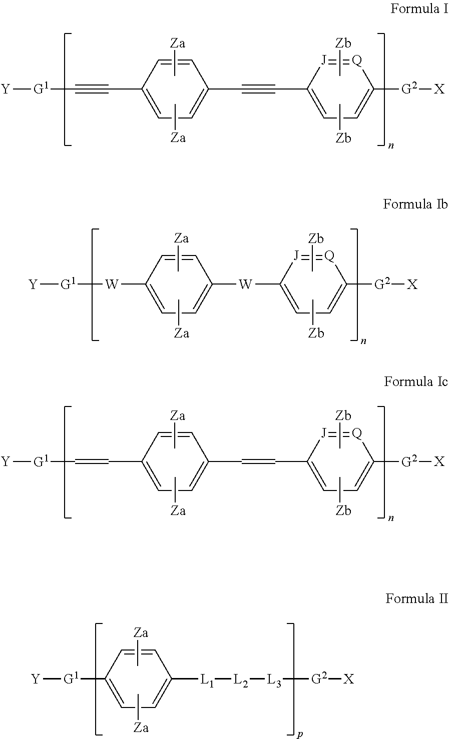
wherein
each of X and Y is independently H, COOR, O—(CH₂)ₘ-T, NH₂, or COR;
each of Za and Zb is independently H, O—(CH₂)ₘ-T, O—C₂H₄—(OCH₂)ₘ—R;
each of G¹ and G² is independently a bond, C₂C₆H₄, C₆H₄, C₂C₄S, or C₄S;
J and Q are each C or CH so as to provide a benzene ring, or J and Q are together S so as to provide a thiophene ring;
n is 1 to 200;
p is 1 to 10,000;
m is 0 to 10;
each of R is independently methyl, ethyl, n-propyl, isopropyl, phenyl, t-butyl, isobutyl, n-butyl, pentyl, hexyl, heptyl, octyl, cyclopropyl, cyclobutyl, cyclopentyl, cyclyhexyl, methoxymethyl, or trifluoromethyl;
each of T is independently H, SO₃⁻, COO⁻, COOR, DABCO, N-alkyl DABCO, imidazolyl, N-alkyl imidazolyl, NR₂, NHR₂⁺, or NR₃⁺;
L1 is independently a bond or —C≡C—;
L2 is independently a bond, a substituted or unsubstituted phenylene, thiophenylene, azulenylene, heptalenylene, biphenylene, indacenylene, fluorenylene, phenanthrenylene, triphenylenylene, pyrenylene, naphthacenylene, chrysenylene, biphenylenylene, anthracenylene, and naphthylene;
L3 is independently a bond or —C≡C—;
each of W, if present, can independently be a bond, —C≡C—, or —C═C—; and
at least one occurrence of Y, X, Za, and Zb is independently O—(CH₂)ₘ-T.

17. The method of claim 16, wherein
the method is a method of inhibiting SARS-CoV-2 in circulated air, wherein the substrate surface comprises an air filter, or
the method is a method of inhibiting SARS-CoV-2 in water, wherein the substrate surface comprises a water filter.

18. A method of inactivating SARS-CoV-2 virus, the method comprising:
contacting SARS-CoV-2 virus with a conjugated aromatic compound effective to inactivate the virus.

19. The method of claim 18, wherein the conjugated aromatic compound comprises the structure: 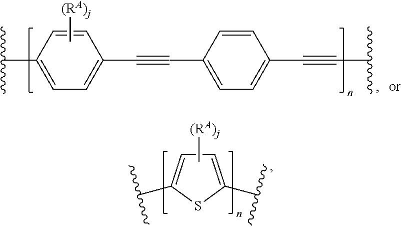
or wherein the conjugated aromatic compound has the structure: 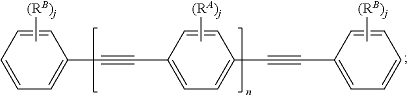
wherein
n is 1 to 10,000,
at each occurrence, j is independently 1 or 2,
R^{A} is —H or R^{C},
at each occurrence, R^{B} is independently —H or R^{C},
R^{C} is —X—R¹—R²,
X is a bond, —O—, —NH—, or —S—,
R¹ is substituted or unsubstituted (C₁-C₂₀)alkylene,
R² is -(1,4-substituted 1,4-diazabicyclo[2.2.2]octane-1,4-diium)-R³, 3-R³-substituted imidazolium, pyridinium, —SO₃⁻, —CO₂H, —CO₂⁻, —N⁺(R³)₃, —N⁺(R³)₂H, or —N(R³)₂,
R³ is —H or substituted or unsubstituted (C₁-C₁₀)alkane, and
at least one R^{A} or R^{B} in the compound is R^{C}.

20. The method of claim 18, wherein the conjugated aromatic compound comprises the structure: [chemical structure]
